(19) United States
(12) Patent Application Publication
(10) Pub. No.: US 2024/0209368 A1
(43) Pub. Date: Jun. 27, 2024

(54) EXTRACELLULAR VESICLE COMPOSITIONS

(71) Applicant: LONZA SALES AG, Basel (CH)

(72) Inventors: Conlin O'NEIL, Cambridge, MA (US); Raymond BOURDEAU, Cambridge, MA (US); Kimberly ELLIS, Cambridge, MA (US); Andrew WOOD, Cambridge, MA (US)

(21) Appl. No.: 18/552,778

(22) PCT Filed: Apr. 1, 2022
(86) PCT No.: PCT/US2022/023120

§ 371 Date: Sep. 27, 2023

Related U.S. Application Data
(60) Provisional application No. 63/169,751, filed on Apr. 1, 2021.

(51) Int. Cl. C12N 15/113; A61K 47/02; A61K 47/26; A61K 47/64; A61K 47/69

(52) U.S. Cl. CPC C12N 15/113; A61K 47/02; A61K 47/26; A61K 47/64; A61K 47/6901; C12N 2310/11

(57) ABSTRACT

The present disclosure relates to compositions comprising extracellular vesicles (e.g., exosomes) that can comprise an antisense oligonucleotide (ASO), wherein the composition has an osmolarity that is lower than 450 mOsm/kg. Also provided herein are methods for producing the extracellular vesicles and methods for using the extracellular vesicles to treat and/or prevent a range of medical disorders.

### CROSS-REFERENCE TO RELATED APPLICATIONS

This application claims priority benefit of U.S. Provisional Application No. 63/169,751, filed Apr. 1, 2021, which is incorporated by reference herein in its entirety.

### REFERENCE TO SEQUENCE LISTING SUBMITTED ELECTRONICALLY VIA EFS-WEB

The content of the electronically submitted sequence listing (Name: 4000_127PC01_Seqlisting_ST25.txt, Size: 66,571 bytes; and Date of Creation: Mar. 31, 2022) submitted in this application is incorporated herein by reference in its entirety.

### FIELD OF DISCLOSURE

The present disclosure relates to compositions for the storage and administration of extracellular vesicles (EVs), e.g., exosomes, that can comprise one or more exogenous biologically active moieties, and methods of preparing and using such compositions.

### BACKGROUND

EVs, e.g., exosomes, are important mediators of intercellular communication. They are also important biomarkers in the diagnosis and prognosis of many diseases, including cancer. As drug delivery vehicles, EVs offer advantages over traditional drug delivery methods (e.g., peptide immunization, DNA vaccines) as a new treatment modality in many therapeutic areas. One area of research is the formulation of compositions which can stably comprise EVs during lengthy storage periods prior to patient administration, without compromising the efficacy of the EVs. Known formulations suffer from drawbacks. For example, certain formulations e.g., those containing TRIS buffer, do not prevent the pH from fluctuating at various temperatures (i.e., when the formulation is frozen or thawed). Even small variations in pH can induce aggregation of EVs, thereby reducing or preventing their functionality. Further, known compositions include extraneous components such as exogenously added polypeptides, e.g., human serum albumin, or chelating agents.

Accordingly, there is a need for effective compositions for the storage and administration of EVs which overcome the drawbacks of known formulations, and can thus better enable the therapeutic use and other applications of EV-based technologies.

### BRIEF SUMMARY OF THE DISCLOSURE

Some aspects of the present disclosure are directed to a pharmaceutical composition comprising an extracellular vesicle comprising an antisense oligonucleotide (ASO), wherein the ASO comprises a contiguous nucleotide sequence of 10 to 30 nucleotides in length that is complementary to a nucleic acid sequence within a STAT6 transcript; wherein the composition has an osmolarity of lower than about 450 mOsm/kg.

In some aspects, the osmolarity of the composition is at least about 300 to about 450 mOsm/kg, at least about 310 to about 450 mOsm/kg, at least about 320 to about 450 mOsm/kg, at least about 330 to about 450 mOsm/kg, at least about 340 to about 450 mOsm/kg, at least about 350 to about 450 mOsm/kg, at least about 355 to about 450 mOsm/kg, at least about 360 to about 450 mOsm/kg, at least about 365 to about 450 mOsm/kg, at least about 365 to about 445 mOsm/kg, at least about 365 to about 440 mOsm/kg, at least about 365 to about 435 mOsm/kg, at least about 365 to about 430 mOsm/kg, at least about 365 to about 425 mOsm/kg, at least about 370 to about 420 mOsm/kg, at least about 375 to about 415 mOsm/kg, at least about 380 to about 410 mOsm/kg, at least about 385 to about 405 mOsm/kg, at least about 390 to about 400 mOsm/kg, at least about 395 to about 400 mOsm/kg, or at least about 390 to about 395 mOsm/kg. In some aspects, the osmolarity of the composition is at least about 365 to about 425 mOsm/kg. In some aspects, the osmolarity of the composition is about 365 mOsm/kg, about 370 mOsm/kg, about 375 mOsm/kg, about 380 mOsm/kg, about 385 mOsm/kg, about 390 mOsm/kg, about 395 mOsm/kg, about 400 mOsm/kg, about 405 mOsm/kg, about 410 mOsm/kg, about 415 mOsm/kg, about 420 mOsm/kg, or about 425 mOsm/kg. In some aspects, the osmolarity of the composition is about 395 mOsm/kg.

In some aspects, the extracellular vesicle is an exosome.

In some aspects, the composition is capable of being stored for at least about 4 hours, at least about 5 hours, at least about 6 hours, at least about 7 hours, at least about 8 hours, at least about 9 hours, at least about 10 hours, at least about 11 hours, at least about 12 hours, at least about 15 hours, at least about 20 hours, at least about 24 hours, at least about 2 days, at least about 3 days, at least about 4 days, at least about 5 days, at least about 6 days, or at least about 7 days at a temperature of 25° C.

In some aspects, the composition is capable of being frozen and thawed, wherein the thawed composition has a pH of about 7.2. In some aspects, the composition has a pH of 7.0, 7.1, 7.2, 7.3, or 7.4. In some aspects, the composition has a pH of 7.2. In some aspects, the pI is in the range of about 1 to about 6.5.

In some aspects, the composition has (i) reduced aggregates, (ii) improved stability of the EV, (iii) improved integrity of the EV architecture, and (iv) improved stability of engineered proteins contained on or in EVs.

In some aspects, the composition further comprises (i) a saccharide, (ii) sodium phosphate, (iii) potassium phosphate, (iv) sodium phosphate, or (v) any combination thereof.

In some aspects, the saccharide comprises a monosaccharide, a disaccharide, a trisaccharide, an oligosaccharide, a polysaccharide, a sugar alcohol, or any combination thereof. In some aspects, the saccharide has a molecular weight of from about 180.00 g/mol to about 380.00 g/mol. In some aspects, the saccharide comprises lactose, glucose, sucrose, trehalose, and/or combinations thereof. In some aspects, the saccharide is a sugar alcohol having a molecular weight of from about 90.00 g/mol to about 190.00 g/mol. In some aspects, the sugar alcohol comprises glycerol, sorbitol, mannitol, xylitol, and/or combinations thereof. In some aspects, the saccharide is a sucrose or a trehalose. In some aspects, the saccharide is present in the composition at a concentration of about 5% w/v.

In some aspects, the composition has a conductivity between about 6 mS/cm+/−10% and about 10 mS/cm+/−10%. In some aspects, the conductivity is between 6 mS/cm and about 7 mS/cm, between about 7 mS/cm and about 8 mS/cm, between about 8 mS/cm and about 9 mS/cm, or between about 9 mS/cm and about 10 mS/cm. In some aspects, the conductivity is about 6 mS/cm, about 7 mS/cm, about 8 mS/cm, about 9 mS/cm, or about 10 mS/cm.

In some aspects, the sodium chloride is present in the composition at a concentration of between about 50 mM and about 150 mM. In some aspects, the concentration of sodium chloride is between about 50 mM to about 140 mM, between about 60 mM to about 130 mM, between about 70 mM to about 120 mM, between about 80 mM to about 110 mM, between about 90 mM to about 100 mM, between about 100 mM to about 110 mM, between about 95 mM to about 105 mM, between about 95 mM to about 110 mM, or between about 90 mM to about 105 mM. In some aspects, the concentration of sodium chloride is about 50 mM, about 60 mM, about 70 mM, about 80 mM, about 90 mM, about 100 mM, about 110 mM, about 120 mM, about 130 mM, about 140 mM, or about 150 mM. In some aspects, the concentration of sodium chloride is about 95 mM, about 96 mM, about 97 mM, about 98 mM, about 99 mM, about 100 mM, about 101 mM, about 102 mM, about 103 mM, about 104 mM, or about 105 mM.

In some aspects, the potassium phosphate is present in the composition at a concentration of between about 1 mM to about 10 mM, between about 2 mM to about 9 mM, between about 3 mM to about 8 mM, between about 4 mM to about 7 mM, between about 5 mM to about 6 mM, or between about 4 mM to about 5 mM. In some aspects, the concentration of the potassium phosphate is about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, or about 5.5 mM. In some aspects, the concentration of the potassium phosphate is about 5 mM. In some aspects, the potassium phosphate is potassium phosphate monobasic.

In some aspects, the sodium phosphate is present in the composition at a concentration of between about 5 mM to about 30, between about 10 mM to about 20 mM, between about 11 mM to about 19 mM, between about 12 mM to about 18 mM, between about 13 mM to about 17 mM, between about 14 mM to about 16 mM, between about 15 mM to about 16 mM, or between about 14 mM to about 15 mM. In some aspects, the sodium phosphate is present in the composition at a concentration of about 14.5 mM, about 14.6 mM, about 14.7 mM, about 14.8 mM, about 14.9 mM, about 15.0 mM, about 15.1 mM about 15.2 mM, about 15.3 mM, about 15.4, mM, or about 15.5 mM. In some aspects, the concentration of the sodium phosphate is about 15 mM. In some aspects, the sodium phosphate is sodium phosphate dibasic heptahydrate.

In some aspects, the composition is not lyophilized. In some aspects, the composition does not comprise a chelating agent. In some aspects, the composition does not comprise albumin.

In some aspects, the composition comprises a sucrose at a concentration of about 5% w/v; sodium chloride at a concentration of about 100 mM; a potassium phosphate monobasic at a concentration of about 5 mM; a sodium phosphate dibasic heptahydrate at a concentration of about 15 mM; wherein the composition is in a solution at a pH of 7.2; and wherein the composition comprises an osmolarity of about 365 to about 425 mOsm/kg.

In some aspects, the composition comprises a sucrose at a concentration of about 5% w/v; sodium chloride at a concentration of about 100 mM; a potassium phosphate monobasic at a concentration of about 5 mM; a sodium phosphate dibasic heptahydrate at a concentration of about 15 mM; wherein the composition is in a solution at a pH of 7.2; and wherein the composition comprises an osmolarity of about 395 mOsm/kg.

In some aspects, the composition comprises a sucrose at a concentration of about 146 mM; sodium chloride at a concentration of about 100 mM; a potassium phosphate monobasic at a concentration of about 5 mM; a sodium phosphate dibasic heptahydrate at a concentration of about 15 mM; wherein the composition is in a solution at a pH of 7.2; and wherein the composition comprises an osmolarity of about 365 to about 425 mOsm/kg.

In some aspects, the composition comprises a sucrose at a concentration of about 146 mM; sodium chloride at a concentration of about 100 mM; a potassium phosphate monobasic at a concentration of about 5 mM; a sodium phosphate dibasic heptahydrate at a concentration of about 15 mM; wherein the composition is in a solution at a pH of 7.2; and wherein the composition comprises an osmolarity of about 395 mOsm/kg.

In some aspects, the composition is capable of being stored at a temperature of from about −20° C. to about −80° C., wherein the stability of the extracellular vesicle is not reduced. In some aspects, the composition can be stored for about one week, about two weeks, about three weeks, about four weeks, about one month, about two months, about three months, about four months, about five months, about six months, about seven months, about eight months, about nine months, about ten months, about 11 months, about 12 months, about one year, about two years, about three years, or about four years.

In some aspects, the extracellular vesicle further comprises a scaffold protein. In some aspects, the scaffold protein is Scaffold X.

In some aspects, a payload is linked to the scaffold protein. In some aspects, the payload is linked to the scaffold protein by a linker. In some aspects, the linker is a polypeptide. In some aspects, the linker is a non-polypeptide moiety.

In some aspects, Scaffold X is a scaffold protein that is capable of anchoring the payload on the exterior surface of the extracellular vesicle. In some aspects, the scaffold protein comprises prostaglandin F2 receptor negative regulator (the PTGFRN protein). In some aspects, the scaffold protein comprises the PTGFRN protein or a fragment thereof. In some aspects, the scaffold protein comprises an amino acid sequence as set forth in any one of SEQ ID NOs: 1 and 6-12. In some aspects, the scaffold protein comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 1.

In some aspects, the composition can be administered by a parenteral, topical, intravenous, oral, subcutaneous, intra-arterial, intradermal, transdermal, rectal, intracranial, intraperitoneal, intranasal, intratumoral, intramuscular route, or as an inhalant. In some aspects, the composition can be administered by an intrathecal route. In some aspects, the composition can be administered by a cerebroventricular route.

In some aspects, the ASO comprises a nucleic acid sequence selected from SEQ ID NOs: 91-193

In some aspects, the composition comprises (a) an extracellular vesicles comprising an ASO, wherein the ASO comprises a nucleic acid sequence selected from SEQ ID NOs: 91-193; (b) sucrose at a concentration of about 50 mM to about 150 mM; (c) sodium chloride at a concentration of about 100 mM to about 200 mM; (d) potassium phosphate monobasic at a concentration of about 5 mM; and (e) sodium phosphate dibasic at a concentration of about 15 mM, wherein the pH of the composition is about 7.2; and wherein the composition comprises an osmolarity of at least about 365 to about 425 mOsm/kg.

In some aspects, the composition comprises (a) an extracellular vesicles comprising an ASO, wherein the ASO comprises a nucleic acid sequence selected from SEQ ID NOs: 91-193; (b) sucrose at a concentration of about 4% to about 6%; (c) sodium chloride at a concentration of about 50 mM to about 150 mM; (d) potassium phosphate monobasic at a concentration of about 5 mM; (e) sodium phosphate dibasic at a concentration of about 15 mM, wherein the pH of the composition is about 7.2; and wherein the composition comprises an osmolarity of at least about 365 to about 425 mOsm/kg.

In some aspects, the composition comprises (a) extracellular vesicles comprising an ASO, wherein the ASO comprises a nucleic acid sequence selected from SEQ ID NOs: 91-193; (b) sucrose at a concentration of about 146 mM; (c) sodium chloride at a concentration of about 100 mM; (d) potassium phosphate monobasic at a concentration of about 5 mM; (e) sodium phosphate dibasic at a concentration of about 15 mM, wherein the pH of the composition is about 7.2; and wherein the composition comprises an osmolarity of at least about 365 to about 425 mOsm/kg.

In some aspects, the composition comprises (a) extracellular vesicles comprising an ASO, wherein the ASO comprises a nucleic acid sequence selected from SEQ ID NOs: 91-193; (b) sucrose at a concentration of about 5%; (c) sodium chloride at a concentration of about 100 mM; (d) potassium phosphate monobasic at a concentration of about 5 mM; (e) sodium phosphate dibasic at a concentration of about 15 mM, wherein the pH of the composition is about 7.2; and wherein the composition comprises an osmolarity of at least about 365 to about 425 mOsm/kg.

In some aspects, the composition comprises (a) extracellular vesicles comprising an ASO, wherein the ASO comprises a nucleic acid sequence selected from SEQ ID NOs: 91-193; (b) sucrose at a concentration of about 146 mM; (c) sodium chloride at a concentration of about 100 mM; (d) potassium phosphate monobasic at a concentration of about 5 mM; (e) sodium phosphate dibasic at a concentration of about 15 mM, wherein the pH of the composition is about 7.2; and wherein the composition comprises an osmolarity of about 395 mOsm/kg.

In some aspects, the composition comprises (a) extracellular vesicles comprising an ASO, wherein the ASO comprises a nucleic acid sequence selected from SEQ ID NOs: 91-193; (b) sucrose at a concentration of about 5%; (c) sodium chloride at a concentration of about 100 mM; (d) potassium phosphate monobasic at a concentration of about 5 mM; (e) sodium phosphate dibasic at a concentration of about 15 mM, wherein the pH of the composition is about 7.2; and wherein the composition comprises an osmolarity of about 395 mOsm/kg.

In some aspects, the extracellular vesicle is an exosome.

In some aspects, the ASO comprises the nucleic acid sequence set forth in SEQ ID NO: 144. In some aspects, the ASO comprises the nucleic acid sequence set forth in SEQ ID NO: 145. In some aspects, the ASO comprises the nucleic acid sequence set forth in SEQ ID NO: 193. In some aspects, the ASO comprises the nucleic acid sequence set forth in SEQ ID NO: 185.

In some aspects, the ASO is associated with the extracellular vesicles by a linker. In some aspects, the linker comprises cholesterol, tocopherol, a fatty acid, or any combination thereof. In some aspects, the linker is a cleavable linker. In some aspects, the linker is a cleavable linker.

Some aspects of the present disclosure are directed to a method of treating a disease or a condition in a subject in need thereof comprising administering to the subject a composition disclosed herein. In some aspects, the disease or condition is a cancer, a fibrosis, a hemophilia, diabetes, a growth factor deficiency, an eye disease, a Pompe disease, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, al antitrypsin deficiency, and a hypercholesterolemia. In some aspects, the cancer is bladder cancer, cervical cancer, renal cell cancer, testicular cancer, colorectal cancer, lung cancer, head and neck cancer, ovarian, lymphoma, liver cancer, glioblastoma, melanoma, myeloma, leukemia, pancreatic cancer, or combinations thereof.

Some aspects of the present disclosure are directed to a pharmaceutical composition disclosed herein for treating a disease or a condition in a subject in need thereof.

Some aspects of the present disclosure are directed to a use of a composition disclosed herein in the manufacture of a medicament for treating a disease or a condition.

Some aspects of the present disclosure are directed to a method of preparing a pharmaceutical composition disclosed herein comprising combining (i) an extracellular vesicle comprising an ASO and (ii) a salt or a saccharide; wherein the ASO comprises a contiguous nucleotide sequence of 10 to 30 nucleotides in length that is complementary to a nucleic acid sequence within a STAT6 transcript; and wherein the composition comprises an osmolarity lower than about 450 mOsm/kg. In some aspects, the salt comprises (a) sodium chloride; (b) a potassium phosphate; (c) a sodium phosphate, or (d) any combination thereof.

### BRIEF DESCRIPTION OF FIGURES

FIG. 1 is a table listing various ASO sequences that target the STAT6 transcript. The tables include the following information (from left to right): (i) description of the ASO, (ii) the ASO sequence without any particular design or chemical structure, (iii) SEQ ID number designated for the ASO sequence only, (iv) the ASO length, (v) the ASO sequence with a chemical structure, and (vi) the target start and end positions on the target transcript sequence (SEQ ID NO: 3). The ASOs are from 5′ to 3′. The symbols in the chemical structures are as follows: Nb means LNA; dN means DNA; 5MdC means 5-Methyl-dC; Nm means MOE; and s means phosphorothioate.

FIG. 2 is a schematic representation of a process for purifying exosomes loaded with an ASO.

FIGS. 3A-3C of ASO concentration (FIG. 3A), free ASO concentration (FIG. 3B), and IC50 (FIG. 3C) for sample stored up to 15 days at room temperature (RT) or at 5° C.

FIGS. 4A-4D are graphical representations of NTA (FIG. 4A), UV (FIG. 4B), AEX-UHPLC (FIG. 4C), and DLS (FIG. 4D) for CC700 filtered intermediates held at room temperature for 15 days.

FIGS. 5A-5D are graphical representations of NTA (FIG. 5A), UV (FIG. 5B), AEX-UHPLC (FIG. 5C), and DLS (FIG. 5D) for CC700 filtered intermediates held at 5° C. for 15 days.

FIG. 6A-6B are schematic representations of a process for evaluating loading temperature and NaCl/Sucrose in CC700 Column filtration.

### DETAILED DESCRIPTION OF DISCLOSURE

The present disclosure is directed to compositions comprising EVs, e.g., exosomes, comprising an antisense oligonucleotide (ASO), wherein the composition comprises an osmolarity of lower than about 450 mOsm/kg.

Non-limiting examples of the various aspects are disclosed herein.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payloads either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In some aspects, an EV comprises multiple (e.g., two or more) payloads or other exogenous biologically active moieties. In certain aspects, an extracellular vehicle can further comprise one or more scaffold moieties. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products. The EVs disclosed herein have been modified and therefore, do not comprise naturally occurring EVs.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In some aspects, an exosome comprises multiple (e.g., two or more) exogenous biologically active moieties (e.g., as described herein). In certain aspects, an exosome further comprises one or more scaffold moieties. As described infra, exosomes can be derived from a producer cell, and isolated from the producer cell based on its size, density, biochemical parameters, or a combination thereof. In some aspects, the EVs (e.g., exosomes) of the present disclosure are produced by cells that express one or more transgene products. The exosomes of the present disclosure are modified and therefore, do not comprise naturally occurring exosomes.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In some aspects, a nanovesicle comprises multiple (e.g., at least two) exogenous biologically active moieties. In certain aspects, a nanovesicle further comprises one or more scaffold moieties. Nanovesicles, once derived from a producer cell, can be isolated from the producer cell based on its size, density, biochemical parameters, or a combination thereof. As used herein, nanovesicles have been modified and therefore, do not comprise naturally occurring nanovesicles.

As used herein the term “surface-engineered EVs, e.g., exosomes” (e.g., Scaffold X-engineered EVs, e.g., exosomes) refers to an EV (e.g., exosome) with the membrane or the surface modified in its composition, so that the membrane or the surface of the engineered EV (e.g., exosome), is different from either that of the EV prior to the modification or of the naturally occurring EV. The engineering can be on the surface of the EV (e.g., exosome) or in the membrane of the EV (e.g., exosome) so that the surface of the EV, e.g., exosome, is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by genetic engineering or by being produced from a cell previously modified by genetic engineering. In some aspects, a surface-engineered EV, e.g., exosome, comprises multiple (e.g., at least two) exogenous biologically active moieties. In certain aspects, the exogenous biologically active moieties can comprise an exogenous protein (i.e., a protein that the EV, e.g., exosome, does not naturally express) or a fragment or variant thereof that can be exposed to the surface of the EV, e.g., exosome, or can be an anchoring point (attachment) for a moiety exposed on the surface of the EV, e.g., exosome. In other aspects, a surface-engineered EV, e.g., exosome, comprises a higher expression (e.g., higher number) of a natural exosome protein (e.g., Scaffold X) or a fragment or variant thereof that can be exposed to the surface of the EV, e.g., exosome, or can be an anchoring point (attachment) for a moiety exposed on the surface of the EV, e.g., exosome.

The term “modified,” when used in the context of EVs, e.g., exosomes described herein, refers to an alteration or engineering of an EV, e.g., exosome and/or its producer cell, such that the modified EV, e.g., exosome is different from a naturally-occurring EV, e.g., exosome. In some aspects, a modified EV, e.g., exosome described herein comprises a membrane that differs in composition of a protein, a lipid, a small molecular, a carbohydrate, etc. compared to the membrane of a naturally-occurring EV, e.g., exosome (e.g., membrane comprises higher density or number of natural exosome proteins and/or membrane comprises multiple (e.g., at least two) biologically active moieties that are not naturally found in exosomes. As used herein, biologically active moieties that are not naturally found in exosomes are also described as “exogenous biologically active moieties.” In certain aspects, such modifications to the membrane changes the exterior surface of the EV, e.g., exosome (e.g., surface-engineered EVs, e.g., exosomes described herein).

As used herein, the term “scaffold moiety” refers to a molecule that can be used to anchor a payload or any other exogenous biologically active moiety of interest to the EV, e.g., exosome, either on the luminal surface or on the exterior surface of the EV, e.g., exosome. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV, e.g., exosome. In some aspects, a scaffold moiety comprises a lipid, carbohydrate, or protein that does not naturally exist in the EV, e.g., exosome. In certain aspects, a scaffold moiety is Scaffold X. In further aspects, a scaffold moiety comprises a Scaffold X and another scaffold moiety. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor a moiety (e.g., a payload, e.g., an antisense oligonucleotide) to the external surface or the luminal surface of the exosome.

As used herein, the term “Scaffold Y” refers to exosome proteins that were newly identified within the luminal surface of exosomes. See, e.g., International Publication No. WO/2019/099942, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety on the luminal surface of the EVs, e.g., exosomes). In some aspects, a Scaffold Y can anchor a moiety (e.g., a sting agonist and/or an IL-12 moiety) to the lumen of the EVs, e.g., exosomes.

As used herein, the term “fragment” of a protein (e.g., therapeutic protein, or Scaffold X) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV, e.g., exosome. Whether a fragment is a functional fragment can be assessed by any known methods to determine the protein content of EVs, e.g., exosomes including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein.

As used herein, the term “variant” of a molecule (e.g., functional molecule, antigen, or Scaffold X) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

The generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten fold higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-la. They used random mutagenesis to generate over 3,500 individual IL-la mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X is modified at any convenient location.

As used herein the terms “linked to,” “conjugated to,” and “anchored to” are used interchangeably and refer to a covalent or non-covalent bond formed between a first moiety and a second moiety, e.g., Scaffold X and an exogenous biologically active moiety, respectively, e.g., a scaffold moiety expressed in or on the extracellular vesicle and an antigen, e.g., Scaffold X (e.g., a PTGFRN protein), respectively, in the luminal surface of or on the external surface of the extracellular vesicle. In some aspects, the first moeity is an ASO disclosed herein, and the second moeity is an anchoring moiety, e.g., a lipid, a steroid, or an analog or derivative thereof.

As used herein, the term “producer cell” refers to a cell used for generating an EV, e.g., exosome. A producer cell can be a cell cultured in vitro, or in a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP© amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof. In some aspects, the EVs, e.g., exosomes useful in the present disclosure do not carry an antigen on MHC class I or class II molecule exposed on the surface of the EV, e.g., exosome, but instead can carry an antigen in the lumen of the EV, e.g., exosome or on the surface of the EV, e.g., exosome by attachment to Scaffold X.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.99990%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “payload” refers to an agent that acts on a target (e.g., a target cell) that is contacted with the EV. A non-limiting example of a payload that can be included on the EV, e.g., exosome, is an ASO. Payloads that can be introduced into an EV, e.g., exosome, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an ASO.

As used herein, the term a “targeting moiety” refers to an agent that can modify the distribution of extracellular vesicles (e.g., exosomes, nanovesicles) in vivo or in vitro (e.g., in a mixed culture of cells of different varieties). The targeting moiety can be a biological molecule, such as a protein, a peptide, a lipid, or a carbohydrate, or a synthetic molecule. For example, the targeting moiety can be an antibody (e.g., anti-CD19 nanobody, anti-CD22 nanobody), a synthetic polymer (e.g., PEG), a natural ligand (e.g., CD40L, albumin), a recombinant protein (e.g., XTEN), but not limited thereto. In certain aspects, the targeting moiety is displayed on the surface of EVs. The targeting moiety can be displayed on the EV surface by being fused to a scaffold protein (e.g., Scaffold X) (e.g., as a genetically encoded fusion molecule). In some aspects, the targeting moiety can be displayed on the EV surface by chemical reaction attaching the targeting moiety to an EV surface molecule. A non-limiting example is PEGylation. In some aspects, EVs disclosed herein (e.g., exosomes) can further comprise a targeting moiety (in addition to a payload). In some aspects, a targeting moiety described above can be combined with a functional moiety, such as a small molecule (e.g., ASO), a drug, and/or a therapeutic protein (e.g., anti-CD3/anti-CD19 antibodies, anti-mesothelin antibody/pro-apoptotic proteins).

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. The term “antibody” includes, by way of example, both naturally occurring and non-naturally occurring antibodies; monoclonal and polyclonal antibodies; chimeric and humanized Abs; human or nonhuman Abs; wholly synthetic Abs; and single chain Abs. A nonhuman antibody may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

As used herein, the term “substantially free” means that the sample comprising EVs, e.g., exosomes, comprise less than 10% of macromolecules by mass/volume (m/v) percentage concentration. Some fractions can contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “conventional exosome protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

“Administering,” as used herein, means to give a composition comprising an EV, e.g., exosome, disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. EVs, e.g., exosomes can be administered as part of a pharmaceutical composition comprising at least one excipient.

An “effective amount” of, e.g., an extracellular vesicle as disclosed herein, is an amount sufficient to carry out a specifically stated purpose.

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

“Prevent” or “preventing,” as used herein, refers to decreasing or reducing the occurrence or severity of a particular outcome. In some aspects, preventing an outcome is achieved through prophylactic treatment.

### II. Pharmaceutical Compositions

Provided herein are compositions for the storage and administration of extracellular vesicles, e.g., exosomes, comprising an antisense oligonucleotide (ASO), wherein the composition comprises an osmolarity lower than about 450 mOsm/kg. In some aspects, the ASO comprises a contiguous nucleotide sequence of 10 to 30 nucleotides in length that is complementary to a nucleic acid sequence within a STAT6 transcript. As noted above, the compositions of the present disclosure provide multiple advantages, including but not limited to: reduced aggregation of EVs, improved stability of EVs, and improved integrity of EV architecture, improved stability of engineered proteins contained on or in EVs, and improved stability of loaded or conjugated materials such as an ASO. The compositions disclosed herein are capable of being frozen, stored at a range of temperatures over various lengths of time, and thawed, without compromising the stability of the EVs contained within the composition.

In some aspects, the osmolarity of the composition is at least about 300 to about 450 mOsm/kg. In some aspects, the osmolarity of the solution is at least about 310 to about 450 mOsm/kg, at least about 320 to about 450 mOsm/kg, at least about 330 to about 450 mOsm/kg, at least about 340 to about 450 mOsm/kg, at least about 350 to about 450 mOsm/kg, at least about 355 to about 450 mOsm/kg, at least about 360 to about 450 mOsm/kg, at least about 365 to about 450 mOsm/kg, at least about 365 to about 445 mOsm/kg, at least about 365 to about 440 mOsm/kg, at least about 365 to about 435 mOsm/kg, at least about 365 to about 430 mOsm/kg, at least about 365 to about 425 mOsm/kg, at least about 370 to about 420 mOsm/kg, at least about 375 to about 415 mOsm/kg, at least about 380 to about 410 mOsm/kg, at least about 385 to about 405 mOsm/kg, at least about 390 to about 400 mOsm/kg, at least about 395 to about 400 mOsm/kg, or at least about 390 to about 395 mOsm/kg. In some aspects, the osmolarity of the solution is at least about 365 to about 425 mOsm/kg. In some aspects, the osmolarity of the solution is at least about 300 mOsm/kg, about 310 mOsm/kg, about 320 mOsm/kg, about 330 mOsm/kg, about 340 mOsm/kg, about 350 mOsm/kg, about 360 mOsm/kg, about 365 mOsm/kg, about 370 mOsm/kg, about 375 mOsm/kg, about 380 mOsm/kg, about 385 mOsm/kg, about 390 mOsm/kg, about 395 mOsm/kg, about 400 mOsm/kg, about 405 mOsm/kg, about 410 mOsm/kg, about 415 mOsm/kg, about 420 mOsm/kg, about 425 mOsm/kg, about 430 mOsm/kg, or about 435 mOsm/kg, about 440 mOsm/kg, or about 445 mOsm/kg. In some aspects, the osmolarity of the solution is about 375 mOsm/kg. In some aspects, the osmolarity of the solution is about 380 mOsm/kg. In some aspects, the osmolarity of the solution is about 385 mOsm/kg. In some aspects, the osmolarity of the solution is about 390 mOsm/kg. In some aspects, the osmolarity of the solution is about 395 mOsm/kg. In some aspects, the osmolarity of the solution is about 400 mOsm/kg. In some aspects, the osmolarity of the solution is about 405 mOsm/kg. In some aspects, the osmolarity of the solution is about 410 mOsm/kg. In some aspects, the osmolarity of the solution is about 415 mOsm/kg. In some aspects, the osmolarity of the solution is about 420 mOsm/kg. In some aspects, the osmolarity of the solution is about 425 mOsm/kg.

In some aspects, the compositions further comprises a saccharide, and one or more salt. In some aspects, the salt comprises sodium chloride, a potassium phosphate, a sodium phosphate, or any combination thereof. In some aspects, the composition is in a solution at a pH of about 7.2.

The present disclosure provides a pharmaceutical composition comprising an extracellular vesicle, wherein the pharmaceutical composition is stable for freezing and/or storage and/or is suitable for administration in a mammal, e.g., human. Instability of a biologic during storage can be caused by aggregation, deamination, isomerization, hydrolysis, oxidation, and/or denaturation. These structural modifications can occur due to various different factors: the properties of the biologic and/or other factors including temperature, pH, and the ionic strength of the biologics and the elements formulated with the biologic.

In some aspects, the pharmaceutical composition of the present disclosure is formulated stable so that the composition does not require a chelating agent and/or albumin, e.g., recombinant human albumin.

Human albumin is the most ubiquitous protein in blood and is present at amounts around 40 g/L. Its role in blood is the shuttling of numerous smaller entities such as metals, hormones, fatty acids and toxins. However, it also makes up about 75% of the colloidal oncotic (or colloidal osmotic) pressure of blood and the single free cysteine of albumin (at position 34) makes up the majority of the reducing equivalents present in blood. All these properties are traits which are functional in employing albumin in formulation.

Albumin has historically been used in a range of different formulations. Originally, plasma sourced human serum albumin was employed, but there has been a shift in the industry towards the use of chemically defined (recombinant) human serum albumin. Recombinant products are advantageous due to factors such as: the absence of animal-derived products, certainty of supply, high purity, absence of host-derived proteases, high homogeneity, high free thiol content, absence of known or unknown human pathogens, batch-to-batch consistency and the presence of an established regulatory pathway.

Albumin in formulation has been reported to prevent: surface adsorption, aggregation, fibrillation, and oxidation and to improve: solubility, lyophilised cake formation, and/or dissolving properties of the API from lyophilised powder. Despite these known benefits, the present disclosure provides a stable albumin-free pharmaceutical composition comprising an extracellular vesicle.

Chelating agents are ingredients that bind with metal ions and play a crucial role in the stability and efficacy of pharmaceutical formulations. The process of chelation stabilizes metal ions by preventing them from chemically reacting with any other substances. The present composition can be characterized that it does not contain a chelating agent.

In some aspects, the compositions disclosed herein have a pI in the range of about 1 to about 6.5. The pI range of the presently disclosed EVs, where a surface macromolecule is over-expressed, e.g., PTGFRN, as disclosed herein, enables colloidally stable, anionic exosomes at physiological pH values. In some aspects, the surface molecule could be polypeptide, oligonucleotide, or carbohydrate. In some aspects, a pI above 6.5, can cause the EVs, e.g., exosomes to have either a neutral charge (unstable), or a cationic charge at useful pH ranges which could result in toxicity or limited biodistribution.

In some aspects, the compositions of the present disclosure are formulated in a liquid state and can be frozen for storage by way of reducing the temperature of the compositions to freezing and subfreezing temperatures. Freezing the compositions via dehydration or lyophilization is not contemplated. In some aspects, the composition is not lyophilized.

### II.A. Antisense Oligonucleotides (ASOs)

The present disclosure employs antisense oligonucleotides (ASOs) for use in modulating the function of nucleic acid molecules encoding mammalian STAT6, such as the STAT6 nucleic acid, e.g., STAT6 transcript, including STAT6 pre-mRNA, and STAT6 mRNA, or naturally occurring variants of such nucleic acid molecules encoding mammalian STAT6. The term “ASO” in the context of the present disclosure, refers to a molecule formed by covalent linkage of two or more nucleotides (i.e., an oligonucleotide).

In some aspects, the EV, e.g., the exosome, comprises at least one ASO. In some aspects, the EV, e.g., the exosome, comprises at least two ASOs, e.g., a first ASO comprising a first nucleotide sequence and a second ASO comprising a second nucleotide sequence. In some aspects, the EV, e.g., the exosome, comprises at least three ASOs, at least four ASOs, at least five ASOs, at least six ASOs, or more than six ASOs. In some aspects, each of the first ASO, the second ASO, the third ASO, the fourth ASO, the fifth ASO, the sixth ASO, and/or the ninth ASO is different.

In some aspects, the EV, e.g. the exosome, comprises a first ASO and a second ASO, wherein the first ASO comprises a first nucleotide sequence that is complimentary to a first target sequence in a first transcript, and wherein the second ASO comprises a second nucleotide sequence that is complimentary to a second target sequence in the first transcript. In some aspects, the first target sequence does not overlap with the second target sequence. In some aspects, the first target sequence comprises at least one nucleotide that is within the 5′UTR of the transcript, and the second target sequence does not comprise a nucleotide that is within the 5′UTR. In some aspects, the first target sequence comprises at least one nucleotide that is within the 3′UTR of the transcript, and the second target sequence does not comprise a nucleotide that is within the 3′UTR. In some aspects, the first target sequence comprises at least one nucleotide that is within the 5′UTR of the transcript, and the second target sequence comprises at least one nucleotide that is within the 3′UTR.

In some aspects, the first ASO targets a sequence within an exon-intron junction, and the second ASO targets a sequence within an exon-intron junction. In some aspects, the first ASO targets a sequence within an exon-intron junction, and the second ASO targets a sequence within an exon. In some aspects, the first ASO targets a sequence within an exon-intron junction, and the second ASO targets a sequence within an intron. In some aspects, the first ASO targets a sequence within an exon, and the second ASO targets a sequence within an exon. In some aspects, the first ASO targets a sequence within an intron, and the second ASO targets a sequence within an exon. In some aspects, the first ASO targets a sequence within an intron, and the second ASO targets a sequence within an intron.

In some aspects, the EV, e.g. the exosome, comprises a first ASO and a second ASO, wherein the first ASO comprises a first nucleotide sequence that is complimentary to a first target sequence in a first transcript, and wherein the second ASO comprises a second nucleotide sequence that is complimentary to a second target sequence in a second transcript, wherein the first transcript is not the product of the same gene as the second transcript.

The ASO comprises a contiguous nucleotide sequence of from about 10 to about 30, such as 10-20, 14-20, 16-20, or 15-25, nucleotides in length. In certain aspects, the ASO is 20 nucleotides in length. In certain aspects, the ASO is 18 nucleotides in length. In certain aspects, the ASO is 19 nucleotides in length. In certain aspects, the ASO is 17 nucleotides in length. In certain aspects, the ASO is 16 nucleotides in length. In certain aspects, the ASO is 15 nucleotides in length. In certain aspects, the ASO is 14 nucleotides in length. In certain aspects, the ASO is 13 nucleotides in length. In certain aspects, the ASO is 12 nucleotides in length. In certain aspects, the ASO is 11 nucleotides in length. In certain aspects, the ASO is 10 nucleotides in length.

In some aspects, the ASO comprises a contiguous nucleotide sequence of from about 10 to about 50 nucleotides in length, e.g., about 10 to about 45, about 10 to about 40, about 10 or about 35, or about 10 to about 30. In certain aspects, the ASO is 21 nucleotides in length. In certain aspects, the ASO is 22 nucleotides in length. In certain aspects, the ASO is 23 nucleotides in length. In certain aspects, the ASO is 24 nucleotides in length. In certain aspects, the ASO is 25 nucleotides in length. In certain aspects, the ASO is 26 nucleotides in length. In certain aspects, the ASO is 27 nucleotides in length. In certain aspects, the ASO is 28 nucleotides in length. In certain aspects, the ASO is 29 nucleotides in length. In certain aspects, the ASO is 30 nucleotides in length. In certain aspects, the ASO is 31 nucleotides in length. In certain aspects, the ASO is 32 nucleotides in length. In certain aspects, the ASO is 33 nucleotides in length. In certain aspects, the ASO is 34 nucleotides in length. In certain aspects, the ASO is 35 nucleotides in length. In certain aspects, the ASO is 36 nucleotides in length. In certain aspects, the ASO is 37 nucleotides in length. In certain aspects, the ASO is 38 nucleotides in length. In certain aspects, the ASO is 39 nucleotides in length. In certain aspects, the ASO is 40 nucleotides in length. In certain aspects, the ASO is 41 nucleotides in length. In certain aspects, the ASO is 42 nucleotides in length. In certain aspects, the ASO is 43 nucleotides in length. In certain aspects, the ASO is 44 nucleotides in length. In certain aspects, the ASO is 45 nucleotides in length. In certain aspects, the ASO is 46 nucleotides in length. In certain aspects, the ASO is 47 nucleotides in length. In certain aspects, the ASO is 48 nucleotides in length. In certain aspects, the ASO is 49 nucleotides in length. In certain aspects, the ASO is 50 nucleotides in length.

The terms “antisense ASO,” “antisense oligonucleotide,” and “oligomer” as used herein are interchangeable with the term “ASO.”

A reference to a SEQ ID number includes a particular nucleobase sequence, but does not include any design or full chemical structure. Furthermore, the ASOs disclosed in the figures herein show a representative design, but are not limited to the specific design shown in the figures unless otherwise indicated. For example, when a claim (or this specification) refers to SEQ ID NO: 101, it includes the nucleotide sequence of SEQ ID NO: 101 only. The design of any ASO disclosed herein can be written as SEQ ID NO: XX, wherein each of the first nucleotide, the second nucleotide, the third nucleotide, the first nucleotide, the second nucleotide, and the Nth nucleotide from the 5′ end is a modified nucleotide, e.g., LNA, and each of the other nucleotides is a non-modified nucleotide (e.g., DNA).

In various aspects, the ASO of the disclosure does not comprise RNA (units). In some aspects, the ASO comprises one or more DNA units. In one aspect, the ASO according to the disclosure is a linear molecule or is synthesized as a linear molecule. In some aspects, the ASO is a single stranded molecule, and does not comprise short regions of, for example, at least 3, 4 or 5 contiguous nucleotides, which are complementary to equivalent regions within the same ASO (i.e. duplexes)—in this regard, the ASO is not (essentially) double stranded. In some aspects, the ASO is essentially not double stranded. In some aspects, the ASO is not a siRNA. In various aspects, the ASO of the disclosure can consist entirely of the contiguous nucleotide region. Thus, in some aspects the ASO is not substantially self-complementary.

In other aspects, the present disclosure includes fragments of ASOs. For example, the disclosure includes at least one nucleotide, at least two contiguous nucleotides, at least three contiguous nucleotides, at least four contiguous nucleotides, at least five contiguous nucleotides, at least six contiguous nucleotides, at least seven contiguous nucleotides, at least eight contiguous nucleotides, or at least nine contiguous nucleotides of the ASOs disclosed herein. Fragments of any of the sequences disclosed herein are contemplated as part of the disclosure.

In some aspects, the ASOs for the present disclosure include a phosphorodiamidate Morpholino oligomer (PMO) or a peptide-conjugated phosphorodiamidate morpholino oligomer (PPMO).

### II.A.1. ASOs Targeting STAT6

Suitably the ASO of the disclosure is capable of down-regulating (e.g., reducing or removing) expression of the STAT6 mRNA or STAT6 protein. In this regard, the ASO of the disclosure can promote differentiation of M2 macrophages and/or decrease the differentiation of M1 macrophages. In particular, the present disclosure is directed to ASOs that target one or more regions of the STAT6 pre-mRNA (e.g., intron regions, exon regions, and/or exon-intron junction regions).

Unless indicated otherwise, the term “STAT6,” as used herein, can refer to STAT6 from one or more species (e.g., humans, non-human primates, dogs, cats, guinea pigs, rabbits, rats, mice, horses, cattle, and bears).

STAT6 (STAT6) is also known as signal transducer and activator of transcription 6. Synonyms of STAT6/STAT6 are known and include IL-4 STAT; STAT, Interleukin4-Induced; Transcription Factor IL-4 STAT; STAT6B; STAT6C; and D12S1644. The sequence for the human STAT6 gene can be found under publicly available GenBank Accession Number NC_000012.12:c57111413-57095404. The human STAT6 gene is found at chromosome location 12q13.3 at 57111413-57095404, complement.

The sequence for the human STAT6 pre-mRNA transcript (SEQ ID NO: 1) corresponds to the reverse complement of residues 57111413-57095404, complement, of chromosome 12q13.3. The STAT6 mRNA sequence (GenBank Accession No. NM_001178078.1) is provided in SEQ ID NO: 3 (Table 1), except that the nucleotide “t” in SEQ ID NO: 3 is shown as “u” in the mRNA. The sequence for human STAT6 protein can be found under publicly available Accession Numbers: P42226-1, (canonical sequence, SEQ ID NO: 2; Table 1), P42226-2 (SEQ ID NO: 4), and P42226-3 (SEQ ID NO: 5), each of which is incorporated by reference herein in its entirety.

TABLE 1
STAT6 mRNA and Protein Sequences

STAT6 mRNA Sequence
GGGGCAGCCACTGCTTACACTGAAGAGGGAGGACGGGAGAGGAGTGTGTGTGTGTGTGTGTGTGTGTGTGTGTATG
TATGTGTGTGCTTTATCTTATTTTTCTTTTTGGTGGTGGTGGTGGAAGGGGGGAGGTGCTAGCAGGGCCAGCCTTG
AACTCGCTGGACAGAGCTACAGACCTATGGGGCCTGGAAGTGCCCGCTGAGAAAGGGAGAAGACAGCAGAGGGGTT
GCCGAGGCAACCTCCAAGTCCCAGATCATGTCTCTGTGGGGTCTGGTCTCCAAGATGCCCCCAGAAAAAGTGCAGC
GGCTCTATGTCGACTTTCCCCAACACCTGCGGCATCTTCTGGGTGACTGGCTGGAGAGCCAGCCCTGGGAGTTCCT
GGTCGGCTCCGACGCCTTCTGCTGCAACTTGGCTAGTGCCCTACTTTCAGACACTGTCCAGCACCTTCAGGCCTCG
GTGGGAGAGCAGGGGGAGGGGAGCACCATCTTGCAACACATCAGCACCCTTGAGAGCATATATCAGAGGGACCCCC
TGAAGCTGGTGGCCACTTTCAGACAAATACTTCAAGGAGAGAAAAAAGCTGTTATGGAACAGTTCCGCCACTTGCC
AATGCCTTTCCACTGGAAGCAGGAAGAACTCAAGTTTAAGACAGGCTTGCGGAGGCTGCAGCACCGAGTAGGGGAG
ATCCACCTTCTCCGAGAAGCCCTGCAGAAGGGGGCTGAGGCTGGCCAAGTGTCTCTGCACAGCTTGATAGAAACTC
CTGCTAATGGGACTGGGCCAAGTGAGGCCCTGGCCATGCTACTGCAGGAGACCACTGGAGAGCTAGAGGCAGCCAA
AGCCCTAGTGCTGAAGAGGATCCAGATTTGGAAACGGCAGCAGCAGCTGGCAGGGAATGGCGCACCGTTTGAGGAG
AGCCTGGCCCCACTCCAGGAGAGGTGTGAAAGCCTGGTGGACATTTATTCCCAGCTACAGCAGGAGGTAGGGGCGG
CTGGTGGGGAGCTTGAGCCCAAGACCCGGGCATCGCTGACTGGCCGGCTGGATGAAGTCCTGAGAACCCTCGTCAC
CAGTTGCTTCCTGGTGGAGAAGCAGCCCCCCCAGGTACTGAAGACTCAGACCAAGTTCCAGGCTGGAGTTCGATTC
CTGTTGGGCTTGAGGTTCCTGGGGGCCCCAGCCAAGCCTCCGCTGGTCAGGGCCGACATGGTGACAGAGAAGCAGG
CGCGGGAGCTGAGTGTGCCTCAGGGTCCTGGGGCTGGAGCAGAAAGCACTGGAGAAATCATCAACAACACTGTGCC
CTTGGAGAACAGCATTCCTGGGAACTGCTGCTCTGCCCTGTTCAAGAACCTGCTTCTCAAGAAGATCAAGCGGTGT
GAGCGGAAGGGCACTGAGTCTGTCACAGAGGAGAAGTGCGCTGTGCTCTTCTCTGCCAGCTTCACACTTGGCCCCG
GCAAACTCCCCATCCAGCTCCAGGCCCTGTCTCTGCCCCTGGTGGTCATCGTCCATGGCAACCAAGACAACAATGC
CAAAGCCACTATCCTGTGGGACAATGCCTTCTCTGAGATGGACCGCGTGCCCTTTGTGGTGGCTGAGCGGGTGCCC
TGGGAGAAGATGTGTGAAACTCTGAACCTGAAGTTCATGGCTGAGGTGGGGACCAACCGGGGGCTGCTCCCAGAGC
ACTTCCTCTTCCTGGCCCAGAAGATCTTCAATGACAACAGCCTCAGTATGGAGGCCTTCCAGCACCGTTCTGTGTC
CTGGTCGCAGTTCAACAAGGAGATCCTGCTGGGCCGTGGCTTCACCTTTTGGCAGTGGTTTGATGGTGTCCTGGAC
CTCACCAAACGCTGTCTCCGGAGCTACTGGTCTGACCGGCTGATCATTGGCTTCATCAGCAAACAGTACGTTACTA
GCCTTCTTCTCAATGAGCCCGACGGAACCTTTCTCCTCCGCTTCAGCGACTCAGAGATTGGGGGCATCACCATTGC
CCATGTCATCCGGGGCCAGGATGGCTCTCCACAGATAGAGAACATCCAGCCATTCTCTGCCAAAGACCTGTCCATT
CGCTCACTGGGGGACCGAATCCGGGATCTTGCTCAGCTCAAAAATCTCTATCCCAAGAAGCCCAAGGATGAGGCTT
TCCGGAGCCACTACAAGCCTGAACAGATGGGTAAGGATGGCAGGGGTTATGTCCCAGCTACCATCAAGATGACCGT
GGAAAGGGACCAACCACTTCCTACCCCAGAGCTCCAGATGCCTACCATGGTGCCTTCTTATGACCTTGGAATGGCC
CCTGATTCCTCCATGAGCATGCAGCTTGGCCCAGATATGGTGCCCCAGGTGTACCCACCACACTCTCACTCCATCC
CCCCGTATCAAGGCCTCTCCCCAGAAGAATCAGTCAACGTGTTGTCAGCCTTCCAGGAGCCTCACCTGCAGATGCC
CCCCAGCCTGGGCCAGATGAGCCTGCCCTTTGACCAGCCTCACCCCCAGGGCCTGCTGCCGTGCCAGCCTCAGGAG
CATGCTGTGTCCAGCCCTGACCCCCTGCTCTGCTCAGATGTGACCATGGTGGAAGACAGCTGCCTGAGCCAGCCAG
TGACAGCGTTTCCTCAGGGCACTTGGATTGGTGAAGACATATTCCCTCCTCTGCTGCCTCCCACTGAACAGGACCT
CACTAAGCTTCTCCTGGAGGGGCAAGGGGAGTCGGGGGGAGGGTCCTTGGGGGCACAGCCCCTCCTGCAGCCCTCC
CACTATGGGCAATCTGGGATCTCAATGTCCCACATGGACCTAAGGGCCAACCCCAGTTGGTGATCCCAGCTGGAGG
GAGAACCCAAAGAGACAGCTCTTCTACTACCCCCACAGACCTGCTCTGGACACTTGCTCATGCCCTGCCAAGCAGC
AGATGGGGAGGGTGCCCTCCTATCCCCACCTACTCCTGGGTCAGGAGGAAAAGACTAACAGGAGAATGCACAGTGG
GTGGAGCCAATCCACTCCTTCCTTTCTATCATTCCCCTGCCCACCTCCTTCCAGCACTGACTGGAAGGGAAGTTCA
GGCTCTGAGACACACCCCAACATGCCTGCACCTGCAGCGCGCACACGCACGCACACACACATACAGAGCTCTCTGA
GGGTGATGGGGCTGAGCAGGAGGGGGGCTGGGTAAGAGCACAGGTTAGGGCATGGAAGGCTTCTCCGCCCATTCTG
ACCCAGGGCCTAGGACGGATAGGCAGGAACATACAGACACATTTACACTAGAGGCCAGGGATAGAGGATATTGGGT
CTCAGCCCTAGGGGAATGGGAAGCAGCTCAAGGGACCCTGGGTGGGAGCATAGGAGGGGTCTGGACATGTGGTTAC
TAGTACAGGTTTTGCCCTGATTAAAAAATCTCCCAAAGCCCCAAATTCCTGTTAGCCAGGTGGAGGCTTCTGATAC
GTGTATGAGACTATGCAAAAGTACAAGGGCTGAGATTCTTCGTGTATAGCTGTGTGAACGTGTATGTACCTAGGAT
ATGTTAAATGTATAGCTGGCACCTTAGTTGCATGACCACATAGAACATGTGTCTATCTGCTTTTGCCTACGTGACA
ACACAAATTTGGGAGGGTGAGACACTGCACAGAAGACAGCAGCAAGTGTGCTGGCCTCTCTGACATATGCTAACCC
CCAAATACTCTGAATTTGGAGTCTGACTGTGCCCAAGTGGGTCCAAGTGGCTGTGACATCTACGTATGGCTCCACA
CCTCCAATGCTGCCTGGGAGCCAGGGTGAGAGTCTGGGTCCAGGCCTGGCCATGTGGCCCTCCAGTGTATGAGAGG
GCCCTGCCTGCTGCATCTTTTCTGTTGCCCCATCCACCGCCAGCTTCCCTTCACTCCCCTATCCCATTCTCCCTCT
CAAGGCAGGGGTCATAGATCCTAAGCCATAAAATAAATTTTATTCCAAAATAACAAAATAAATAATCTACTGTACA
CAATCTGAAAA (SEQ ID NO: 3)
STAT6 Protein Sequence
MSLWGLVSKMPPEKVQRLYVDFPQHLRHLLGDWLESQPWEFLVGSDAFCCNLASALLSDTVQHLQASVGEQGEGST
ILQHISTLESIYORDPLKLVATFRQILQGEKKAVMEQFRHLPMPFHWKQEELKFKTGLRRLQHRVGEIHLLREALQ
KGAEAGQVSLHSLIETPANGTGPSEALAMLLQETTGELEAAKALVLKRIQIWKRQQQLAGNGAPFEESLAPLQERC
ESLVDIYSQLQQEVGAAGGELEPKTRASLTGRLDEVLRTLVTSCFLVEKQPPQVLKTQTKFQAGVRFLLGLRFLGA
PAKPPLVRADMVTEKQARELSVPQGPGAGAESTGEIINNTVPLENSIPGNCCSALFKNLLLKKIKRCERKGTESVT
EEKCAVLFSASFTLGPGKLPIQLQALSLPLVVIVHGNQDNNAKATILWDNAFSEMDRVPFVVAERVPWEKMCETLN
LKFMAEVGTNRGLLPEHFLFLAQKIFNDNSLSMEAFQHRSVSWSQFNKEILLGRGFTFWQWFDGVLDLTKRCLRSY
WSDRLIIGFISKQYVTSLLLNEPDGTFLLRFSDSEIGGITIAHVIRGODGSPQIENIQPFSAKDLSIRSLGDRIRD
LAQLKNLYPKKPKDEAFRSHYKPEQMGKDGRGYVPATIKMTVERDQPLPTPELQMPTMVPSYDLGMAPDSSMSMQL
GPDMVPQVYPPHSHSIPPYQGLSPEESVNVLSAFQEPHLQMPPSLGQMSLPFDQPHPQGLLPCQPQEHAVSSPDPL
LCSDVTMVEDSCLSQPVTAFPQGTWIGEDIFPPLLPPTEQDLTKLLLEGQGESGGGSLGAQPLLQPSHYGQSGISM
SHMDLRANPSW (SEQ ID NO: 2)

Natural variants of the human STAT6 gene product are known. For example, natural variants of human STAT6 protein can contain one or more amino acid substitutions selected from: M118R, D419N, and any combination thereof. Additional variants of human STAT6 protein resulting from alternative splicing are also known in the art. STAT6 Isoform 2 (identifier: P42226-2 at UniProt) differs from the canonical sequence (SEQ ID NO: 3) as follows: deletion of residues 1-174 and substitution of 175PSE177 with 1751WQ177 relative to SEQ ID NO: 3. The sequence of STAT6 Isoform 3 (identifier: P42226-3) differs from the canonical sequence (SEQ ID NO: 3) as follows: deletion of residues 1-110 relative to SEQ ID NO: 3. Therefore, the ASOs of the present disclosure can be designed to reduce or inhibit expression of the natural variants of the STAT6 protein.

An example of a target nucleic acid sequence of the ASOs is STAT6 pre-mRNA. SEQ ID NO: 1 represents a human STAT6 genomic sequence (i.e., reverse complement of nucleotides 57111413-57095404, complement, of chromosome 12q13.3). SEQ ID NO: 1 is identical to a STAT6 pre-mRNA sequence except that nucleotide “t” in SEQ TD NO: 1 is shown as “u” in pre-mRNA. In certain aspects, the “target nucleic acid” comprises an intron of a STAT6 protein-encoding nucleic acids or naturally occurring variants thereof, and RNA nucleic acids derived therefrom, e.g., pre-mRNA. In other aspects, the target nucleic acid comprises an exon region of a STAT6 protein-encoding nucleic acids or naturally occurring variants thereof, and RNA nucleic acids derived therefrom, e.g., pre-mRNA. In yet other aspects, the target nucleic acid comprises an exon-intron junction of a STAT6 protein-encoding nucleic acids or naturally occurring variants thereof, and RNA nucleic acids derived therefrom, e.g., pre-mRNA. In some aspects, for example when used in research or diagnostics the “target nucleic acid” can be a cDNA or a synthetic oligonucleotide derived from the above DNA or RNA nucleic acid targets. The human STAT6 protein sequence encoded by the STAT6 pre-mRNA is shown as SEQ ID NO: 3. In other aspects, the target nucleic acid comprises an untranslated region of a STAT6 protein-encoding nucleic acids or naturally occurring variants thereof, e.g., 5′ UTR, 3′ UTR, or both.

In some aspects, an ASO of the disclosure hybridizes to a region within the introns of a STAT6 transcript, e.g., SEQ ID NO: 1. In certain aspects, an ASO of the disclosure hybridizes to a region within the exons of a STAT6 transcript, e.g., SEQ ID NO: 1. In other aspects, an ASO of the disclosure hybridizes to a region within the exon-intron junction of a STAT6 transcript, e.g., SEQ ID NO: 1. In some aspects, an ASO of the disclosure hybridizes to a region within a STAT6 transcript (e.g., an intron, exon, or exon-intron junction), e.g., SEQ ID NO: 1, wherein the ASO has a design according to formula: 5′ A-B-C 3′ as described elsewhere herein.

In some aspects, the ASO targets a mRNA encoding a particular isoform of STAT6 protein (e.g., Isoform 1). In some aspects, the ASO targets all isoforms of STAT6 protein. In other aspects, the ASO targets two isoforms (e.g., Isoform 1 and Isoform 2, Isoform 1 and Isoform 3, or Isoform 2 and Isoform 3) of STAT6 protein.

In some aspects, the ASO comprises a contiguous nucleotide sequence (e.g., 10 to 30 nucleotides in length, e.g., 20 nucleotides in length) that are complementary to a nucleic acid sequence within a STAT6 transcript, e.g., a region corresponding to SEQ ID NO: 1 or SEQ ID NO: 3. In some aspects, the ASO comprises a contiguous nucleotide sequence that hybridizes to a nucleic acid sequence, or a region within the sequence, of a STAT6 transcript (“target region”), wherein the nucleic acid sequence corresponds (i) nucleotides 1-700 of SEQ ID NO: 3; (ii) nucleotides 1000-1500 of SEQ ID NO: 3; (iii) nucleotides 1500-2000 of SEQ ID NO: 3; (iv) nucleotides 2000-2500 of SEQ ID NO: 3; (v) 2500-3000 of SEQ ID NO: 3; or (vi) 3000-3700 of SEQ ID NO: 3 and wherein, optionally, the ASO has one of the designs described herein or a chemical structure shown elsewhere herein.

In some aspects, the ASO comprises a contiguous nucleotide sequence that hybridizes to a nucleic acid sequence, or a region within the sequence, of a STAT6 transcript (“target region”), wherein the nucleic acid sequence corresponds to (i) nucleotides 413-803 of SEQ ID NO: 3; (ii) nucleotides 952-1688 of SEQ ID NO: 3; (iii) nucleotides 1726-2489 of SEQ ID NO: 3; (iv) nucleotides 2682-2912 of SEQ ID NO: 3; (v) 2970-3203 of SEQ ID NO: 3; or (vi) 3331-3561 of SEQ ID NO: 3 and wherein, optionally, the ASO has one of the designs described herein or a chemical structure shown elsewhere herein.

In some aspects, the ASO comprises a contiguous nucleotide sequence that hybridizes to a nucleic acid sequence, or a region within the sequence, of a STAT6 transcript (“target region”), wherein the nucleic acid sequence corresponds to (i) nucleotides 463-753 of SEQ ID NO: 3; (ii) nucleotides 1002-1638 of SEQ ID NO: 3; (iii) nucleotides 1776-2439 of SEQ ID NO: 3; (iv) nucleotides 2682-2862 of SEQ ID NO: 3; (v) 3020-3153 of SEQ ID NO: 3; or (vi) 3381-3511 of SEQ ID NO: 3 and wherein, optionally, the ASO has one of the designs described herein or a chemical structure shown elsewhere herein.

In some aspects, the ASO comprises a contiguous nucleotide sequence that hybridizes to a nucleic acid sequence, or a region within the sequence, of a STAT6 transcript (“target region”), wherein the nucleic acid sequence corresponds to (i) nucleotides 503-713 of SEQ ID NO: 3; (ii) nucleotides 1042-1598 of SEQ ID NO: 3; (iii) nucleotides 1816-2399 of SEQ ID NO: 3; (iv) nucleotides 2722-2822 of SEQ ID NO: 3; (v) 3060-3113 of SEQ ID NO: 3; or (vi) 3421-3471 of SEQ ID NO: 3 and wherein, optionally, the ASO has one of the designs described herein or a chemical structure shown elsewhere herein.

In some aspects, the target region corresponds to nucleotides 1053-1067 of SEQ ID NO: 3 (e.g., ASO-STAT6-1053; SEQ ID NO: 91). In some aspects, the target region corresponds to nucleotides 1359-1373 of SEQ ID NO: 3 (e.g., ASO-STAT6-1359; SEQ ID NO: 92). In some aspects, the target region corresponds to nucleotides 1890-1904 of SEQ ID NO: 3 (e.g., ASO-STAT6-1890; SEQ ID NO: 93). In some aspects, the target region corresponds to nucleotides 1892-1906 of SEQ ID NO: 3 (e.g., ASO-STAT6-1892; SEQ ID NO: 94). In some aspects, the target region corresponds to nucleotides 1915-1929 of SEQ ID NO: 3 (e.g., ASO-STAT6-1915; SEQ ID NO: 95). In some aspects, the target region corresponds to nucleotides 1916-1930 of SEQ ID NO: 3 (e.g., ASO-STAT6-1916; SEQ ID NO: 96). In some aspects, the target region corresponds to nucleotides 1917-1931 of SEQ ID NO: 3 (e.g., ASO-STAT6-1917; SEQ ID NO: 97). In some aspects, the target region corresponds to nucleotides 1918-1932 of SEQ ID NO: 3 (e.g., ASO-STAT6-1918; SEQ ID NO: 98). In some aspects, the target region corresponds to nucleotides 1919-1933 of SEQ ID NO: 3 (e.g., ASO-STAT6-1919; SEQ ID NO: 99). In some aspects, the target region corresponds to nucleotides 1920-1934 of SEQ ID NO: 3 (e.g., ASO-STAT6-1920; SEQ ID NO: 100). In some aspects, the target region corresponds to nucleotides 1937-1951 of SEQ ID NO: 3 (e.g., ASO-STAT6-1937; SEQ ID NO: 101). In some aspects, the target region corresponds to nucleotides 1938-1952 of SEQ ID NO: 3 (e.g., ASO-STAT6-1938; SEQ ID NO: 102). In some aspects, the target region corresponds to nucleotides 2061-2075 of SEQ ID NO: 3 (e.g., ASO-STAT6-2061; SEQ ID NO: 103). In some aspects, the target region corresponds to nucleotides 2062-2076 of SEQ ID NO: 3 (e.g., ASO-STAT6-2062; SEQ ID NO: 104). In some aspects, the target region corresponds to nucleotides 2063-2077 of SEQ ID NO: 3 (e.g., ASO-STAT6-2063; SEQ ID NO: 105). In some aspects, the target region corresponds to nucleotides 2064-2078 of SEQ ID NO: 3 (e.g., ASO-STAT6-2064; SEQ ID NO: 106). In some aspects, the target region corresponds to nucleotides 2066-2080 of SEQ ID NO: 3 (e.g., ASO-STAT6-2066; SEQ ID NO: 107). In some aspects, the target region corresponds to nucleotides 2067-2081 of SEQ ID NO: 3 (e.g., ASO-STAT6-2067; SEQ ID NO: 108). In some aspects, the target region corresponds to nucleotides 2068-2082 of SEQ ID NO: 3 (e.g., ASO-STAT6-2068; SEQ ID NO: 109). In some aspects, the target region corresponds to nucleotides 2352-2366 of SEQ ID NO: 3 (e.g., ASO-STAT6-2352; SEQ ID NO: 110). In some aspects, the target region corresponds to nucleotides 3073-3087 of SEQ ID NO: 3 (e.g., ASO-STAT6-3073; SEQ ID NO: 111). In some aspects, the target region corresponds to nucleotides 1053-1068 of SEQ ID NO: 3 (e.g., ASO-STAT6-1053; SEQ ID NO: 112). In some aspects, the target region corresponds to nucleotides 1054-1069 of SEQ ID NO: 3 (e.g., ASO-STAT6-1054; SEQ ID NO: 113). In some aspects, the target region corresponds to nucleotides 1356-1371 of SEQ ID NO: 3 (e.g., ASO-STAT6-1356; SEQ ID NO: 114). In some aspects, the target region corresponds to nucleotides 1847-1862 of SEQ ID NO: 3 (e.g., ASO-STAT6-1847; SEQ ID NO: 115). In some aspects, the target region corresponds to nucleotides 1886-1901 of SEQ ID NO: 3 (e.g., ASO-STAT6-1886; SEQ ID NO: 116). In some aspects, the target region corresponds to nucleotides 1887-1902 of SEQ ID NO: 3 (e.g., ASO-STAT6-1887; SEQ ID NO: 117). In some aspects, the target region corresponds to nucleotides 1888-1903 of SEQ ID NO: 3 (e.g., ASO-STAT6-1888; SEQ ID NO: 118). In some aspects, the target region corresponds to nucleotides 1889-1904 of SEQ ID NO: 3 (e.g., ASO-STAT6-1889; SEQ ID NO: 119). In some aspects, the target region corresponds to nucleotides 1890-1905 of SEQ ID NO: 3 (e.g., ASO-STAT6-1890; SEQ ID NO: 120). In some aspects, the target region corresponds to nucleotides 1893-1908 of SEQ ID NO: 3 (e.g., ASO-STAT6-1893; SEQ ID NO: 121). In some aspects, the target region corresponds to nucleotides 1917-1932 of SEQ ID NO: 3 (e.g., ASO-STAT6-1917; SEQ ID NO: 122). In some aspects, the target region corresponds to nucleotides 1919-1934 of SEQ ID NO: 3 (e.g., ASO-STAT6-1919; SEQ ID NO: 123). In some aspects, the target region corresponds to nucleotides 2056-2071 of SEQ ID NO: 3 (e.g., ASO-STAT6-2056; SEQ ID NO: 124). In some aspects, the target region corresponds to nucleotides 2060-2075 of SEQ ID NO: 3 (e.g., ASO-STAT6-2060; SEQ ID NO: 125). In some aspects, the target region corresponds to nucleotides 2066-2081 of SEQ ID NO: 3 (e.g., ASO-STAT6-2066; SEQ ID NO: 126). In some aspects, the target region corresponds to nucleotides 2070-2085 of SEQ ID NO: 3 (e.g., ASO-STAT6-2070; SEQ ID NO: 127). In some aspects, the target region corresponds to nucleotides 2351-2366 of SEQ ID NO: 3 (e.g., ASO-STAT6-2351; SEQ ID NO: 128). In some aspects, the target region corresponds to nucleotides 2352-2367 of SEQ ID NO: 3 (e.g., ASO-STAT6-2352; SEQ ID NO: 129). In some aspects, the target region corresponds to nucleotides 2359-2374 of SEQ ID NO: 3 (e.g., ASO-STAT6-2359; SEQ ID NO: 130). In some aspects, the target region corresponds to nucleotides 3633-3648 of SEQ ID NO: 3 (e.g., ASO-STAT6-3633; SEQ ID NO: 131). In some aspects, the target region corresponds to nucleotides 673-689 of SEQ ID NO: 3 (e.g., ASO-STAT6-673; SEQ ID NO: 132). In some aspects, the target region corresponds to nucleotides 1052-1068 of SEQ ID NO: 3 (e.g., ASO-STAT6-1052; SEQ ID NO: 133). In some aspects, the target region corresponds to nucleotides 1356-1372 of SEQ ID NO: 3 (e.g., ASO-STAT6-1356; SEQ ID NO: 134). In some aspects, the target region corresponds to nucleotides 1357-1373 of SEQ ID NO: 3 (e.g., ASO-STAT6-1357; SEQ ID NO: 135). In some aspects, the target region corresponds to nucleotides 1359-1375 of SEQ ID NO: 3 (e.g., ASO-STAT6-1359; SEQ ID NO: 136). In some aspects, the target region corresponds to nucleotides 1360-1376 of SEQ ID NO: 3 (e.g., ASO-STAT6-1360; SEQ ID NO: 137). In some aspects, the target region corresponds to nucleotides 1839-1855 of SEQ ID NO: 3 (e.g., ASO-STAT6-1839; SEQ ID NO: 138). In some aspects, the target region corresponds to nucleotides 1848-1864 of SEQ ID NO: 3 (e.g., ASO-STAT6-1848; SEQ ID NO: 139). In some aspects, the target region corresponds to nucleotides 1849-1865 of SEQ ID NO: 3 (e.g., ASO-STAT6-1849; SEQ ID NO: 140). In some aspects, the target region corresponds to nucleotides 1891-1907 of SEQ ID NO: 3 (e.g., ASO-STAT6-1891; SEQ ID NO: 141). In some aspects, the target region corresponds to nucleotides 1915-1931 of SEQ ID NO: 3 (e.g., ASO-STAT6-1915; SEQ ID NO: 142). In some aspects, the target region corresponds to nucleotides 1916-1932 of SEQ ID NO: 3 (e.g., ASO-STAT6-1916; SEQ ID NO: 143). In some aspects, the target region corresponds to nucleotides 1917-1933 of SEQ ID NO: 3 (e.g., ASO-STAT6-1917; SEQ ID NO: 144). In some aspects, the target region corresponds to nucleotides 1938-1954 of SEQ ID NO: 3 (e.g., ASO-STAT6-1938; SEQ ID NO: 145). In some aspects, the target region corresponds to nucleotides 1939-1955 of SEQ ID NO: 3 (e.g., ASO-STAT6-1939; SEQ ID NO: 146). In some aspects, the target region corresponds to nucleotides 2063-2079 of SEQ ID NO: 3 (e.g., ASO-STAT6-2063; SEQ ID NO: 147). In some aspects, the target region corresponds to nucleotides 2064-2080 of SEQ ID NO: 3 (e.g., ASO-STAT6-2064; SEQ ID NO: 148). In some aspects, the target region corresponds to nucleotides 2065-2081 of SEQ ID NO: 3 (e.g., ASO-STAT6-2065; SEQ ID NO: 149). In some aspects, the target region corresponds to nucleotides 2066-2082 of SEQ ID NO: 3 (e.g., ASO-STAT6-2066; SEQ ID NO: 150). In some aspects, the target region corresponds to nucleotides 2068-2084 of SEQ ID NO: 3 (e.g., ASO-STAT6-2068; SEQ ID NO: 151). In some aspects, the target region corresponds to nucleotides 2187-2203 of SEQ ID NO: 3 (e.g., ASO-STAT6-2187; SEQ ID NO: 152). In some aspects, the target region corresponds to nucleotides 2350-2366 of SEQ ID NO: 3 (e.g., ASO-STAT6-2350; SEQ ID NO: 153). In some aspects, the target region corresponds to nucleotides 2351-2367 of SEQ ID NO: 3 (e.g., ASO-STAT6-2351; SEQ ID NO: 154). In some aspects, the target region corresponds to nucleotides 2352-2368 of SEQ ID NO: 3 (e.g., ASO-STAT6-2352; SEQ ID NO: 155). In some aspects, the target region corresponds to nucleotides 2357-2373 of SEQ ID NO: 3 (e.g., ASO-STAT6-2357; SEQ ID NO: 156). In some aspects, the target region corresponds to nucleotides 513-532 of SEQ ID NO: 3 (e.g., ASO-STAT6-513; SEQ ID NO: 157). In some aspects, the target region corresponds to nucleotides 671-690 of SEQ ID NO: 3 (e.g., ASO-STAT6-671; SEQ ID NO: 158). In some aspects, the target region corresponds to nucleotides 1131-1150 of SEQ ID NO: 3 (e.g., ASO-STAT6-1131; SEQ ID NO: 159). In some aspects, the target region corresponds to nucleotides 1354-1373 of SEQ ID NO: 3 (e.g., ASO-STAT6-1354; SEQ ID NO: 160). In some aspects, the target region corresponds to nucleotides 1355-1374 of SEQ ID NO: 3 (e.g., ASO-STAT6-1355; SEQ ID NO: 161). In some aspects, the target region corresponds to nucleotides 1356-1375 of SEQ ID NO: 3 (e.g., ASO-STAT6-1356; SEQ ID NO: 162). In some aspects, the target region corresponds to nucleotides 1432-1451 of SEQ ID NO: 3 (e.g., ASO-STAT6-1432; SEQ ID NO: 163). In some aspects, the target region corresponds to nucleotides 1555-1574 of SEQ ID NO: 3 (e.g., ASO-STAT6-1555; SEQ ID NO: 164). In some aspects, the target region corresponds to nucleotides 1556-1575 of SEQ ID NO: 3 (e.g., ASO-STAT6-1556; SEQ ID NO: 165). In some aspects, the target region corresponds to nucleotides 1557-1576 of SEQ ID NO: 3 (e.g., ASO-STAT6-1557; SEQ ID NO: 166). In some aspects, the target region corresponds to nucleotides 1558-1577 of SEQ ID NO: 3 (e.g., ASO-STAT6-1558; SEQ ID NO: 167). In some aspects, the target region corresponds to nucleotides 1826-1845 of SEQ ID NO: 3 (e.g., ASO-STAT6-1826; SEQ ID NO: 168). In some aspects, the target region corresponds to nucleotides 1827-1846 of SEQ ID NO: 3 (e.g., ASO-STAT6-1827; SEQ ID NO: 169). In some aspects, the target region corresponds to nucleotides 1833-1852 of SEQ ID NO: 3 (e.g., ASO-STAT6-1833; SEQ ID NO: 170). In some aspects, the target region corresponds to nucleotides 1843-1862 of SEQ ID NO: 3 (e.g., ASO-STAT6-1843; SEQ ID NO: 171). In some aspects, the target region corresponds to nucleotides 1846-1865 of SEQ ID NO: 3 (e.g., ASO-STAT6-1846; SEQ ID NO: 172). In some aspects, the target region corresponds to nucleotides 1847-1866 of SEQ ID NO: 3 (e.g., ASO-STAT6-1847; SEQ ID NO: 173). In some aspects, the target region corresponds to nucleotides 1883-1902 of SEQ ID NO: 3 (e.g., ASO-STAT6-1883; SEQ ID NO: 174). In some aspects, the target region corresponds to nucleotides 1889-1908 of SEQ ID NO: 3 (e.g., ASO-STAT6-1889; SEQ ID NO: 175). In some aspects, the target region corresponds to nucleotides 1890-1909 of SEQ ID NO: 3 (e.g., ASO-STAT6-1890; SEQ ID NO: 176). In some aspects, the target region corresponds to nucleotides 1891-1910 of SEQ ID NO: 3 (e.g., ASO-STAT6-1891; SEQ ID NO: 177). In some aspects, the target region corresponds to nucleotides 1916-1935 of SEQ ID NO: 3 (e.g., ASO-STAT6-1916; SEQ ID NO: 178). In some aspects, the target region corresponds to nucleotides 1917-1936 of SEQ ID NO: 3 (e.g., ASO-STAT6-1917; SEQ ID NO: 179). In some aspects, the target region corresponds to nucleotides 2056-2075 of SEQ ID NO: 3 (e.g., ASO-STAT6-2056; SEQ ID NO: 180). In some aspects, the target region corresponds to nucleotides 2057-2076 of SEQ ID NO: 3 (e.g., ASO-STAT6-2057; SEQ ID NO: 181). In some aspects, the target region corresponds to nucleotides 2060-2079 of SEQ ID NO: 3 (e.g., ASO-STAT6-2060; SEQ ID NO: 182). In some aspects, the target region corresponds to nucleotides 2062-2081 of SEQ ID NO: 3 (e.g., ASO-STAT6-2062; SEQ ID NO: 183). In some aspects, the target region corresponds to nucleotides 2063-2082 of SEQ ID NO: 3 (e.g., ASO-STAT6-2063; SEQ ID NO: 184). In some aspects, the target region corresponds to nucleotides 2065-2084 of SEQ ID NO: 3 (e.g., ASO-STAT6-2065; SEQ ID NO: 185). In some aspects, the target region corresponds to nucleotides 2068-2087 of SEQ ID NO: 3 (e.g., ASO-STAT6-2068; SEQ ID NO: 186). In some aspects, the target region corresponds to nucleotides 2347-2366 of SEQ ID NO: 3 (e.g., ASO-STAT6-2347; SEQ ID NO: 187). In some aspects, the target region corresponds to nucleotides 2348-2367 of SEQ ID NO: 3 (e.g., ASO-STAT6-2348; SEQ ID NO: 188). In some aspects, the target region corresponds to nucleotides 2358-2377 of SEQ ID NO: 3 (e.g., ASO-STAT6-2358; SEQ ID NO: 189). In some aspects, the target region corresponds to nucleotides 2782-2801 of SEQ ID NO: 3 (e.g., ASO-STAT6-2782; SEQ ID NO: 190). In some aspects, the target region corresponds to nucleotides 3070-3089 of SEQ ID NO: 3 (e.g., ASO-STAT6-3070; SEQ ID NO: 191). In some aspects, the target region corresponds to nucleotides 3071-3090 of SEQ ID NO: 3 (e.g., ASO-STAT6-3071; SEQ ID NO: 192). In some aspects, the target region corresponds to nucleotides 3431-3450 of SEQ ID NO: 3 (e.g., ASO-STAT6-3431; SEQ ID NO: 193).

In some aspects, the target region corresponds to nucleotides 1053-1067 of SEQ ID NO: 3 (e.g., ASO-STAT6-1053; SEQ ID NO: 91)±10, ±20, ±30, ±40, ±50, ±60, ±70, ±80, or ±90 nucleotides at the 3′ end and/or the 5′ end. In some aspects, the target region corresponds to nucleotides 1359-1373 of SEQ ID NO: 3 (e.g., ASO-STAT6-1359; SEQ ID NO: 92)±10, ±20, +30, ±40, ±50, ±60, ±70, ±80, or ±90 nucleotides at the 3′ end and/or the 5′ end. In some aspects, the target region corresponds to nucleotides 1890-1904 of SEQ ID NO: 3 (e.g., ASO-STAT6-1890; SEQ ID NO: 93)±10, ±20, ±30, ±40, ±50, ±60, ±70, ±80, or ±90 nucleotides at the 3′ end and/or the 5′ end. In some aspects, the target region corresponds to nucleotides 1892-1906 of SEQ ID NO: 3 (e.g., ASO-STAT6-1892; SEQ ID NO: 94)±10, ±20, ±30, ±40, ±50, ±60, ±70, +80, or ±90 nucleotides at the 3′ end and/or the 5′ end. In some aspects, the target region corresponds to nucleotides 1915-1929 of SEQ ID NO: 3 (e.g., ASO-STAT6-1915; SEQ ID NO: 95)±10, ±20, ±30, ±40, ±50, ±60, ±70, ±80, or ±90 nucleotides at the 3′ end and/or the 5′ end. In some aspects, the target region corresponds to nucleotides 1916-1930 of SEQ ID NO: 3 (e.g., ASO-STAT6-1916; SEQ ID NO: 96)±10, ±20, ±30, ±40, ±50, ±60, ±70, ±80, or ±90 nucleotides at the 3′ end and/or the 5′ end. In some aspects, the target region corresponds to nucleotides 1917-1931 of SEQ ID NO: 3 (e.g., ASO-STAT6-1917; SEQ ID NO: 97)±10, ±20, ±30, ±40, ±50, +60, ±70, ±80, or ±90 nucleotides at the 3′ end and/or the 5′ end. In some aspects, the target region corresponds to nucleotides 1918-1932 of SEQ ID NO: 3 (e.g., ASO-STAT6-1918; SEQ ID NO: 98)±10, ±20, ±30, ±40, ±50, ±60, ±70, ±80, or ±90 nucleotides at the 3′ end and/or the 5′ end. In some aspects, the target region corresponds to nucleotides 1919-1933 of SEQ ID NO: 3 (e.g., ASO-STAT6-1919; SEQ ID NO: 99)±10, ±20, ±30, ±40, ±50, ±60, ±70, ±80, or ±90 nucleotides at the 3′ end and/or the 5′ end. In some aspects, the target region corresponds to nucleotides 1920-1934 of SEQ ID NO: 3 (e.g., ASO-STAT6-1920; SEQ ID NO: 100)±10, ±20, ±30, 40, 50, 60, 70, 80, or ±90 nucleotides at the 3′ end and/or the 5′ end. In some aspects, the target region corresponds to nucleotides 1937-1951 of SEQ ID NO: 3 (e.g., ASO-STAT6-1937; SEQ ID NO: 101)±10, ±20, ±30, ±40, ±50, ±60, ±70, ±80, or ±90 nucleotides at the 3′ end and/or the 5′ end. In some aspects, the target region corresponds to nucleotides 1938-1952 of SEQ ID NO: 3 (e.g., ASO-STAT6-1938; SEQ ID NO: 102)±10, ±20, ±30, ±40, ±50, ±60, ±70, ±80, or ±90 nucleotides at the 3′ end and/or the 5′ end. In some aspects, the target region corresponds to nucleotides 2061-2075 of SEQ ID NO: 3 (e.g., ASO-STAT6-2061; SEQ ID NO: 103)±10, ±20, ±30, ±40, ±50, ±60, ±70, ±80, or ±90 nucleotides at the 3′ end and/or the 5′ end. In some aspects, the target region corresponds to nucleotides 2062-2076 of SEQ ID NO: 3 (e.g., ASO-STAT6-2062; SEQ ID NO: 104)±10, ±20, ±30, ±40, ±50, ±60, ±70, ±80, or ±90 nucleotides at the 3′ end and/or the 5′ end. In some aspects, the target region corresponds to nucleotides 2063-2077 of SEQ ID NO: 3 (e.g., ASO-STAT6-2063; SEQ ID NO: 105)±10, ±20, ±30, ±40, ±50, ±60, ±70, ±80, or ±90 nucleotides at the 3′ end and/or the 5′ end. In some aspects, the target region corresponds to nucleotides 2064-2078 of SEQ ID NO: 3 (e.g., ASO-STAT6-2064; SEQ ID NO: 106)±10, ±20, ±30, ±40, ±50, ±60, ±70, ±80, or ±90 nucleotides at the 3′ end and/or the 5′ end. In some aspects, the target region corresponds to nucleotides 2066-2080 of SEQ ID NO: 3 (e.g., ASO-STAT6-2066; SEQ ID NO: 107)±10, ±20, ±30, ±40, ±50, ±60, ±70, ±80, or ±90 nucleotides at the 3′ end and/or the 5′ end. In some aspects, the target region corresponds to nucleotides 2067-2081 of SEQ ID NO: 3 (e.g., ASO-STAT6-2067; SEQ ID NO: 108)±10, ±20, ±30, ±40, ±50, ±60, ±70, ±80, or ±90 nucleotides at the 3′ end and/or the 5′ end. In some aspects, the target region corresponds to nucleotides 2068-2082 of SEQ ID NO: 3 (e.g., ASO-STAT6-2068; SEQ ID NO: 109)±10, ±20, ±30, ±40, ±50, ±60, ±70, ±80, or ±90 nucleotides at the 3′ end and/or the 5′ end. In some aspects, the target region corresponds to nucleotides 2352-2366 of SEQ ID NO: 3 (e.g., ASO-STAT6-2352; SEQ ID NO: 110)±10, ±20, ±30, ±40, ±50, ±60, ±70, ±80, or ±90 nucleotides at the 3′ end and/or the 5′ end. In some aspects, the target region corresponds to nucleotides 3073-3087 of SEQ ID NO: 3 (e.g., ASO-STAT6-3073; SEQ ID NO: 111)±10, ±20, ±30, ±40, ±50, ±60, ±70, ±80, or ±90 nucleotides at the 3′ end and/or the 5′ end. In some aspects, the target region corresponds to nucleotides 1053-1068 of SEQ ID NO: 3 (e.g., ASO-STAT6-1053; SEQ ID NO: 112)±10, ±20, ±30, ±40, ±50, ±60, ±70, ±80, or ±90 nucleotides at the 3′ end and/or the 5′ end. In some aspects, the target region corresponds to nucleotides 1054-1069 of SEQ ID NO: 3 (e.g., ASO-STAT6-1054; SEQ ID NO: 113)±10, ±20, ±30, ±40, ±50, ±60, ±70, ±80, or ±90 nucleotides at the 3′ end and/or the 5′ end. In some aspects, the target region corresponds to nucleotides 1356-1371 of SEQ ID NO: 3 (e.g., ASO-STAT6-1356; SEQ ID NO: 114)±10, ±20, ±30, ±40, ±50, ±60, ±70, ±80, or ±90 nucleotides at the 3′ end and/or the 5′ end. In some aspects, the target region corresponds to nucleotides 1847-1862 of SEQ ID NO: 3 (e.g., ASO-STAT6-1847; SEQ ID NO: 115)±10, ±20, ±30, ±40, ±50, ±60, ±70, ±80, or ±90 nucleotides at the 3′ end and/or the 5′ end. In some aspects, the target region corresponds to nucleotides 1886-1901 of SEQ ID NO: 3 (e.g., ASO-STAT6-1886; SEQ ID NO: 116)±10, ±20, ±30, ±40, ±50, ±60, ±70, ±80, or ±90 nucleotides at the 3′ end and/or the 5′ end. In some aspects, the target region corresponds to nucleotides 1887-1902 of SEQ ID NO: 3 (e.g., ASO-STAT6-1887; SEQ ID NO: 117)±10, ±20, ±30, ±40, ±50, ±60, ±70, ±80, or ±90 nucleotides at the 3′ end and/or the 5′ end. In some aspects, the target region corresponds to nucleotides 1888-1903 of SEQ ID NO: 3 (e.g., ASO-STAT6-1888; SEQ ID NO: 118)±10, ±20, ±30, ±40, ±50, ±60, ±70, ±80, or ±90 nucleotides at the 3′ end and/or the 5′ end. In some aspects, the target region corresponds to nucleotides 1889-1904 of SEQ ID NO: 3 (e.g., ASO-STAT6-1889; SEQ ID NO: 119)±10, ±20, ±30, ±40, ±50, ±60, ±70, ±80, or ±90 nucleotides at the 3′ end and/or the 5′ end. In some aspects, the target region corresponds to nucleotides 1890-1905 of SEQ ID NO: 3 (e.g., ASO-STAT6-1890; SEQ ID NO: 120)±10, ±20, ±30, ±40, ±50, ±60, ±70, ±80, or ±90 nucleotides at the 3′ end and/or the 5′ end. In some aspects, the target region corresponds to nucleotides 1893-1908 of SEQ ID NO: 3 (e.g., ASO-STAT6-1893; SEQ ID NO: 121)±10, ±20, ±30, ±40, ±50, ±60, ±70, ±80, or ±90 nucleotides at the 3′ end and/or the 5′ end. In some aspects, the target region corresponds to nucleotides 1917-1932 of SEQ ID NO: 3 (e.g., ASO-STAT6-1917; SEQ ID NO: 122)±10, ±20, ±30, ±40, ±50, ±60, ±70, ±80, or ±90 nucleotides at the 3′ end and/or the 5′ end. In some aspects, the target region corresponds to nucleotides 1919-1934 of SEQ ID NO: 3 (e.g., ASO-STAT6-1919; SEQ ID NO: 123)±10, ±20, ±30, ±40, ±50, ±60, ±70, ±80, or ±90 nucleotides at the 3′ end and/or the 5′ end. In some aspects, the target region corresponds to nucleotides 2056-2071 of SEQ ID NO: 3 (e.g., ASO-STAT6-2056; SEQ ID NO: 124)±10, ±20, ±30, ±40, ±50, ±60, ±70, ±80, or ±90 nucleotides at the 3′ end and/or the 5′ end. In some aspects, the target region corresponds to nucleotides 2060-2075 of SEQ ID NO: 3 (e.g., ASO-STAT6-2060; SEQ ID NO: 125)±10, ±20, ±30, ±40, ±50, ±60, ±70, ±80, or ±90 nucleotides at the 3′ end and/or the 5′ end. In some aspects, the target region corresponds to nucleotides 2066-2081 of SEQ ID NO: 3 (e.g., ASO-STAT6-2066; SEQ ID NO: 126)±10, ±20, ±30, ±40, ±50, ±60, ±70, ±80, or ±90 nucleotides at the 3′ end and/or the 5′ end. In some aspects, the target region corresponds to nucleotides 2070-2085 of SEQ ID NO: 3 (e.g., ASO-STAT6-2070; SEQ ID NO: 127)±10, ±20, ±30, ±40, ±50, 60, ±70, ±80, or ±90 nucleotides at the 3′ end and/or the 5′ end. In some aspects, the target region corresponds to nucleotides 2351-2366 of SEQ ID NO: 3 (e.g., ASO-STAT6-2351; SEQ ID NO: 128)±10, ±20, ±30, ±40, ±50, ±60, ±70, ±80, or ±90 nucleotides at the 3′ end and/or the 5′ end. In some aspects, the target region corresponds to nucleotides 2352-2367 of SEQ ID NO: 3 (e.g., ASO-STAT6-2352; SEQ ID NO: 129)±10, ±20, ±30, ±40, ±50, ±60, ±70, ±80, or ±90 nucleotides at the 3′ end and/or the 5′ end. In some aspects, the target region corresponds to nucleotides 2359-2374 of SEQ ID NO: 3 (e.g., ASO-STAT6-2359; SEQ ID NO: 130)±10, ±20, ±30, ±40, ±50, ±60, ±70, ±80, or ±90 nucleotides at the 3′ end and/or the 5′ end. In some aspects, the target region corresponds to nucleotides 3633-3648 of SEQ ID NO: 3 (e.g., ASO-STAT6-3633; SEQ ID NO: 131)±10, ±20, ±30, ±40, ±50, ±60, ±70, ±80, or ±90 nucleotides at the 3′ end and/or the 5′ end. In some aspects, the target region corresponds to nucleotides 673-689 of SEQ ID NO: 3 (e.g., ASO-STAT6-673; SEQ ID NO: 132)±10, ±20, ±30, ±40, ±50, +60, ±70, ±80, or ±90 nucleotides at the 3′ end and/or the 5′ end. In some aspects, the target region corresponds to nucleotides 1052-1068 of SEQ ID NO: 3 (e.g., ASO-STAT6-1052; SEQ ID NO: 133)±10, ±20, ±30, ±40, ±50, ±60, ±70, ±80, or ±90 nucleotides at the 3′ end and/or the 5′ end. In some aspects, the target region corresponds to nucleotides 1356-1372 of SEQ ID NO: 3 (e.g., ASO-STAT6-1356; SEQ ID NO: 134)±10, ±20, ±30, ±40, ±50, ±60, ±70, ±80, or ±90 nucleotides at the 3′ end and/or the 5′ end. In some aspects, the target region corresponds to nucleotides 1357-1373 of SEQ ID NO: 3 (e.g., ASO-STAT6-1357; SEQ ID NO: 135)±10, ±20, ±30, ±40, ±50, ±60, ±70, ±80, or ±90 nucleotides at the 3′ end and/or the 5′ end. In some aspects, the target region corresponds to nucleotides 1359-1375 of SEQ ID NO: 3 (e.g., ASO-STAT6-1359; SEQ ID NO: 136)±10, ±20, ±30, ±40, ±50, ±60, ±70, ±80, or ±90 nucleotides at the 3′ end and/or the 5′ end. In some aspects, the target region corresponds to nucleotides 1360-1376 of SEQ ID NO: 3 (e.g., ASO-STAT6-1360; SEQ ID NO: 137)±10, ±20, ±30, ±40, ±50, ±60, ±70, ±80, or ±90 nucleotides at the 3′ end and/or the 5′ end. In some aspects, the target region corresponds to nucleotides 1839-1855 of SEQ ID NO: 3 (e.g., ASO-STAT6-1839; SEQ ID NO: 138)±10, ±20, ±30, ±40, ±50, ±60, ±70, ±80, or ±90 nucleotides at the 3′ end and/or the 5′ end. In some aspects, the target region corresponds to nucleotides 1848-1864 of SEQ ID NO: 3 (e.g., ASO-STAT6-1848; SEQ ID NO: 139)±10, ±20, ±30, ±40, ±50, ±60, ±70, ±80, or ±90 nucleotides at the 3′ end and/or the 5′ end. In some aspects, the target region corresponds to nucleotides 1849-1865 of SEQ ID NO: 3 (e.g., ASO-STAT6-1849; SEQ ID NO: 140)±10, ±20, ±30, 40, 50, 60, 70, 80, or ±90 nucleotides at the 3′ end and/or the 5′ end. In some aspects, the target region corresponds to nucleotides 1891-1907 of SEQ ID NO: 3 (e.g., ASO-STAT6-1891; SEQ ID NO: 141)±10, ±20, ±30, ±40, ±50, ±60, ±70, ±80, or 90 nucleotides at the 3′ end and/or the 5′ end. In some aspects, the target region corresponds to nucleotides 1915-1931 of SEQ ID NO: 3 (e.g., ASO-STAT6-1915; SEQ ID NO: 142)±10, ±20, ±30, ±40, ±50, ±60, ±70, ±80, or ±90 nucleotides at the 3′ end and/or the 5′ end. In some aspects, the target region corresponds to nucleotides 1916-1932 of SEQ ID NO: 3 (e.g., ASO-STAT6-1916; SEQ ID NO: 143)±10, ±20, ±30, ±40, ±50, ±60, ±70, ±80, or ±90 nucleotides at the 3′ end and/or the 5′ end. In some aspects, the target region corresponds to nucleotides 1917-1933 of SEQ ID NO: 3 (e.g., ASO-STAT6-1917; SEQ ID NO: 144)±10, ±20, ±30, ±40, ±50, ±60, ±70, ±80, or ±90 nucleotides at the 3′ end and/or the 5′ end. In some aspects, the target region corresponds to nucleotides 1938-1954 of SEQ ID NO: 3 (e.g., ASO-STAT6-1938; SEQ ID NO: 145)±10, ±20, ±30, ±40, ±50, ±60, ±70, ±80, or ±90 nucleotides at the 3′ end and/or the 5′ end. In some aspects, the target region corresponds to nucleotides 1939-1955 of SEQ ID NO: 3 (e.g., ASO-STAT6-1939; SEQ ID NO: 146)±10, ±20, ±30, ±40, ±50, ±60, ±70, ±80, or ±90 nucleotides at the 3′ end and/or the 5′ end. In some aspects, the target region corresponds to nucleotides 2063-2079 of SEQ ID NO: 3 (e.g., ASO-STAT6-2063; SEQ ID NO: 147)±10, ±20, ±30, ±40, ±50, ±60, ±70, ±80, or ±90 nucleotides at the 3′ end and/or the 5′ end. In some aspects, the target region corresponds to nucleotides 2064-2080 of SEQ ID NO: 3 (e.g., ASO-STAT6-2064; SEQ ID NO: 148)±10, ±20, ±30, ±40, ±50, ±60, ±70, ±80, or ±90 nucleotides at the 3′ end and/or the 5′ end. In some aspects, the target region corresponds to nucleotides 2065-2081 of SEQ ID NO: 3 (e.g., ASO-STAT6-2065; SEQ ID NO: 149)±10, ±20, ±30, ±40, ±50, ±60, ±70, ±80, or ±90 nucleotides at the 3′ end and/or the 5′ end. In some aspects, the target region corresponds to nucleotides 2066-2082 of SEQ ID NO: 3 (e.g., ASO-STAT6-2066; SEQ ID NO: 150)±10, ±20, ±30, ±40, ±50, ±60, ±70, ±80, or ±90 nucleotides at the 3′ end and/or the 5′ end. In some aspects, the target region corresponds to nucleotides 2068-2084 of SEQ ID NO: 3 (e.g., ASO-STAT6-2068; SEQ ID NO: 151)±10, ±20, ±30, ±40, ±50, ±60, ±70, ±80, or ±90 nucleotides at the 3′ end and/or the 5′ end. In some aspects, the target region corresponds to nucleotides 2187-2203 of SEQ ID NO: 3 (e.g., ASO-STAT6-2187; SEQ ID NO: 152)±10, ±20, ±30, ±40, ±50, ±60, ±70, ±80, or ±90 nucleotides at the 3′ end and/or the 5′ end. In some aspects, the target region corresponds to nucleotides 2350-2366 of SEQ ID NO: 3 (e.g., ASO-STAT6-2350; SEQ ID NO: 153)±10, ±20, ±30, ±40, ±50, ±60, ±70, ±80, or ±90 nucleotides at the 3′ end and/or the 5′ end. In some aspects, the target region corresponds to nucleotides 2351-2367 of SEQ ID NO: 3 (e.g., ASO-STAT6-2351; SEQ ID NO: 154)±10, ±20, ±30, ±40, ±50, ±60, ±70, ±80, or ±90 nucleotides at the 3′ end and/or the 5′ end. In some aspects, the target region corresponds to nucleotides 2352-2368 of SEQ ID NO: 3 (e.g., ASO-STAT6-2352; SEQ ID NO: 155)±10, ±20, ±30, ±40, ±50, ±60, ±70, ±80, or ±90 nucleotides at the 3′ end and/or the 5′ end. In some aspects, the target region corresponds to nucleotides 2357-2373 of SEQ ID NO: 3 (e.g., ASO-STAT6-2357; SEQ ID NO: 156)±10, ±20, ±30, ±40, ±50, ±60, ±70, ±80, or ±90 nucleotides at the 3′ end and/or the 5′ end. In some aspects, the target region corresponds to nucleotides 513-532 of SEQ ID NO: 3 (e.g., ASO-STAT6-513; SEQ ID NO: 157)±10, ±20, ±30, ±40, ±50, +60, ±70, ±80, or ±90 nucleotides at the 3′ end and/or the 5′ end. In some aspects, the target region corresponds to nucleotides 671-690 of SEQ ID NO: 3 (e.g., ASO-STAT6-671; SEQ ID NO: 158)±10, ±20, ±30, ±40, ±50, ±60, ±70, ±80, or ±90 nucleotides at the 3′ end and/or the 5′ end. In some aspects, the target region corresponds to nucleotides 1131-1150 of SEQ ID NO: 3 (e.g., ASO-STAT6-1131; SEQ ID NO: 159)±10, ±20, ±30, ±40, ±50, ±60, ±70, ±80, or ±90 nucleotides at the 3′ end and/or the 5′ end. In some aspects, the target region corresponds to nucleotides 1354-1373 of SEQ ID NO: 3 (e.g., ASO-STAT6-1354; SEQ ID NO: 160)±10, ±20, ±30, ±40, ±50, ±60, ±70, ±80, or ±90 nucleotides at the 3′ end and/or the 5′ end. In some aspects, the target region corresponds to nucleotides 1355-1374 of SEQ ID NO: 3 (e.g., ASO-STAT6-1355; SEQ ID NO: 161)±10, ±20, ±30, ±40, ±50, ±60, ±70, ±80, or ±90 nucleotides at the 3′ end and/or the 5′ end. In some aspects, the target region corresponds to nucleotides 1356-1375 of SEQ ID NO: 3 (e.g., ASO-STAT6-1356; SEQ ID NO: 162)±10, ±20, ±30, ±40, ±50, ±60, ±70, ±80, or ±90 nucleotides at the 3′ end and/or the 5′ end. In some aspects, the target region corresponds to nucleotides 1432-1451 of SEQ ID NO: 3 (e.g., ASO-STAT6-1432; SEQ ID NO: 163)±10, ±20, ±30, ±40, ±50, ±60, ±70, ±80, or ±90 nucleotides at the 3′ end and/or the 5′ end. In some aspects, the target region corresponds to nucleotides 1555-1574 of SEQ ID NO: 3 (e.g., ASO-STAT6-1555; SEQ ID NO: 164)±10, ±20, ±30, ±40, ±50, ±60, ±70, ±80, or ±90 nucleotides at the 3′ end and/or the 5′ end. In some aspects, the target region corresponds to nucleotides 1556-1575 of SEQ ID NO: 3 (e.g., ASO-STAT6-1556; SEQ ID NO: 165)±10, ±20, ±30, ±40, ±50, ±60, ±70, ±80, or ±90 nucleotides at the 3′ end and/or the 5′ end. In some aspects, the target region corresponds to nucleotides 1557-1576 of SEQ ID NO: 3 (e.g., ASO-STAT6-1557; SEQ ID NO: 166)±10, ±20, ±30, ±40, ±50, ±60, ±70, ±80, or ±90 nucleotides at the 3′ end and/or the 5′ end. In some aspects, the target region corresponds to nucleotides 1558-1577 of SEQ ID NO: 3 (e.g., ASO-STAT6-1558; SEQ ID NO: 167)±10, ±20, ±30, ±40, ±50, ±60, ±70, ±80, or ±90 nucleotides at the 3′ end and/or the 5′ end. In some aspects, the target region corresponds to nucleotides 1826-1845 of SEQ ID NO: 3 (e.g., ASO-STAT6-1826; SEQ ID NO: 168)±10, ±20, ±30, ±40, ±50, ±60, ±70, ±80, or ±90 nucleotides at the 3′ end and/or the 5′ end. In some aspects, the target region corresponds to nucleotides 1827-1846 of SEQ ID NO: 3 (e.g., ASO-STAT6-1827; SEQ ID NO: 169)±10, ±20, ±30, ±40, ±50, ±60, ±70, ±80, or ±90 nucleotides at the 3′ end and/or the 5′ end. In some aspects, the target region corresponds to nucleotides 1833-1852 of SEQ ID NO: 3 (e.g., ASO-STAT6-1833; SEQ ID NO: 170)±10, ±20, ±30, ±40, ±50, ±60, ±70, ±80, or ±90 nucleotides at the 3′ end and/or the 5′ end. In some aspects, the target region corresponds to nucleotides 1843-1862 of SEQ ID NO: 3 (e.g., ASO-STAT6-1843; SEQ ID NO: 171)±10, ±20, ±30, ±40, ±50, ±60, ±70, ±80, or ±90 nucleotides at the 3′ end and/or the 5′ end. In some aspects, the target region corresponds to nucleotides 1846-1865 of SEQ ID NO: 3 (e.g., ASO-STAT6-1846; SEQ ID NO: 172)±10, ±20, ±30, ±40, ±50, ±60, ±70, ±80, or ±90 nucleotides at the 3′ end and/or the 5′ end. In some aspects, the target region corresponds to nucleotides 1847-1866 of SEQ ID NO: 3 (e.g., ASO-STAT6-1847; SEQ ID NO: 173)±10, ±20, ±30, ±40, ±50, ±60, ±70, ±80, or ±90 nucleotides at the 3′ end and/or the 5′ end. In some aspects, the target region corresponds to nucleotides 1883-1902 of SEQ ID NO: 3 (e.g., ASO-STAT6-1883; SEQ ID NO: 174)±10, ±20, ±30, ±40, ±50, ±60, ±70, ±80, or ±90 nucleotides at the 3′ end and/or the 5′ end. In some aspects, the target region corresponds to nucleotides 1889-1908 of SEQ ID NO: 3 (e.g., ASO-STAT6-1889; SEQ ID NO: 175)±10, ±20, ±30, ±40, ±50, ±60, ±70, ±80, or ±90 nucleotides at the 3′ end and/or the 5′ end. In some aspects, the target region corresponds to nucleotides 1890-1909 of SEQ ID NO: 3 (e.g., ASO-STAT6-1890; SEQ ID NO: 176)±10, ±20, ±30, ±40, ±50, ±60, ±70, ±80, or ±90 nucleotides at the 3′ end and/or the 5′ end. In some aspects, the target region corresponds to nucleotides 1891-1910 of SEQ ID NO: 3 (e.g., ASO-STAT6-1891; SEQ ID NO: 177)±10, ±20, ±30, ±40, ±50, ±60, ±70, ±80, or ±90 nucleotides at the 3′ end and/or the 5′ end. In some aspects, the target region corresponds to nucleotides 1916-1935 of SEQ ID NO: 3 (e.g., ASO-STAT6-1916; SEQ ID NO: 178)±10, ±20, ±30, ±40, ±50, ±60, ±70, ±80, or ±90 nucleotides at the 3′ end and/or the 5′ end. In some aspects, the target region corresponds to nucleotides 1917-1936 of SEQ ID NO: 3 (e.g., ASO-STAT6-1917; SEQ ID NO: 179)±10, ±20, ±30, ±40, ±50, ±60, ±70, ±80, or ±90 nucleotides at the 3′ end and/or the 5′ end. In some aspects, the target region corresponds to nucleotides 2056-2075 of SEQ ID NO: 3 (e.g., ASO-STAT6-2056; SEQ ID NO: 180)±10, ±20, ±30, ±40, ±50, ±60, ±70, ±80, or ±90 nucleotides at the 3′ end and/or the 5′ end. In some aspects, the target region corresponds to nucleotides 2057-2076 of SEQ ID NO: 3 (e.g., ASO-STAT6-2057; SEQ ID NO: 181)±10, ±20, ±30, ±40, ±50, ±60, ±70, ±80, or ±90 nucleotides at the 3′ end and/or the 5′ end. In some aspects, the target region corresponds to nucleotides 2060-2079 of SEQ ID NO: 3 (e.g., ASO-STAT6-2060; SEQ ID NO: 182)±10, ±20, ±30, ±40, ±50, ±60, ±70, ±80, or ±90 nucleotides at the 3′ end and/or the 5′ end. In some aspects, the target region corresponds to nucleotides 2062-2081 of SEQ ID NO: 3 (e.g., ASO-STAT6-2062; SEQ ID NO: 183)±10, ±20, ±30, ±40, ±50, ±60, ±70, ±80, or ±90 nucleotides at the 3′ end and/or the 5′ end. In some aspects, the target region corresponds to nucleotides 2063-2082 of SEQ ID NO: 3 (e.g., ASO-STAT6-2063; SEQ ID NO: 184)±10, ±20, ±30, ±40, ±50, ±60, ±70, ±80, or ±90 nucleotides at the 3′ end and/or the 5′ end. In some aspects, the target region corresponds to nucleotides 2065-2084 of SEQ ID NO: 3 (e.g., ASO-STAT6-2065; SEQ ID NO: 185)±10, ±20, ±30, ±40, ±50, ±60, ±70, ±80, or ±90 nucleotides at the 3′ end and/or the 5′ end. In some aspects, the target region corresponds to nucleotides 2068-2087 of SEQ ID NO: 3 (e.g., ASO-STAT6-2068; SEQ ID NO: 186)±10, ±20, ±30, ±40, ±50, ±60, ±70, ±80, or ±90 nucleotides at the 3′ end and/or the 5′ end. In some aspects, the target region corresponds to nucleotides 2347-2366 of SEQ ID NO: 3 (e.g., ASO-STAT6-2347; SEQ ID NO: 187)±10, ±20, ±30, ±40, ±50, ±60, ±70, ±80, or ±90 nucleotides at the 3′ end and/or the 5′ end. In some aspects, the target region corresponds to nucleotides 2348-2367 of SEQ ID NO: 3 (e.g., ASO-STAT6-2348; SEQ ID NO: 188)±10, ±20, ±30, ±40, ±50, ±60, ±70, ±80, or ±90 nucleotides at the 3′ end and/or the 5′ end. In some aspects, the target region corresponds to nucleotides 2358-2377 of SEQ ID NO: 3 (e.g., ASO-STAT6-2358; SEQ ID NO: 189)±10, ±20, ±30, ±40, ±50, ±60, ±70, ±80, or ±90 nucleotides at the 3′ end and/or the 5′ end. In some aspects, the target region corresponds to nucleotides 2782-2801 of SEQ ID NO: 3 (e.g., ASO-STAT6-2782; SEQ ID NO: 190)±10, ±20, ±30, ±40, ±50, ±60, ±70, ±80, or ±90 nucleotides at the 3′ end and/or the 5′ end. In some aspects, the target region corresponds to nucleotides 3070-3089 of SEQ ID NO: 3 (e.g., ASO-STAT6-3070; SEQ ID NO: 191)±10, ±20, ±30, ±40, ±50, ±60, ±70, ±80, or ±90 nucleotides at the 3′ end and/or the 5′ end. In some aspects, the target region corresponds to nucleotides 3071-3090 of SEQ ID NO: 3 (e.g., ASO-STAT6-3071; SEQ ID NO: 192)±10, ±20, ±30, ±40, ±50, ±60, ±70, ±80, or ±90 nucleotides at the 3′ end and/or the 5′ end. In some aspects, the target region corresponds to nucleotides 3431-3450 of SEQ ID NO: 3 (e.g., ASO-STAT6-3431; SEQ ID NO: 193)±10, ±20, ±30, ±40, ±50, ±60, ±70, ±80, or ±90 nucleotides at the 3′ end and/or the 5′ end).

In some aspects, the ASO is not TGAGCGAATGGACAGGTCTT (SEQ ID NO: 89). In some aspects, the target region corresponds to a contiguous nucleotide sequence of 10 to 30 nucleotides in length that is complementary to a nucleic acid sequence within nucleotides 1-2056 of SEQ ID NO: 3. In some aspects, the target region corresponds to a contiguous nucleotide sequence of 10 to 30 nucleotides in length that is complementary to a nucleic acid sequence within nucleotides 1-2055 of SEQ ID NO: 3. In some aspects, the target region corresponds to a contiguous nucleotide sequence of 10 to 30 nucleotides in length that is complementary to a nucleic acid sequence within nucleotides 1-2054 of SEQ ID NO: 3. In some aspects, the target region corresponds to a contiguous nucleotide sequence of 10 to 30 nucleotides in length that is complementary to a nucleic acid sequence within nucleotides 1-2053 of SEQ ID NO: 3. In some aspects, the target region corresponds to a contiguous nucleotide sequence of 10 to 30 nucleotides in length that is complementary to a nucleic acid sequence within nucleotides 1-2052 of SEQ ID NO: 3. In some aspects, the target region corresponds to a contiguous nucleotide sequence of 10 to 30 nucleotides in length that is complementary to a nucleic acid sequence within nucleotides 1-2051 of SEQ ID NO: 3. In some aspects, the target region corresponds to a contiguous nucleotide sequence of 10 to 30 nucleotides in length that is complementary to a nucleic acid sequence within nucleotides 1-2050 of SEQ ID NO: 3. In some aspects, the target region corresponds to a contiguous nucleotide sequence of 10 to 30 nucleotides in length that is complementary to a nucleic acid sequence within nucleotides 1-2049 of SEQ ID NO: 3. In some aspects, the target region corresponds to a contiguous nucleotide sequence of 10 to 30 nucleotides in length that is complementary to a nucleic acid sequence within nucleotides 1-2048 of SEQ ID NO: 3. In some aspects, the target region corresponds to a contiguous nucleotide sequence of 10 to 30 nucleotides in length that is complementary to a nucleic acid sequence within nucleotides 1-2047 of SEQ ID NO: 3. In some aspects, the target region corresponds to a contiguous nucleotide sequence of 10 to 30 nucleotides in length that is complementary to a nucleic acid sequence within nucleotides 1-2046 of SEQ ID NO: 3. In some aspects, the target region corresponds to a contiguous nucleotide sequence of 10 to 30 nucleotides in length that is complementary to a nucleic acid sequence within nucleotides 1-2045 of SEQ ID NO: 3. In some aspects, the target region corresponds to a contiguous nucleotide sequence of 10 to 30 nucleotides in length that is complementary to a nucleic acid sequence within nucleotides 1-2044 of SEQ ID NO: 3. In some aspects, the target region corresponds to a contiguous nucleotide sequence of 10 to 30 nucleotides in length that is complementary to a nucleic acid sequence within nucleotides 1-2043 of SEQ ID NO: 3. In some aspects, the target region corresponds to a contiguous nucleotide sequence of 10 to 30 nucleotides in length that is complementary to a nucleic acid sequence within nucleotides 1-2042 of SEQ ID NO: 3. In some aspects, the target region corresponds to a contiguous nucleotide sequence of 10 to 30 nucleotides in length that is complementary to a nucleic acid sequence within nucleotides 1-2041 of SEQ ID NO: 3. In some aspects, the target region corresponds to a contiguous nucleotide sequence of 10 to 30 nucleotides in length that is complementary to a nucleic acid sequence within nucleotides 1-2040 of SEQ ID NO: 3. In some aspects, the target region corresponds to a contiguous nucleotide sequence of 10 to 30 nucleotides in length that is complementary to a nucleic acid sequence within nucleotides 1-2039 of SEQ ID NO: 3. In some aspects, the target region corresponds to a contiguous nucleotide sequence of 10 to 30 nucleotides in length that is complementary to a nucleic acid sequence within nucleotides 1-2038 of SEQ ID NO: 3.

In some aspects, the target region corresponds to a contiguous nucleotide sequence of 10 to 30 nucleotides in length that is complementary to a nucleic acid sequence within nucleotides 2041-3963 of SEQ ID NO: 3. In some aspects, the target region corresponds to a contiguous nucleotide sequence of 10 to 30 nucleotides in length that is complementary to a nucleic acid sequence within nucleotides 2042-3963 of SEQ ID NO: 3. In some aspects, the target region corresponds to a contiguous nucleotide sequence of 10 to 30 nucleotides in length that is complementary to a nucleic acid sequence within nucleotides 2043-3963 of SEQ ID NO: 3. In some aspects, the target region corresponds to a contiguous nucleotide sequence of 10 to 30 nucleotides in length that is complementary to a nucleic acid sequence within nucleotides 2044-3963 of SEQ ID NO: 3. In some aspects, the target region corresponds to a contiguous nucleotide sequence of 10 to 30 nucleotides in length that is complementary to a nucleic acid sequence within nucleotides 2045-3963 of SEQ ID NO: 3. In some aspects, the target region corresponds to a contiguous nucleotide sequence of 10 to 30 nucleotides in length that is complementary to a nucleic acid sequence within nucleotides 2046-3963 of SEQ ID NO: 3. In some aspects, the target region corresponds to a contiguous nucleotide sequence of 10 to 30 nucleotides in length that is complementary to a nucleic acid sequence within nucleotides 2047-3963 of SEQ ID NO: 3. In some aspects, the target region corresponds to a contiguous nucleotide sequence of 10 to 30 nucleotides in length that is complementary to a nucleic acid sequence within nucleotides 2048-3963 of SEQ ID NO: 3. In some aspects, the target region corresponds to a contiguous nucleotide sequence of 10 to 30 nucleotides in length that is complementary to a nucleic acid sequence within nucleotides 2049-3963 of SEQ ID NO: 3. In some aspects, the target region corresponds to a contiguous nucleotide sequence of 10 to 30 nucleotides in length that is complementary to a nucleic acid sequence within nucleotides 2050-3963 of SEQ ID NO: 3. In some aspects, the target region corresponds to a contiguous nucleotide sequence of 10 to 30 nucleotides in length that is complementary to a nucleic acid sequence within nucleotides 2051-3963 of SEQ ID NO: 3. In some aspects, the target region corresponds to a contiguous nucleotide sequence of 10 to 30 nucleotides in length that is complementary to a nucleic acid sequence within nucleotides 2052-3963 of SEQ ID NO: 3. In some aspects, the target region corresponds to a contiguous nucleotide sequence of 10 to 30 nucleotides in length that is complementary to a nucleic acid sequence within nucleotides 2053-3963 of SEQ ID NO: 3. In some aspects, the target region corresponds to a contiguous nucleotide sequence of 10 to 30 nucleotides in length that is complementary to a nucleic acid sequence within nucleotides 2054-3963 of SEQ ID NO: 3. In some aspects, the target region corresponds to a contiguous nucleotide sequence of 10 to 30 nucleotides in length that is complementary to a nucleic acid sequence within nucleotides 2055-3963 of SEQ ID NO: 3. In some aspects, the target region corresponds to a contiguous nucleotide sequence of 10 to 30 nucleotides in length that is complementary to a nucleic acid sequence within nucleotides 2056-3963 of SEQ ID NO: 3. In some aspects, the target region corresponds to a contiguous nucleotide sequence of 10 to 30 nucleotides in length that is complementary to a nucleic acid sequence within nucleotides 2057-3963 of SEQ ID NO: 3. In some aspects, the target region corresponds to a contiguous nucleotide sequence of 10 to 30 nucleotides in length that is complementary to a nucleic acid sequence within nucleotides 2058-3963 of SEQ ID NO: 3. In some aspects, the target region corresponds to a contiguous nucleotide sequence of 10 to 30 nucleotides in length that is complementary to a nucleic acid sequence within nucleotides 2059-3963 of SEQ ID NO: 3.

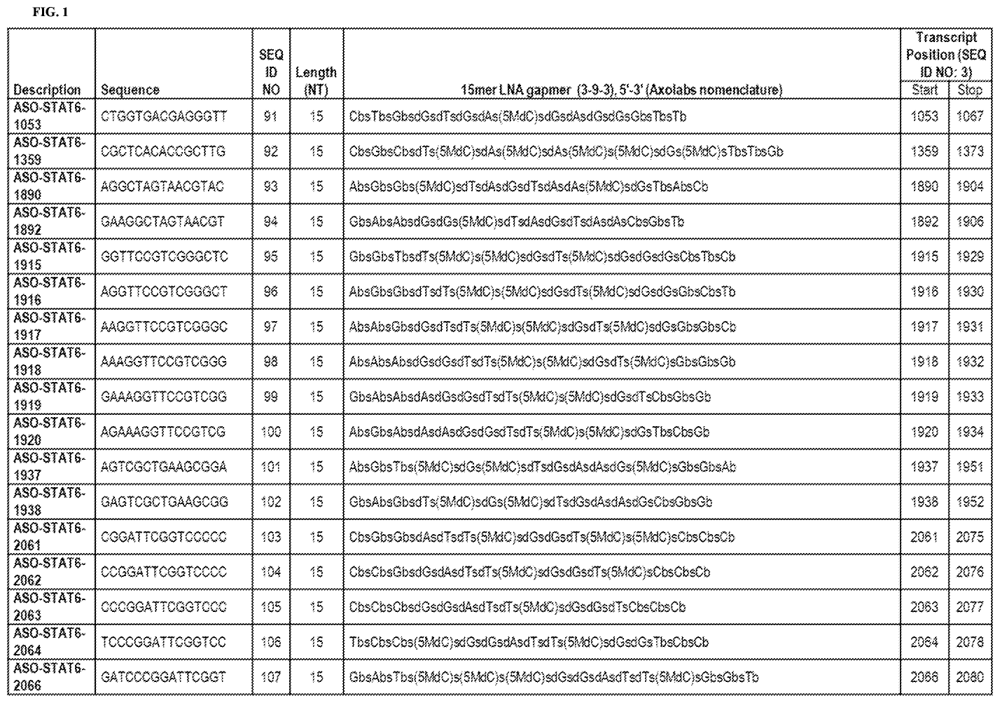

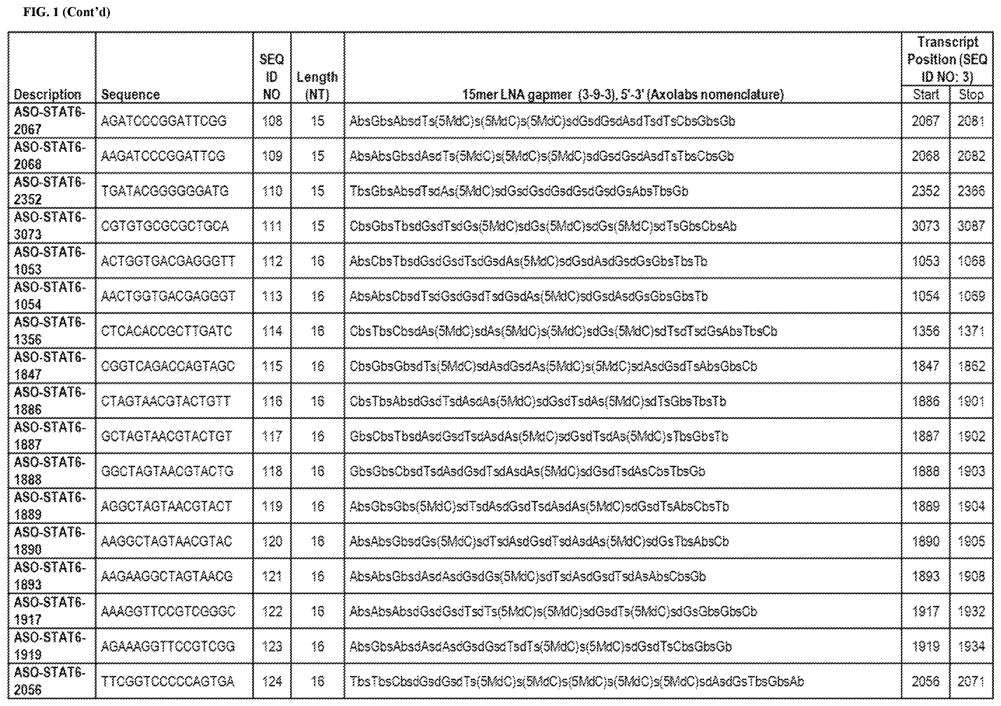

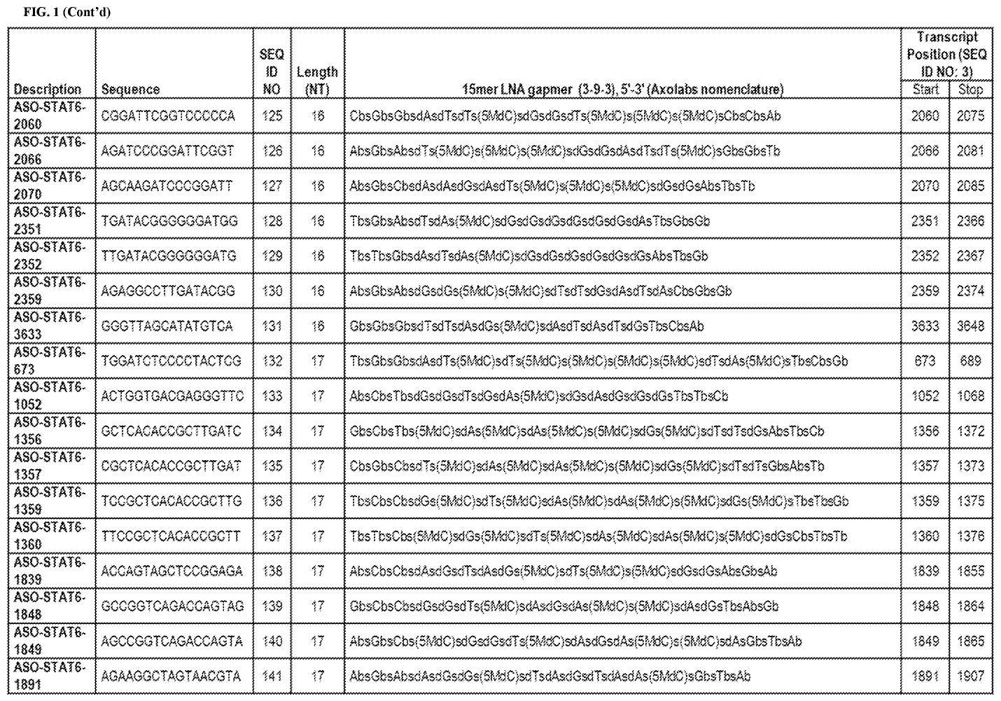

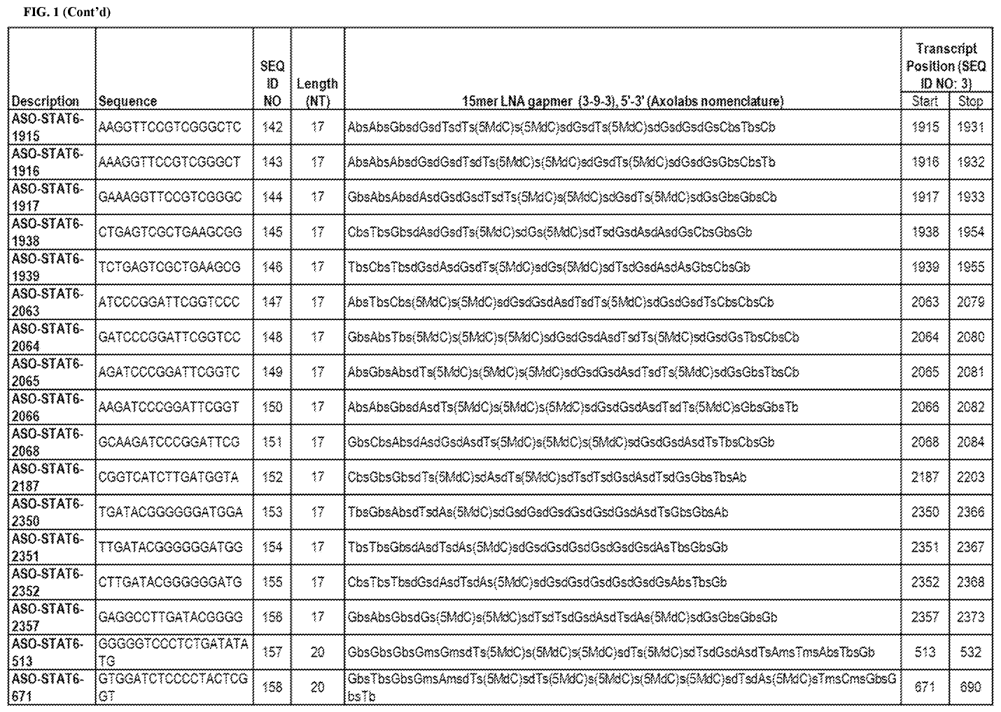

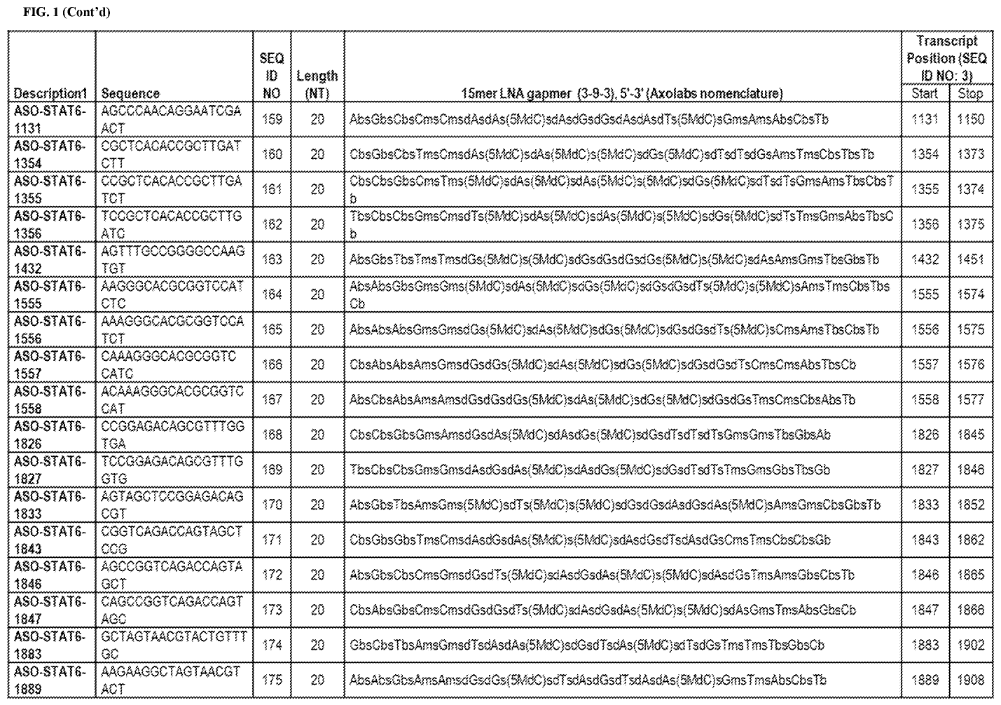

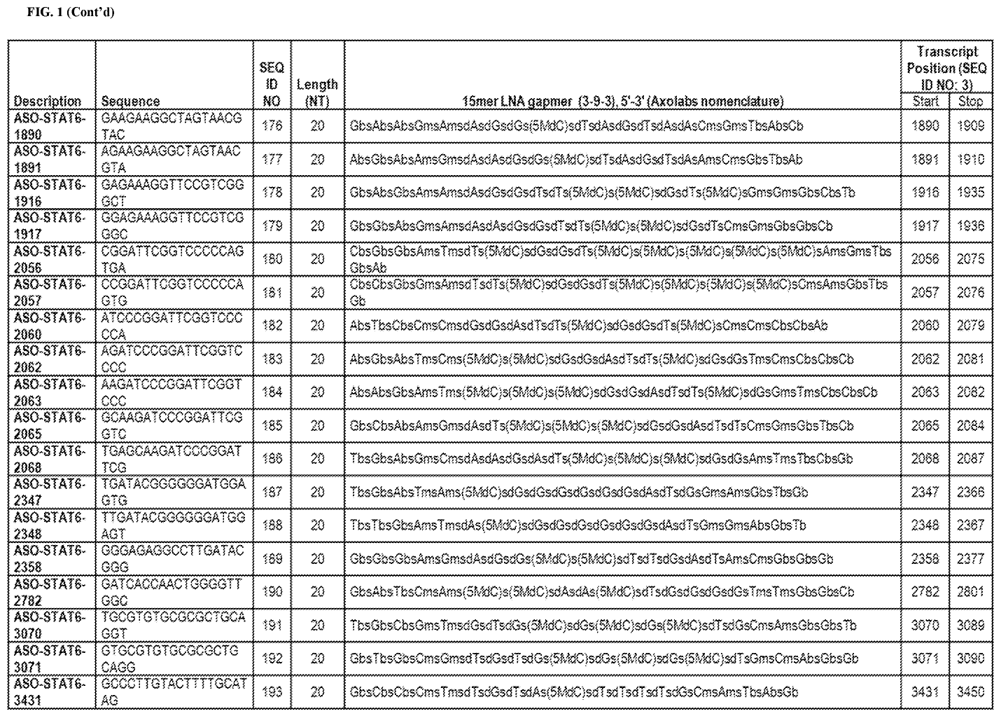

In some aspects, the ASO of the present disclosure hybridizes to multiple target regions within the STAT6 transcript (e.g., genomic sequence, SEQ ID NO: 1). In some aspects, the ASO hybridizes to two different target regions within the STAT6 transcript. In some aspects, the ASO hybridizes to three different target regions within the STAT6 transcript. The sequences of exemplary ASOs that hybridize to multiple target regions, and the start/end sites of the different target regions are provided in FIG. 1A. In some aspects, the ASOs that hybridizes to multiple regions within the STAT6 transcript (e.g., genomic sequence, SEQ ID NO: 1) are more potent (e.g., having lower EC50) at reducing STAT6 expression compared to ASOs that hybridizes to a single region within the STAT6 transcript (e.g., genomic sequence, SEQ ID NO: 1).

### II.A.2. ASO STAT6 Sequences

In some aspects, the ASOs of the disclosure comprise a contiguous nucleotide sequence which corresponds to the complement of a region of STAT6 transcript, e.g., a nucleotide sequence corresponding to SEQ ID NO: 1 or SEQ ID NO: 3.

In certain aspects, the disclosure provides an ASO from 10-30, such as 10-15 nucleotides, 10-20 nucleotides, 10-25 nucleotides in length, or about 20 nucleotides in length, wherein the contiguous nucleotide sequence has at least about 80%, at least about 85%, at least about 90%, at least about 95%, at least about 96%, at least about 97%, at least about 98%, at least about 99%, or about 100% sequence identity to a region within the complement of: a STAT6 transcript, such as SEQ ID NO: 1 or SEQ ID NO: 3 or naturally occurring variant thereof. Thus, for example, the ASO hybridizes to a single stranded nucleic acid molecule having the sequence of SEQ ID NO: 1 or SEQ ID NO: 3 or a portion thereof.

The ASO can comprise a contiguous nucleotide sequence which is fully complementary (perfectly complementary) to the equivalent region of a nucleic acid which encodes a mammalian STAT6 protein (e.g., SEQ ID NO: 1 or SEQ ID NO: 3). The ASO can comprise a contiguous nucleotide sequence which is fully complementary (perfectly complementary) to a nucleic acid sequence, or a region within the sequence, corresponding to nucleotides X-Y of SEQ ID NO: 1 or SEQ ID NO: 3, wherein X and Y are the start site and the end site, respectively, as shown in FIG. 1A.

The ASO can comprise a contiguous nucleotide sequence which is fully complementary (perfectly complementary) to the equivalent region of a mRNA which encodes a mammalian STAT6 protein (e.g., SEQ ID NO: 3). The ASO can comprise a contiguous nucleotide sequence which is fully complementary (perfectly complementary) to a mRNA sequence, or a region within the sequence, corresponding to nucleotides X-Y of SEQ ID NO: 3, wherein X and Y are the start site and the end site, respectively.

In some aspects, the nucleotide sequence of the ASOs of the disclosure or the contiguous nucleotide sequence has at least about 80% sequence identity to a sequence selected from SEQ ID NOs: 91 to 193 (i.e., the sequences in FIG. 1A), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some aspects, the ASO has a design described elsewhere herein or a chemical structure shown elsewhere herein (e.g., FIG. 1A).

In some aspects the ASO (or contiguous nucleotide portion thereof) is selected from, or comprises, one of the sequences selected from the group consisting of SEQ ID NOs: 91 to 193 or a region of at least 10 contiguous nucleotides thereof, wherein the ASO (or contiguous nucleotide portion thereof) can optionally comprise one, two, three, or four mismatches when compared to the corresponding STAT6 transcript.

In some aspects, the ASO comprises a sequence selected from the group consisting of 91 (e.g., ASO-STAT6-1053), 92 (e.g., ASO-STAT6-1359), 93 (e.g., ASO-STAT6-1890), 94 (e.g., ASO-STAT6-1892), 95 (e.g., ASO-STAT6-1915), 96 (e.g., ASO-STAT6-1916), 97 (e.g., ASO-STAT6-1917), 98 (e.g., ASO-STAT6-1918), 99 (e.g., ASO-STAT6-1919), 100 (e.g., ASO-STAT6-1920), 101 (e.g., ASO-STAT6-1937), 102 (e.g., ASO-STAT6-1938), 103 (e.g., ASO-STAT6-2061), 104 (e.g., ASO-STAT6-2062), 105 (e.g., ASO-STAT6-2063), 106 (e.g., ASO-STAT6-2064), 107 (e.g., ASO-STAT6-2066), 108 (e.g., ASO-STAT6-2067), 109 (e.g., ASO-STAT6-2068), 110 (e.g., ASO-STAT6-2352), 111 (e.g., ASO-STAT6-3073), 112 (e.g., ASO-STAT6-1053), 113 (e.g., ASO-STAT6-1054), 114 (e.g., ASO-STAT6-1356), 115 (e.g., ASO-STAT6-1847), 116 (e.g., ASO-STAT6-1886), 117 (e.g., ASO-STAT6-1887), 118 (e.g., ASO-STAT6-1888), 119 (e.g., ASO-STAT6-1889), 120 (e.g., ASO-STAT6-1890), 121 (e.g., ASO-STAT6-1893), 122 (e.g., ASO-STAT6-1917), 123 (e.g., ASO-STAT6-1919), 124 (e.g., ASO-STAT6-2056), 125 (e.g., ASO-STAT6-2060), 126 (e.g., ASO-STAT6-2066), 127 (e.g., ASO-STAT6-2070), 128 (e.g., ASO-STAT6-2351), 129 (e.g., ASO-STAT6-2352), 130 (e.g., ASO-STAT6-2359), 131 (e.g., ASO-STAT6-3633), 132 (e.g., ASO-STAT6-673), 133 (e.g., ASO-STAT6-1052), 134 (e.g., ASO-STAT6-1356), 135 (e.g., ASO-STAT6-1357), 136 (e.g., ASO-STAT6-1359), 137 (e.g., ASO-STAT6-1360), 138 (e.g., ASO-STAT6-1839), 139 (e.g., ASO-STAT6-1848), 140 (e.g., ASO-STAT6-1849), 141 (e.g., ASO-STAT6-1891), 142 (e.g., ASO-STAT6-1915), 143 (e.g., ASO-STAT6-1916), 144 (e.g., ASO-STAT6-1917), 145 (e.g., ASO-STAT6-1938), 146 (e.g., ASO-STAT6-1939), 147 (e.g., ASO-STAT6-2063), 148 (e.g., ASO-STAT6-2064), 149 (e.g., ASO-STAT6-2065), 150 (e.g., ASO-STAT6-2066), 151 (e.g., ASO-STAT6-2068), 152 (e.g., ASO-STAT6-2187), 153 (e.g., ASO-STAT6-2350), 154 (e.g., ASO-STAT6-2351), 155 (e.g., ASO-STAT6-2352), 156 (e.g., ASO-STAT6-2357), 157 (e.g., ASO-STAT6-513), 158 (e.g., ASO-STAT6-671), 159 (e.g., ASO-STAT6-1131), 160 (e.g., ASO-STAT6-1354), 161 (e.g., ASO-STAT6-1355), 162 (e.g., ASO-STAT6-1356), 163 (e.g., ASO-STAT6-1432), 164 (e.g., ASO-STAT6-1555), 165 (e.g., ASO-STAT6-1556), 166 (e.g., ASO-STAT6-1557), 167 (e.g., ASO-STAT6-1558), 168 (e.g., ASO-STAT6-1826), 169 (e.g., ASO-STAT6-1827), 170 (e.g., ASO-STAT6-1833), 171 (e.g., ASO-STAT6-1843), 172 (e.g., ASO-STAT6-1846), 173 (e.g., ASO-STAT6-1847), 174 (e.g., ASO-STAT6-1883), 175 (e.g., ASO-STAT6-1889), 176 (e.g., ASO-STAT6-1890), 177 (e.g., ASO-STAT6-1891), 178 (e.g., ASO-STAT6-1916), 179 (e.g., ASO-STAT6-1917), 180 (e.g., ASO-STAT6-2056), 181 (e.g., ASO-STAT6-2057), 182 (e.g., ASO-STAT6-2060), 183 (e.g., ASO-STAT6-2062), 184 (e.g., ASO-STAT6-2063), 185 (e.g., ASO-STAT6-2065), 186 (e.g., ASO-STAT6-2068), 187 (e.g., ASO-STAT6-2347), 188 (e.g., ASO-STAT6-2348), 189 (e.g., ASO-STAT6-2358), 190 (e.g., ASO-STAT6-2782), 191 (e.g., ASO-STAT6-3070), 192 (e.g., ASO-STAT6-3071), and 193 (e.g., ASO-STAT6-3431).

In some aspects, the ASO comprises the sequence as set forth in SEQ ID NO: 91 (e.g., ASO-STAT6-1053). In some aspects, the ASO comprises the sequence as set forth in SEQ ID NO: 92 (e.g., ASO-STAT6-1359). In some aspects, the ASO comprises the sequence as set forth in SEQ ID NO: 93 (e.g., ASO-STAT6-1890). In some aspects, the ASO comprises the sequence as set forth in SEQ ID NO: 94 (e.g., ASO-STAT6-1892). In some aspects, the ASO comprises the sequence as set forth in SEQ ID NO: 95 (e.g., ASO-STAT6-1915). In some aspects, the ASO comprises the sequence as set forth in SEQ ID NO: 96 (e.g., ASO-STAT6-1916). In some aspects, the ASO comprises the sequence as set forth in SEQ ID NO: 97 (e.g., ASO-STAT6-1917). In some aspects, the ASO comprises the sequence as set forth in SEQ ID NO: 98 (e.g., ASO-STAT6-1918). In some aspects, the ASO comprises the sequence as set forth in SEQ ID NO: 99 (e.g., ASO-STAT6-1919). In some aspects, the ASO comprises the sequence as set forth in SEQ ID NO: 100 (e.g., ASO-STAT6-1920). In some aspects, the ASO comprises the sequence as set forth in SEQ ID NO: 101 (e.g., ASO-STAT6-1937). In some aspects, the ASO comprises the sequence as set forth in SEQ ID NO: 102 (e.g., ASO-STAT6-1938). In some aspects, the ASO comprises the sequence as set forth in SEQ ID NO: 103 (e.g., ASO-STAT6-2061). In some aspects, the ASO comprises the sequence as set forth in SEQ ID NO: 104 (e.g., ASO-STAT6-2062). In some aspects, the ASO comprises the sequence as set forth in SEQ ID NO: 105 (e.g., ASO-STAT6-2063). In some aspects, the ASO comprises the sequence as set forth in SEQ ID NO: 106 (e.g., ASO-STAT6-2064). In some aspects, the ASO comprises the sequence as set forth in SEQ ID NO: 107 (e.g., ASO-STAT6-2066). In some aspects, the ASO comprises the sequence as set forth in SEQ ID NO: 108 (e.g., ASO-STAT6-2067). In some aspects, the ASO comprises the sequence as set forth in SEQ ID NO: 109 (e.g., ASO-STAT6-2068). In some aspects, the ASO comprises the sequence as set forth in SEQ ID NO: 110 (e.g., ASO-STAT6-2352). In some aspects, the ASO comprises the sequence as set forth in SEQ ID NO: 111 (e.g., ASO-STAT6-3073). In some aspects, the ASO comprises the sequence as set forth in SEQ ID NO: 112 (e.g., ASO-STAT6-1053). In some aspects, the ASO comprises the sequence as set forth in SEQ ID NO: 113 (e.g., ASO-STAT6-1054). In some aspects, the ASO comprises the sequence as set forth in SEQ ID NO: 114 (e.g., ASO-STAT6-1356). In some aspects, the ASO comprises the sequence as set forth in SEQ ID NO: 115 (e.g., ASO-STAT6-1847). In some aspects, the ASO comprises the sequence as set forth in SEQ ID NO: 116 (e.g., ASO-STAT6-1886). In some aspects, the ASO comprises the sequence as set forth in SEQ ID NO: 117 (e.g., ASO-STAT6-1887). In some aspects, the ASO comprises the sequence as set forth in SEQ ID NO: 118 (e.g., ASO-STAT6-1888). In some aspects, the ASO comprises the sequence as set forth in SEQ ID NO: 119 (e.g., ASO-STAT6-1889). In some aspects, the ASO comprises the sequence as set forth in SEQ ID NO: 120 (e.g., ASO-STAT6-1890). In some aspects, the ASO comprises the sequence as set forth in SEQ ID NO: 121 (e.g., ASO-STAT6-1893). In some aspects, the ASO comprises the sequence as set forth in SEQ ID NO: 122 (e.g., ASO-STAT6-1917). In some aspects, the ASO comprises the sequence as set forth in SEQ ID NO: 123 (e.g., ASO-STAT6-1919). In some aspects, the ASO comprises the sequence as set forth in SEQ ID NO: 124 (e.g., ASO-STAT6-2056). In some aspects, the ASO comprises the sequence as set forth in SEQ ID NO: 125 (e.g., ASO-STAT6-2060). In some aspects, the ASO comprises the sequence as set forth in SEQ ID NO: 126 (e.g., ASO-STAT6-2066). In some aspects, the ASO comprises the sequence as set forth in SEQ ID NO: 127 (e.g., ASO-STAT6-2070). In some aspects, the ASO comprises the sequence as set forth in SEQ ID NO: 128 (e.g., ASO-STAT6-2351). In some aspects, the ASO comprises the sequence as set forth in SEQ ID NO: 129 (e.g., ASO-STAT6-2352). In some aspects, the ASO comprises the sequence as set forth in SEQ ID NO: 130 (e.g., ASO-STAT6-2359). In some aspects, the ASO comprises the sequence as set forth in SEQ ID NO: 131 (e.g., ASO-STAT6-3633). In some aspects, the ASO comprises the sequence as set forth in SEQ ID NO: 132 (e.g., ASO-STAT6-673). In some aspects, the ASO comprises the sequence as set forth in SEQ ID NO: 133 (e.g., ASO-STAT6-1052). In some aspects, the ASO comprises the sequence as set forth in SEQ ID NO: 134 (e.g., ASO-STAT6-1356). In some aspects, the ASO comprises the sequence as set forth in SEQ ID NO: 135 (e.g., ASO-STAT6-1357). In some aspects, the ASO comprises the sequence as set forth in SEQ ID NO: 136 (e.g., ASO-STAT6-1359). In some aspects, the ASO comprises the sequence as set forth in SEQ ID NO: 137 (e.g., ASO-STAT6-1360). In some aspects, the ASO comprises the sequence as set forth in SEQ ID NO: 138 (e.g., ASO-STAT6-1839). In some aspects, the ASO comprises the sequence as set forth in SEQ ID NO: 139 (e.g., ASO-STAT6-1848). In some aspects, the ASO comprises the sequence as set forth in SEQ ID NO: 140 (e.g., ASO-STAT6-1849). In some aspects, the ASO comprises the sequence as set forth in SEQ ID NO: 141 (e.g., ASO-STAT6-1891). In some aspects, the ASO comprises the sequence as set forth in SEQ ID NO: 142 (e.g., ASO-STAT6-1915). In some aspects, the ASO comprises the sequence as set forth in SEQ ID NO: 143 (e.g., ASO-STAT6-1916). In some aspects, the ASO comprises the sequence as set forth in SEQ ID NO: 144 (e.g., ASO-STAT6-1917). In some aspects, the ASO comprises the sequence as set forth in SEQ ID NO: 145 (e.g., ASO-STAT6-1938). In some aspects, the ASO comprises the sequence as set forth in SEQ ID NO: 146 (e.g., ASO-STAT6-1939). In some aspects, the ASO comprises the sequence as set forth in SEQ ID NO: 147 (e.g., ASO-STAT6-2063). In some aspects, the ASO comprises the sequence as set forth in SEQ ID NO: 148 (e.g., ASO-STAT6-2064). In some aspects, the ASO comprises the sequence as set forth in SEQ ID NO: 149 (e.g., ASO-STAT6-2065). In some aspects, the ASO comprises the sequence as set forth in SEQ ID NO: 150 (e.g., ASO-STAT6-2066). In some aspects, the ASO comprises the sequence as set forth in SEQ ID NO: 151 (e.g., ASO-STAT6-2068). In some aspects, the ASO comprises the sequence as set forth in SEQ ID NO: 152 (e.g., ASO-STAT6-2187). In some aspects, the ASO comprises the sequence as set forth in SEQ ID NO: 153 (e.g., ASO-STAT6-2350). In some aspects, the ASO comprises the sequence as set forth in SEQ ID NO: 154 (e.g., ASO-STAT6-2351). In some aspects, the ASO comprises the sequence as set forth in SEQ ID NO: 155 (e.g., ASO-STAT6-2352). In some aspects, the ASO comprises the sequence as set forth in SEQ ID NO: 156 (e.g., ASO-STAT6-2357). In some aspects, the ASO comprises the sequence as set forth in SEQ ID NO: 157 (e.g., ASO-STAT6-513). In some aspects, the ASO comprises the sequence as set forth in SEQ ID NO: 158 (e.g., ASO-STAT6-671). In some aspects, the ASO comprises the sequence as set forth in SEQ ID NO: 159 (e.g., ASO-STAT6-1131). In some aspects, the ASO comprises the sequence as set forth in SEQ ID NO: 160 (e.g., ASO-STAT6-1354). In some aspects, the ASO comprises the sequence as set forth in SEQ ID NO: 161 (e.g., ASO-STAT6-1355). In some aspects, the ASO comprises the sequence as set forth in SEQ ID NO: 162 (e.g., ASO-STAT6-1356). In some aspects, the ASO comprises the sequence as set forth in SEQ ID NO: 163 (e.g., ASO-STAT6-1432). In some aspects, the ASO comprises the sequence as set forth in SEQ ID NO: 164 (e.g., ASO-STAT6-1555). In some aspects, the ASO comprises the sequence as set forth in SEQ ID NO: 165 (e.g., ASO-STAT6-1556). In some aspects, the ASO comprises the sequence as set forth in SEQ ID NO: 166 (e.g., ASO-STAT6-1557). In some aspects, the ASO comprises the sequence as set forth in SEQ ID NO: 167 (e.g., ASO-STAT6-1558). In some aspects, the ASO comprises the sequence as set forth in SEQ ID NO: 168 (e.g., ASO-STAT6-1826). In some aspects, the ASO comprises the sequence as set forth in SEQ ID NO: 169 (e.g., ASO-STAT6-1827). In some aspects, the ASO comprises the sequence as set forth in SEQ ID NO: 170 (e.g., ASO-STAT6-1833). In some aspects, the ASO comprises the sequence as set forth in SEQ ID NO: 171 (e.g., ASO-STAT6-1843). In some aspects, the ASO comprises the sequence as set forth in SEQ ID NO: 172 (e.g., ASO-STAT6-1846). In some aspects, the ASO comprises the sequence as set forth in SEQ ID NO: 173 (e.g., ASO-STAT6-1847). In some aspects, the ASO comprises the sequence as set forth in SEQ ID NO: 174 (e.g., ASO-STAT6-1883). In some aspects, the ASO comprises the sequence as set forth in SEQ ID NO: 175 (e.g., ASO-STAT6-1889). In some aspects, the ASO comprises the sequence as set forth in SEQ ID NO: 176 (e.g., ASO-STAT6-1890). In some aspects, the ASO comprises the sequence as set forth in SEQ ID NO: 177 (e.g., ASO-STAT6-1891). In some aspects, the ASO comprises the sequence as set forth in SEQ ID NO: 178 (e.g., ASO-STAT6-1916). In some aspects, the ASO comprises the sequence as set forth in SEQ ID NO: 179 (e.g., ASO-STAT6-1917). In some aspects, the ASO comprises the sequence as set forth in SEQ ID NO: 180 (e.g., ASO-STAT6-2056). In some aspects, the ASO comprises the sequence as set forth in SEQ ID NO: 181 (e.g., ASO-STAT6-2057). In some aspects, the ASO comprises the sequence as set forth in SEQ ID NO: 182 (e.g., ASO-STAT6-2060). In some aspects, the ASO comprises the sequence as set forth in SEQ ID NO: 183 (e.g., ASO-STAT6-2062). In some aspects, the ASO comprises the sequence as set forth in SEQ ID NO: 184 (e.g., ASO-STAT6-2063). In some aspects, the ASO comprises the sequence as set forth in SEQ ID NO: 185 (e.g., ASO-STAT6-2065). In some aspects, the ASO comprises the sequence as set forth in SEQ ID NO: 186 (e.g., ASO-STAT6-2068). In some aspects, the ASO comprises the sequence as set forth in SEQ ID NO: 187 (e.g., ASO-STAT6-2347). In some aspects, the ASO comprises the sequence as set forth in SEQ ID NO: 188 (e.g., ASO-STAT6-2348). In some aspects, the ASO comprises the sequence as set forth in SEQ ID NO: 189 (e.g., ASO-STAT6-2358). In some aspects, the ASO comprises the sequence as set forth in SEQ ID NO: 190 (e.g., ASO-STAT6-2782). In some aspects, the ASO comprises the sequence as set forth in SEQ ID NO: 191 (e.g., ASO-STAT6-3070). In some aspects, the ASO comprises the sequence as set forth in SEQ ID NO: 192 (e.g., ASO-STAT6-3071). In some aspects, the ASO comprises the sequence as set forth in SEQ ID NO: 193 (e.g., ASO-STAT6-3431).

In some aspects, the ASOs of the disclosure bind to the target nucleic acid sequence (e.g., STAT6 transcript) and are capable of inhibiting or reducing expression of the STAT6 transcript by at least 10% or 20% compared to the normal (i.e., control) expression level in the cell, e.g., at least about 30%, at least about 40%, at least about 50%, at least about 60%, at least about 70%, at least about 80%, at least about 90%, at least about 95%, at least about 96%, at least about 97%, at least about 98%, at least about 99%, or about 100% compared to the normal expression level (e.g., expression level in cells that have not been exposed to the ASO).

In some aspects, the ASOs of the disclosure are capable of reducing expression of STAT6 mRNA in vitro by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% in target cells when the cells are in contact with the ASO compared to cells that are not in contact with the ASO (e.g., contact with saline).

### II.A.3. ASO Length

The ASOs can comprise a contiguous nucleotide sequence of a total of 10, 11, 12, 13, 14, 15, 16, 17, 18, 19, 20, 21, 22, 23, 24, 25, 26, 27, 28, 29, or 30 contiguous nucleotides in length. It should be understood that when a range is given for an ASO, or contiguous nucleotide sequence length, the range includes the lower and upper lengths provided in the range, for example from (or between) 10-30, includes both 10 and 30.

In some aspects, the ASOs comprise a contiguous nucleotide sequence of a total of about 14-20, 14, 15, 16, 17, 18, 19, or 20 contiguous nucleotides in length. In certain aspects, the ASOs comprise a contiguous nucleotide sequence of a total of about 20 contiguous nucleotides in length. In certain aspects, ASOs of the present disclosure are 14 nucleotides in length. In certain aspects, ASOs of the present disclosure are 15 nucleotides in length. In certain aspects, ASOs of the present disclosure are 16 nucleotides in length. In certain aspects, ASOs of the present disclosure are 17 nucleotides in length. In certain aspects, ASOs of the present disclosure are 18 nucleotides in length. In certain aspects, ASOs of the present disclosure are 19 nucleotides in length.

### II.A.4. Nucleosides and Nucleoside Analogs

In one aspect of the disclosure, the ASOs comprise one or more non-naturally occurring nucleoside analogs. “Nucleoside analogs” as used herein are variants of natural nucleosides, such as DNA or RNA nucleosides, by virtue of modifications in the sugar and/or base moieties. Analogs could in principle be merely “silent” or “equivalent” to the natural nucleosides in the context of the oligonucleotide, i.e. have no functional effect on the way the oligonucleotide works to inhibit target gene expression. Such “equivalent” analogs can nevertheless be useful if, for example, they are easier or cheaper to manufacture, or are more stable to storage or manufacturing conditions, or represent a tag or label. In some aspects, however, the analogs will have a functional effect on the way in which the ASO works to inhibit expression; for example by producing increased binding affinity to the target and/or increased resistance to intracellular nucleases and/or increased ease of transport into the cell. Specific examples of nucleoside analogs are described by e.g. Freier & Altmann; Nucl. Acid Res., 1997, 25, 4429-4443 and Uhlmann; Curr. Opinion in Drug Development, 2000, 3(2), 293-213, and in Scheme 1. The ASOs of the present disclosure can contain more than one, more than two, more than three, more than four, more than five, more than six, more than seven, more than eight, more than nine, more than 10, more than 11, more than 12, more than 13, more than 14, more than 15, more than 16, more than 18, more than 19, or more than 20 nucleoside analogs. In some aspects, the nucleoside analogs in the ASOs are the same. In other aspects, the nucleoside analogs in the ASOs are different. The nucleotide analogs in the ASOs can be any one of or combination of the following nucleoside analogs.

In some aspects, the nucleoside analog comprises a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; bicyclic nucleoside analog; or any combination thereof. In some aspects, the nucleoside analog comprises a sugar modified nucleoside. In some aspects, the nucleoside analog comprises a nucleoside comprising a bicyclic sugar. In some aspects, the nucleoside analog comprises an LNA.

In some aspects, the nucleoside analog is selected from the group consisting of constrained ethyl nucleoside (cEt), 2′,4′-constrained 2′-O-methoxyethyl (cMOE), α-L-LNA, β-D-LNA, 2′-0,4-C-ethylene-bridged nucleic acids (ENA), amino-LNA, oxy-LNA, thio-LNA, and any combination thereof. In some aspects, the ASO comprises one or more 5′-methyl-cytosine nucleobases.

The term nucleobase includes the purine (e.g., adenine and guanine) and pyrimidine (e.g., uracil, thymine and cytosine) moiety present in nucleosides and nucleotides which form hydrogen bonds in nucleic acid hybridization. In the context of the present disclosure, the term nucleobase also encompasses modified nucleobases which may differ from naturally occurring nucleobases, but are functional during nucleic acid hybridization. In some aspects, the nucleobase moiety is modified by modifying or replacing the nucleobase. In this context, “nucleobase” refers to both naturally occurring nucleobases such as adenine, guanine, cytosine, thymidine, uracil, xanthine and hypoxanthine, as well as non-naturally occurring variants. Such variants are for example described in Hirao et al., (2012) Accounts of Chemical Research vol 45 page 2055 and Bergstrom (2009) Current Protocols in Nucleic Acid Chemistry Suppl. 37 1.4.1.

In a some aspects, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl-cytosine, 5-thiozolo-cytosine, 5-propynyl-cytosine, 5-propynyl-uracil, 5-bromouracil, 5-thiazolo-uracil, 2-thio-uracil, 2′thio-thymine, inosine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g., A, T, G, C, or U, wherein each letter may optionally include modified nucleobases of equivalent function. For example, in the exemplified oligonucleotides, the nucleobase moieties are selected from A, T, G, C, and 5-methyl-cytosine. Optionally, for LNA gapmers, 5-methyl-cytosine LNA nucleosides may be used.

The ASO of the disclosure can comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2′ and C4′ carbons on the ribose ring (LNA), or an unlinked ribose ring which typically lacks a bond between the C2′ and C3′ carbons (e.g., UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in RNA nucleosides. Substituents may, for example be introduced at the 2′, 3′, 4′, or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

A 2′ sugar modified nucleoside is a nucleoside which has a substituent other than H or —OH at the 2′ position (2′ substituted nucleoside) or comprises a 2′ linked biradical, and includes 2′ substituted nucleosides and LNA (2′-4′ biradical bridged) nucleosides. For example, the 2′ modified sugar may provide enhanced binding affinity (e.g., affinity enhancing 2′ sugar modified nucleoside) and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acids (ANA), and 2′-Fluoro-ANA nucleoside. For further examples, please see, e.g., Freier & Altmann; Nucl. Acid Res., 1997, 25, 4429-4443; Uhlmann, Curr. Opinion in Drug Development, 2000, 3(2), 293-213; and Deleavey and Damha, Chemistry and Biology 2012, 19, 937. Below are illustrations of some 2′ substituted modified nucleosides.

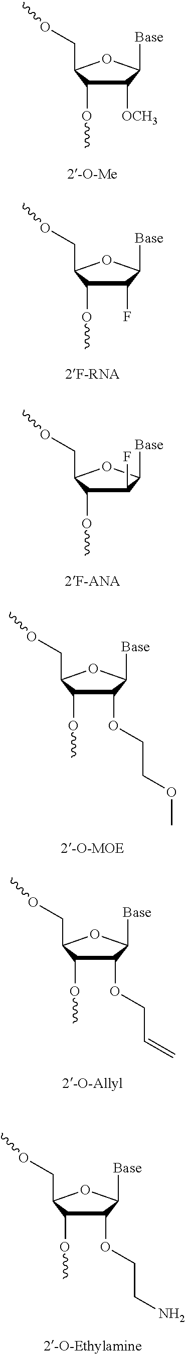

LNA nucleosides are modified nucleosides which comprise a linker group (referred to as a biradical or a bridge) between C2′ and C4′ of the ribose sugar ring of a nucleoside (i.e., 2′-4′ bridge), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med.Chem. Lett. 12, 73-76, Seth et al., J. Org. Chem. 2010, Vol 75(5) pp. 1569-81, and Mitsuoka et al., Nucleic Acids Research 2009, 37(4), 1225-1238.

In some aspects, the modified nucleoside or the LNA nucleosides of the ASO of the disclosure has a general structure of the formula I or II:

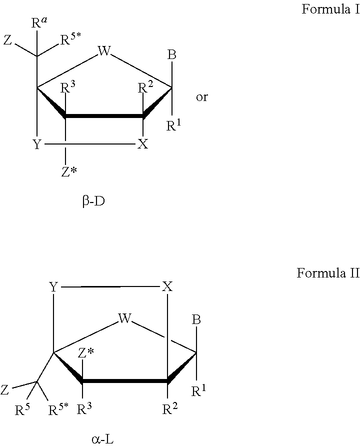

wherein
W is selected from —O—, —S—, —N(R^{a})—, —C(R^{a}R^{b})—, in particular —O—;
B is a nucleobase or a modified nucleobase moiety;
Z is an internucleoside linkage to an adjacent nucleoside or a 5′-terminal group;
Z* is an internucleoside linkage to an adjacent nucleoside or a 3′-terminal group;
R¹, R², R³, R⁵ and R^{5*} are independently selected from hydrogen, halogen, alkyl, alkenyl, alkynyl, hydroxy, alkoxy, alkoxyalkyl, alkenyloxy, carboxyl, alkoxycarbonyl, alkylcarbonyl, formyl, azide, heterocycle and aryl; and
X, Y, R^{a} and R^{b} are as defined herein.

In some aspects, —X—Y—, R^{a} is hydrogen or alkyl, in particular hydrogen or methyl. In some aspects of —X—Y—, R^{b} is hydrogen or alkyl, in particular hydrogen or methyl. In other aspects of —X—Y—, one or both of R^{a} and R^{b} are hydrogen. In further aspects of —X—Y—, only one of R^{a} and R^{b} is hydrogen. In some aspects of —X—Y—, one of R^{a} and R^{b} is methyl and the other one is hydrogen. In certain aspects of —X—Y—, R^{a} and R^{b} are both methyl at the same time.

In some aspects, —X—, R^{a} is hydrogen or alkyl, in particular hydrogen or methyl. In some aspects of —X—, R^{b} is hydrogen or alkyl, in particular hydrogen or methyl. In other aspects of —X—, one or both of R^{a} and R^{b} are hydrogen. In certain aspects of —X—, only one of R^{a} and R^{b} is hydrogen. In certain aspects of —X—, one of R^{a} and R^{b} is methyl and the other one is hydrogen. In other aspects of —X—, R^{a} and R^{b} are both methyl at the same time.

In some aspects, —Y—, R^{a} is hydrogen or alkyl, in particular hydrogen or methyl. In certain aspects of —Y—, R^{b} is hydrogen or alkyl, in particular hydrogen or methyl. In other aspects of —Y—, one or both of R^{a} and R^{b} are hydrogen. In some aspects of —Y—, only one of R^{a} and R^{b} is hydrogen. In other aspects of —Y—, one of R^{a} and R^{b} is methyl and the other one is hydrogen. In some aspects of —Y—, R^{a} and R^{b} are both methyl at the same time.

In some aspects, R¹, R², R³, R⁵ and R^{5*} are independently selected from hydrogen and alkyl, in particular hydrogen and methyl.

In some aspects, R¹, R², R³, R⁵ and R^{5*} are all hydrogen at the same time.

In some aspects, R¹, R², R³, are all hydrogen at the same time, one of R⁵ and R^{5*} is hydrogen and the other one is as defined above, in particular alkyl, more particularly methyl.

In some aspects, R¹, R², R³, are all hydrogen at the same time, one of R⁵ and R^{5*} is hydrogen and the other one is azide.

In some aspects, —X—Y— is —O—CH₂—, W is oxygen and R¹, R², R³, R⁵ and R^{5*} are all hydrogen at the same time. Such LNA nucleosides are disclosed in WO 99/014226, WO 00/66604, WO 98/039352 and WO 2004/046160, which are all hereby incorporated by reference, and include what are commonly known in the art as beta-D-oxy LNA and alpha-L-oxy LNA nucleosides.

In some aspects, —X—Y— is —S—CH₂—, W is oxygen and R¹, R², R³, R⁵ and R^{5*} are all hydrogen at the same time. Such thio LNA nucleosides are disclosed in WO 99/014226 and WO 2004/046160 which are hereby incorporated by reference.

In some aspects, —X—Y— is —NH—CH₂—, W is oxygen and R¹, R², R³, R⁵ and R^{5*} are all hydrogen at the same time. Such amino LNA nucleosides are disclosed in WO 99/014226 and WO 2004/046160, which are hereby incorporated by reference.

In some aspects, —X—Y— is —O—CH₂CH₂— or —OCH₂CH₂CH₂—, W is oxygen, and R¹, R², R³, R⁵ and R^{5*} are all hydrogen at the same time. Such LNA nucleosides are disclosed in WO 00/047599 and Morita et al., Bioorganic & Med.Chem. Lett. 12, 73-76, which are hereby incorporated by reference, and include what are commonly known in the art as 2′-O-4′C-ethylene bridged nucleic acids (ENA).

In some aspects, —X—Y— is —O—CH₂—, W is oxygen, R¹, R², R³ are all hydrogen at the same time, one of R⁵ and R^{5*} is hydrogen and the other one is not hydrogen, such as alkyl, for example methyl. Such 5′ substituted LNA nucleosides are disclosed in WO 2007/134181, which is hereby incorporated by reference.

In some aspects, —X—Y— is —O—CR^{a}R^{b}—, wherein one or both of R^{a} and R^{b} are not hydrogen, in particular alkyl such as methyl, W is oxygen, R¹, R², R³ are all hydrogen at the same time, one of R⁵ and R^{5*} is hydrogen and the other one is not hydrogen, in particular alkyl, for example methyl. Such bis modified LNA nucleosides are disclosed in WO 2010/077578, which is hereby incorporated by reference.

In some aspects, —X—Y— is —O—CH(CH₂—O—CH₃)— (“2′ O-methoxyethyl bicyclic nucleic acid”, Seth et al., J. Org. Chem. 2010, Vol 75(5) pp. 1569-81).

In some aspects, —X—Y— is —O—CHR^{a}—, W is oxygen and R¹, R², R³, R⁵ and R^{5*} are all hydrogen at the same time. Such 6′-substituted LNA nucleosides are disclosed in WO 2010/036698 and WO 2007/090071, which are both hereby incorporated by reference. In such 6′-substituted LNA nucleosides, R^{a} is in particular C1-C6 alkyl, such as methyl.

In some aspects, —X—Y— is —O—CH(CH₂—O—CH₃)—, W is oxygen and R¹, R², R³, R⁵ and R^{5*} are all hydrogen at the same time. Such LNA nucleosides are also known in the art as cyclic MOEs (cMOE) and are disclosed in WO 2007/090071.

In some aspects, —X—Y— is —O—CH(CH₃)—.

In some aspects, —X—Y— is —O—CH₂—O—CH₂— (Seth et al., J. Org. Chem 2010 op. cit.)

In some aspects, —X—Y— is —O—CH(CH₃)—, W is oxygen and R¹, R², R³, R⁵ and R^{5*} are all hydrogen at the same time. Such 6′-methyl LNA nucleosides are also known in the art as cET nucleosides, and may be either (S)-cET or (R)-cET diastereoisomers, as disclosed in WO 2007/090071 (beta-D) and WO 2010/036698 (alpha-L) which are both hereby incorporated by reference.

In some aspects, —X—Y— is —O—CR^{a}R^{b}—, wherein neither R^{a} nor R^{b} is hydrogen, W is oxygen, and R¹, R², R³, R⁵ and R^{5*} are all hydrogen at the same time. In certain aspects, R^{a} and R^{b} are both alkyl at the same time, in particular both methyl at the same time. Such 6′-di-substituted LNA nucleosides are disclosed in WO 2009/006478 which is hereby incorporated by reference.

In some aspects, —X—Y— is —S—CIR^{a}—, W is oxygen, and R¹, R², R³, R⁵ and R^{5*} are all hydrogen at the same time. Such 6′-substituted thio LNA nucleosides are disclosed in WO 2011/156202, which is hereby incorporated by reference. In certain aspects of such 6′-substituted thio LNA, R^{a} is alkyl, in particular methyl.

In some aspects, —X—Y— is —C(═CH₂)C(R^{a}R^{b})—, such as, W is oxygen, and R¹, R², R³, R⁵ and R^{5*} are all hydrogen at the same time. Such vinyl carbo LNA nucleosides are disclosed in WO 2008/154401 and WO 2009/067647, which are both hereby incorporated by reference.

In some aspects, —X—Y— is —N(OR^{a})—CH₂—, W is oxygen and R¹, R², R³, R⁵ and R^{5*} are all hydrogen at the same time. In some aspects, R^{a} is alkyl such as methyl. Such LNA nucleosides are also known as N substituted LNAs and are disclosed in WO 2008/150729, which is hereby incorporated by reference.

In some aspects, —X—Y— is —O—NCH₃— (Seth et al., J. Org. Chem 2010 op. cit.).

In some aspects, —X—Y— is ON(R^{a})— —N(R^{a})—O—, —NR^{a}—CR^{a}R^{b}—CR^{a}R^{b}—, or —NR^{a}—CR^{a}R^{b}—, W is oxygen, and R¹, R², R³, R⁵ and R^{5*} are all hydrogen at the same time. In certain aspects, R^{a} is alkyl, such as methyl. (Seth et al., J. Org. Chem 2010 op. cit.).

In some aspects, R⁵ and R^{5*} are both hydrogen at the same time. In other aspects, one of R⁵ and R^{5*} is hydrogen and the other one is alkyl, such as methyl. In such aspects, R¹, R² and R³ can be in particular hydrogen and —X—Y— can be in particular —O—CH₂— or —O—CHC(R^{a})₃—, such as —O—CH(CH₃)—.

In some aspects, —X—Y— is —CR^{a}R^{b}—O—CR^{a}R^{b}—, such as —CH₂—O—CH₂—, W is oxygen and R¹, R², R³, R⁵ and R^{5*} are all hydrogen at the same time. In such aspects, R^{a} can be in particular alkyl such as methyl. Such LNA nucleosides are also known as conformationally restricted nucleotides (CRNs) and are disclosed in WO 2013/036868, which is hereby incorporated by reference.

In some aspects, —X—Y— is —O—CR^{a}R^{b}—O—CR^{a}R^{b}—, such as —O—CH₂—O—CH₂—, W is oxygen and R¹, R², R³, R⁵ and R^{5*} are all hydrogen at the same time. In certain aspects, R^{a} can be in particular alkyl such as methyl. Such LNA nucleosides are also known as COC nucleotides and are disclosed in Mitsuoka et al., Nucleic Acids Research 2009, 37(4), 1225-1238, which is hereby incorporated by reference.

It will be recognized than, unless specified, the LNA nucleosides may be in the beta-D or alpha-L stereoisoform.

Certain examples of LNA nucleosides are presented in Scheme 1.

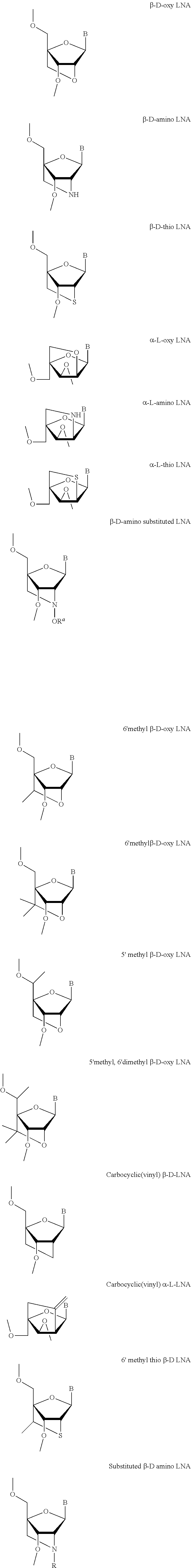

As illustrated elsewhere, in some aspects of the disclosure the LNA nucleosides in the oligonucleotides are beta-D-oxy-LNA nucleosides.

### II.A.5. Nuclease Mediated Degradation

Nuclease mediated degradation refers to an oligonucleotide capable of mediating degradation of a complementary nucleotide sequence when forming a duplex with such a sequence.

In some aspects, the oligonucleotide may function via nuclease mediated degradation of the target nucleic acid, where the oligonucleotides of the disclosure are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example gapmers.

### II.A.6. RNase H Activity and Recruitment

The RNase H activity of an antisense oligonucleotide refers to its ability to recruit RNase H when in a duplex with a complementary RNA molecule and induce degradation of the complementary RNA molecule. WO01/23613 provides in vitro methods for determining RNaseH activity, which may be used to determine the ability to recruit RNaseH. Typically, an oligonucleotide is deemed capable of recruiting RNase H if, when provided with a complementary target nucleic acid sequence, it has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers, with phosphorothioate linkages between all monomers in the oligonucleotide, and using the methodology provided by Example 91-95 of WO01/23613.

In some aspects, an oligonucleotide is deemed essentially incapable of recruiting RNaseH if, when provided with the complementary target nucleic acid, the RNaseH initial rate, as measured in pmol/l/min, is less than 20%, such as less than 10%,such as less than 5% of the initial rate determined when using a oligonucleotide having the same base sequence as the oligonucleotide being tested, but containing only DNA monomers, with no 2′ substitutions, with phosphorothioate linkages between all monomers in the oligonucleotide, and using the methodology provided by Example 91-95 of WO01/23613.

### II.A.7. ASO Design

The ASO of the disclosure can comprise a nucleotide sequence which comprises both nucleosides and nucleoside analogs, and can be in the form of a gapmer. Examples of configurations of a gapmer that can be used with the ASO of the disclosure are described in U.S. Patent Appl. Publ. No. 2012/0322851.

The term “gapmer” as used herein refers to an antisense oligonucleotide which comprises a region of RNase H recruiting oligonucleotides (gap) which is flanked 5′ and 3′ by one or more affinity enhancing modified nucleosides (flanks). The term “LNA gapmer” is a gapmer oligonucleotide wherein at least one of the affinity enhancing modified nucleosides is an LNA nucleoside. The term “mixed wing gapmer” refers to an LNA gapmer wherein the flank regions comprise at least one LNA nucleoside and at least one DNA nucleoside or non-LNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acid (ANA), and 2′-Fluoro-ANA nucleoside(s).

In some aspects, the ASO of the disclosure can be in the form of a mixmer. In some aspects, the ASO of the disclosure can be in the form of a totalmer. In some aspects, in addition to enhancing affinity of the ASO for the target region, some nucleoside analogs also mediate RNase (e.g., RNaseH) binding and cleavage. Since a-L-LNA monomers recruit RNaseH activity to a certain extent, in some aspects, gap regions (e.g., region B as referred to herein) of ASOs containing a-L-LNA monomers consist of fewer monomers recognizable and cleavable by the RNaseH, and more flexibility in the mixmer construction is introduced.

In some aspects, the ASO of the disclosure is a gapmer and comprises a contiguous stretch of nucleotides (e.g., one or more DNA) which is capable of recruiting an RNase, such as RNaseH, referred to herein in as region B (B), wherein region B is flanked at both 5′ and 3′ by regions of nucleoside analogs 5′ and 3′ to the contiguous stretch of nucleotides of region B— these regions are referred to as regions A (A) and C (C), respectively. In some aspects, the nucleoside analogs are sugar modified nucleosides (e.g., high affinity sugar modified nucleosides). In certain aspects, the sugar modified nucleosides of regions A and C enhance the affinity of the ASO for the target nucleic acid (i.e., affinity enhancing 2′ sugar modified nucleosides). In some aspects, the sugar modified nucleosides are 2′ sugar modified nucleosides, such as high affinity 2′ sugar modifications, such as LNA and/or 2′-MOE.

In a gapmer, the 5′ and 3′ most nucleosides of region B are DNA nucleosides, and are positioned adjacent to nucleoside analogs (e.g., high affinity sugar modified nucleosides) of regions A and C, respectively. In some aspects, regions A and C can be further defined by having nucleoside analogs at the end most distant from region B (i.e., at the 5′ end of region A and at the 3′ end of region C).

In some aspects, the ASOs of the present disclosure comprise a nucleotide sequence of formula (5′ to 3′) A-B-C, wherein: (A) (5′ region or a first wing sequence) comprises at least one nucleoside analog (e.g., 3-5 LNA units); (B) comprises at least four consecutive nucleosides (e.g., 4-24 DNA units), which are capable of recruiting RNase (when formed in a duplex with a complementary RNA molecule, such as the pre-mRNA or mRNA target); and (C) (3′ region or a second wing sequence) comprises at least one nucleoside analog (e.g., 3-5 LNA units).

In some aspects, region A comprises 3-5 nucleoside analogs, such as LNA, region B consists of 6-24 (e.g., 6, 7, 8, 9, 10, 11, 12, 13, or 14) DNA units, and region C consists of 3 or 4 nucleoside analogs, such as LNA. Such designs include (A-B-C) 3-14-3, 3-11-3, 3-12-3, 3-13-3, 4-9-4, 4-10-4, 4-11-4, 4-12-4, and 5-10-5. In some aspects, the ASO has a design of LLLDₙLLL, LLLLDₙLLLL, or LLLLLDₙLLLLL, wherein the L is a nucleoside analog, the D is DNA, and n can be any integer between 4 and 24. In some aspects, n can be any integer between 6 and 14. In some aspects, n can be any integer between 8 and 12. In some aspects, the ASO has a design of LLLMMDnMMLLL, LLLMDnMLLL, LLLLMMDnMMLLLL, LLLLMDnMLLLL, LLLLLLMMDnMMLLLLL, or LLLLLLMDnMLLLLL, wherein the D is DNA, n can be any integer between 3 and 15, the L is LNA, and the M is 2′MOE.

Further gapmer designs are disclosed in WO2004/046160, WO 2007/146511, and WO2008/113832, each of which is hereby incorporated by reference in its entirety.

### II.A.9. Internucleotide Linkages

The monomers of the ASOs described herein are coupled together via linkage groups. Suitably, each monomer is linked to the 3′ adjacent monomer via a linkage group.

The person having ordinary skill in the art would understand that, in the context of the present disclosure, the 5′ monomer at the end of an ASO does not comprise a 5′ linkage group, although it may or may not comprise a 5′ terminal group.

In some aspects, the contiguous nucleotide sequence comprises one or more modified internucleoside linkages. The terms “linkage group” or “internucleoside linkage” are intended to mean a group capable of covalently coupling together two nucleosides. Non-limiting examples include phosphate groups and phosphorothioate groups.

The nucleosides of the ASO of the disclosure or contiguous nucleosides sequence thereof are coupled together via linkage groups. Suitably, each nucleoside is linked to the 3′ adjacent nucleoside via a linkage group.

In some aspects, the internucleoside linkage is modified from its normal phosphodiester to one that is more resistant to nuclease attack, such as phosphorothioate, which is cleavable by RNaseH, also allows that route of antisense inhibition in reducing the expression of the target gene. In some aspects, at least 75%, at least 80%, at least 85%, at least 90%, at least 91%, at least 92%, at least 93%, at least 94%, at least 95%, at least 96%, at least 97%, at least 98%, at least 99%, or 100% of internucleoside linkages are modified.

### II.B. pH

In some aspects, the pharmaceutical composition has a pH that can stably formulate an EV. The pH can be in a range of about 7.0 to about 7.4, e.g., about 7.1 to about 7.3, e.g., about 7.2. In some aspects, the composition is in a solution at a pH of about 7.2. The EVs are disclosed elsewhere herein, the saccharide can be a monosaccharide, a disaccharide, a trisaccharide, or any other saccharides; sodium chloride is shown below; and the potassium and sodium phosphates are shown below.

In some aspects, the composition prior to freezing and after the freezing remains the same. For example, the composition prior to freezing and after the freezing has a pH of about 7.1. In some aspects, the composition prior to freezing and after the freezing has a pH of about 7.2. In some aspects, the composition prior to freezing and after the freezing has a pH of about 7.3. In some aspects, the composition prior to freezing and after the freezing has a pH of about 7.4.

In some aspects, the pH of the composition can be adjusted by modifying the concentration of phosphates. In some aspects, the pH of the composition can be adjusted by modifying the concentration of the potassium phosphate. In some aspects, the pH of the composition can be increased by adding or increasing the concentration of a potassium phosphate. In some aspects, the concentration of the potassium phosphate is higher than the concentration of the sodium phosphate.

In some aspects, the ratio of the monobasic and dibasic forms of sodium phosphate and potassium phosphate can be used to adjust the pH of a pharmaceutical composition. In some aspects, sodium phosphate monobasic and/or potassium phosphate monobasic can be used to increase the pH of a pharmaceutical composition. In some aspects, sodium phosphate dibasic and/or potassium phosphate dibasic can be used to decrease the pH of a pharmaceutical composition. in some aspects, pH ranges for the disclosed compositions are between about 6.8 to about 7.6. Therefore, if the pH of the composition is lower than desired, the pH can be changed by changing the ratio of monobasic to dibasic form of the salt, (i.e., of potassium or sodium). In some aspects, the ratio of the monobasic and dibasic forms of sodium phosphate and potassium phosphate can be used to adjust the pH of the composition until the pH of the composition is between 7.0 and 7.4, e.g., 7.1 and 7.3, e.g., 7.2. In some aspects, the upper limit of pH is due to the destruction of the lipids of the disclosed EVs, e.g., exosomes, which undergo hydrolysis more commonly known as saponification. In some aspects, the potassium salts stabilize the pH upon freezing.

### II.C. Sodium Chloride

In some aspects, the sodium chloride is present in the composition at a concentration of between about 10 mM and about 200 mM sodium chloride. In some aspects, the sodium chloride is present in the composition at a concentration of between about 10 mM and about 134 mM, between about 10 mM to about 190 mM, between about 20 mM to about 180 mM, between about 30 mM to about 170 mM, between about 40 mM to about 160 mM, between about 50 mM to about 150 mM, between about 60 mM to about 140 mM, between about 70 mM to about 130 mM, between about 80 mM to about 120 mM, between about 90 mM to about 110 mM, between about 95 mM to about 105 mM, between about 90 mM to about 100 mM, between about 100 mM to about 110 mM, between about 95 mM to about 100 mM, between about 100 mM to about 105 mM, between about 50 mM to about 140 mM, between about 60 mM to about 130 mM, between about 70 mM to about 120 mM, between about 80 mM to about 110 mM, between about 95 mM to about 110 mM, or between about 90 mM to about 105 mM. In some aspects, the concentration of sodium chloride is between about 50 mM and about 150 mM. In some aspects, the concentration of sodium chloride is between about 50 mM and about 140 mM. In some aspects, the concentration of sodium chloride is between about 60 mM to about 140 mM. In some aspects, the concentration of sodium chloride is between about 60 mM to about 130 mM. In some aspects, the concentration of sodium chloride is between about 70 mM to about 130 mM. In some aspects, the concentration of sodium chloride is between about 70 mM to about 120 mM. In some aspects, the concentration of sodium chloride is between about 80 mM to about 120 mM. In some aspects, the concentration of sodium chloride is between about 80 mM to about 110 mM. In some aspects, the concentration of sodium chloride is between about 90 mM to about 110 mM. In some aspects, the concentration of sodium chloride is between about 90 mM to about 105 mM. In some aspects, the concentration of sodium chloride is between about 90 mM to about 100 mM. In some aspects, the concentration of sodium chloride is between about 95 mM to about 110 mM. In some aspects, the concentration of sodium chloride is between about 95 mM to about 105 mM. In some aspects, the concentration of sodium chloride is between about 95 mM to about 100 mM.

In some aspects, the concentration of sodium chloride is about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, or about 150 mM. In some aspects, the concentration of sodium chloride is about 50 mM. In some aspects, the concentration of sodium chloride is about 60 mM. In some aspects, the concentration of sodium chloride is about 70 mM. In some aspects, the concentration of sodium chloride is about 80 mM. In some aspects, the concentration of sodium chloride is about 90 mM. In some aspects, the concentration of sodium chloride is about 95 mM. In some aspects, the concentration of sodium chloride is about 100 mM. In some aspects, the concentration of sodium chloride is about 105 mM. In some aspects, the concentration of sodium chloride is about 110 mM. In some aspects, the concentration of sodium chloride is about 120 mM. In some aspects, the concentration of sodium chloride is about 130 mM. In some aspects, the concentration of sodium chloride is about 140 mM. In some aspects, the concentration of sodium chloride is about 150 mM.

In some aspects, the concentration of sodium chloride is at least about 2.9 mg/ml. In some aspects, the concentration of sodium chloride is at least about 3.0 mg/ml. In some aspects, the concentration of sodium chloride is at least about 3.1 mg/ml. In some aspects, the concentration of sodium chloride is at least about 3.2 mg/ml. In some aspects, the concentration of sodium chloride is at least about 3.3 mg/ml. In some aspects, the concentration of sodium chloride is at least about 3.4 mg/ml. In some aspects, the concentration of sodium chloride is at least about 3.5 mg/ml. In some aspects, the concentration of sodium chloride is at least about 3.6 mg/ml. In some aspects, the concentration of sodium chloride is at least about 3.7 mg/ml. In some aspects, the concentration of sodium chloride is at least about 3.8 mg/ml. In some aspects, the concentration of sodium chloride is at least about 3.9 mg/ml. In some aspects, the concentration of sodium chloride is at least about 4.0 mg/ml. In some aspects, the concentration of sodium chloride is at least about 4.1 mg/ml. In some aspects, the concentration of sodium chloride is at least about 4.2 mg/ml. In some aspects, the concentration of sodium chloride is at least about 4.3 mg/ml. In some aspects, the concentration of sodium chloride is at least about 4.4 mg/ml. In some aspects, the concentration of sodium chloride is at least about 4.5 mg/ml. In some aspects, the concentration of sodium chloride is at least about 4.6 mg/ml. In some aspects, the concentration of sodium chloride is at least about 4.7 mg/ml. In some aspects, the concentration of sodium chloride is at least about 4.8 mg/ml. In some aspects, the concentration of sodium chloride is at least about 4.9 mg/ml. In some aspects, the concentration of sodium chloride is at least about 5.0 mg/ml. In some aspects, the concentration of sodium chloride is at least about 5.1 mg/ml. In some aspects, the concentration of sodium chloride is at least about 5.2 mg/ml. In some aspects, the concentration of sodium chloride is at least about 5.3 mg/ml. In some aspects, the concentration of sodium chloride is at least about 5.4 mg/ml. In some aspects, the concentration of sodium chloride is at least about 5.5 mg/ml. In some aspects, the concentration of sodium chloride is at least about 5.6 mg/ml. In some aspects, the concentration of sodium chloride is at least about 5.7 mg/ml. In some aspects, the concentration of sodium chloride is at least about 5.8 mg/ml. In some aspects, the concentration of sodium chloride is at least about 5.9 mg/ml. In some aspects, the concentration of sodium chloride is at least about 6.0 mg/ml. In some aspects, the concentration of sodium chloride is at least about 6.1 mg/ml. In some aspects, the concentration of sodium chloride is at least about 6.2 mg/ml. In some aspects, the concentration of sodium chloride is at least about 6.3 mg/ml. In some aspects, the concentration of sodium chloride is at least about 6.4 mg/ml. In some aspects, the concentration of sodium chloride is at least about 6.5 mg/ml. In some aspects, the concentration of sodium chloride is at least about 6.6 mg/ml. In some aspects, the concentration of sodium chloride is at least about 6.7 mg/ml. In some aspects, the concentration of sodium chloride is at least about 6.8 mg/ml. In some aspects, the concentration of sodium chloride is at least about 6.9 mg/ml. In some aspects, the concentration of sodium chloride is at least about 7.0 mg/ml.

### II.D. Phosphates

In some aspects, a potassium phosphate, e.g., potassium phosphate monobasic, is present in the composition at a concentration of between about 1 mM to about 20 mM, about 1 mM to about 10 mM, about 2 mM to about 10 mM, about 3 mM to about 10 mM, about 4 mM to about 10 mM, about 5 mM to about 10 mM, about 2 mM to about 9 mM, about 3 mM to about 8 mM, about 4 mM to about 7 mM, about 4 mM to about 6 mM, about 3 mM to about 6 mM, or about 4.5 mM to about 5.5 mM.

In some aspects, the concentration of the potassium phosphate, e.g., potassium phosphate monobasic, is about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, or about 5.5 mM.

In some aspects, the potassium phosphate is present in the composition at a concentration of between about 1 mM to about 20 mM. In some aspects, the potassium phosphate is present in the composition at a concentration of between about 1 mM to about 10 mM. In some aspects, the potassium phosphate is present in the composition at a concentration of between about 2 mM to about 10 mM. In some aspects, the potassium phosphate is present in the composition at a concentration of between about 3 mM to about 10 mM. In some aspects, the potassium phosphate is present in the composition at a concentration of between about 4 mM to about 10 mM. In some aspects, the potassium phosphate is present in the composition at a concentration of between about 5 mM to about 10 mM. In some aspects, the potassium phosphate is present in the composition at a concentration of between about 2 mM to about 9 mM. In some aspects, the potassium phosphate is present in the composition at a concentration of between about 3 mM to about 8 mM. In some aspects, the potassium phosphate is present in the composition at a concentration of between about 4 mM to about 7 mM. In some aspects, the potassium phosphate is present in the composition at a concentration of between about 4 mM to about 6 mM. In some aspects, the potassium phosphate is present in the composition at a concentration of between about 3 mM to about 6 mM. In some aspects, the potassium phosphate is present in the composition at a concentration of between about 4.5 mM to about 5.5 mM.

In some aspects, the potassium phosphate, e.g., potassium phosphate monobasic, is present in the composition at a concentration of about 3.0 mM. In some aspects, the potassium phosphate, e.g., potassium phosphate monobasic, is present in the composition at a concentration of about 3.5 mM. In some aspects, the potassium phosphate, e.g., potassium phosphate monobasic, is present in the composition at a concentration of about 4.0 mM. In some aspects, the potassium phosphate, e.g., potassium phosphate monobasic, is present in the composition at a concentration of about 4.1 mM. In some aspects, the potassium phosphate, e.g., potassium phosphate monobasic, is present in the composition at a concentration of about 4.2 mM. In some aspects, the potassium phosphate, e.g., potassium phosphate monobasic, is present in the composition at a concentration of about 4.3 mM. In some aspects, the potassium phosphate, e.g., potassium phosphate monobasic, is present in the composition at a concentration of about 4.4 mM. In some aspects, the potassium phosphate, e.g., potassium phosphate monobasic, is present in the composition at a concentration of about 4.5 mM. In some aspects, the potassium phosphate is present in the composition at a concentration of about 4.6 mM. In some aspects, the potassium phosphate is present in the composition at a concentration of about 4.7 mM. In some aspects, the potassium phosphate is present in the composition at a concentration of about 4.8 mM. In some aspects, the potassium phosphate is present in the composition at a concentration of about 4.9 mM. In some aspects, the potassium phosphate is present in the composition at a concentration of about 5.0 mM. In some aspects, the potassium phosphate is present in the composition at a concentration of about 5.1 mM. In some aspects, the potassium phosphate is present in the composition at a concentration of about 5.2 mM. In some aspects, the potassium phosphate is present in the composition at a concentration of about 5.3 mM. In some aspects, the potassium phosphate is present in the composition at a concentration of about 5.4 mM. In some aspects, the potassium phosphate is present in the composition at a concentration of about 5.5 mM. In some aspects, the potassium phosphate is present in the composition at a concentration of about 5.6 mM. In some aspects, the potassium phosphate is present in the composition at a concentration of about 5.7 mM. In some aspects, the potassium phosphate is present in the composition at a concentration of about 5.8 mM. In some aspects, the potassium phosphate is present in the composition at a concentration of about 5.9 mM. In some aspects, the potassium phosphate is present in the composition at a concentration of about 6.0 mM. In some aspects, the concentration of the potassium phosphate in the composition is 5.15 mM.

Any of the concentrations of potassium phosphate monobasic disclosed herein can be expressed in terms of weight per volume, e.g., mg/ml. A person of ordinary skill would be able to readily convert the mM concentrations disclosed herein to weight per volume concentrations. In some aspects, the concentration of the potassium phosphate, e.g., potassium phosphate monobasic, is from at least about 0.14 mg/ml to at least about 2.75 mg/ml. In some aspects, the concentration of the potassium phosphate, e.g., potassium phosphate monobasic, is at least about 0.14 mg/ml. In some aspects, the concentration of the potassium phosphate, e.g., potassium phosphate monobasic, is at least about 0.15 mg/ml. In some aspects, the concentration of the potassium phosphate, e.g., potassium phosphate monobasic, is at least about 0.17 mg/ml. In some aspects, the concentration of the potassium phosphate, e.g., potassium phosphate monobasic, is at least about 0.2 mg/ml. In some aspects, the concentration of the potassium phosphate, e.g., potassium phosphate monobasic, is at least about 0.23 mg/ml. In some aspects, the concentration of the potassium phosphate, e.g., potassium phosphate monobasic, is at least about 0.25 mg/ml. In some aspects, the concentration of the potassium phosphate, e.g., potassium phosphate monobasic, is at least about 0.5 mg/ml. In some aspects, the concentration of the potassium phosphate, e.g., potassium phosphate monobasic, is at least about 0.60 mg/ml. In some aspects, the concentration of the potassium phosphate, e.g., potassium phosphate monobasic, is at least about 0.61 mg/ml. In some aspects, the concentration of the potassium phosphate, e.g., potassium phosphate monobasic, is at least about 0.62 mg/ml. In some aspects, the concentration of the potassium phosphate, e.g., potassium phosphate monobasic, is at least about 0.63 mg/ml. In some aspects, the concentration of the potassium phosphate, e.g., potassium phosphate monobasic, is at least about 0.64 mg/ml. In some aspects, the concentration of the potassium phosphate, e.g., potassium phosphate monobasic, is at least about 0.65 mg/ml. In some aspects, the concentration of the potassium phosphate, e.g., potassium phosphate monobasic, is at least about 0.66 mg/ml. In some aspects, the concentration of the potassium phosphate, e.g., potassium phosphate monobasic, is at least about 0.67 mg/ml. In some aspects, the concentration of the potassium phosphate, e.g., potassium phosphate monobasic, is at least about 0.68 mg/ml. In some aspects, the concentration of the potassium phosphate, e.g., potassium phosphate monobasic, is at least about 0.69 mg/ml. In some aspects, the concentration of the potassium phosphate, e.g., potassium phosphate monobasic, is at least about 0.70 mg/ml. In some aspects, the concentration of the potassium phosphate, e.g., potassium phosphate monobasic, is at least about 0.71 mg/ml. In some aspects, the concentration of the potassium phosphate, e.g., potassium phosphate monobasic, is at least about 0.72 mg/ml. In some aspects, the concentration of the potassium phosphate, e.g., potassium phosphate monobasic, is at least about 0.73 mg/ml. In some aspects, the concentration of the potassium phosphate, e.g., potassium phosphate monobasic, is at least about 0.74 mg/ml. In some aspects, the concentration of the potassium phosphate, e.g., potassium phosphate monobasic, is at least about 0.75 mg/ml. In some aspects, the concentration of the potassium phosphate, e.g., potassium phosphate monobasic, is at least about 1.0 mg/ml. In some aspects, the concentration of the potassium phosphate, e.g., potassium phosphate monobasic, is at least about 1.25 mg/ml. In some aspects, the concentration of the potassium phosphate, e.g., potassium phosphate monobasic, is at least about 1.50 mg/ml. In some aspects, the concentration of the potassium phosphate, e.g., potassium phosphate monobasic, is at least about 1.75 mg/ml. In some aspects, the concentration of the potassium phosphate, e.g., potassium phosphate monobasic, is at least about 2.0 mg/ml. In some aspects, the concentration of the potassium phosphate, e.g., potassium phosphate monobasic, is at least about 2.03 mg/ml. In some aspects, the concentration of the potassium phosphate, e.g., potassium phosphate monobasic, is at least about 2.04 mg/ml. In some aspects, the concentration of the potassium phosphate, e.g., potassium phosphate monobasic, is at least about 2.05 mg/ml. In some aspects, the concentration of the potassium phosphate, e.g., potassium phosphate monobasic, is at least about 2.1 mg/ml. In some aspects, the concentration of the potassium phosphate, e.g., potassium phosphate monobasic, is at least about 2.2 mg/ml. In some aspects, the concentration of the potassium phosphate, e.g., potassium phosphate monobasic, is at least about 2.3 mg/ml. In some aspects, the concentration of the potassium phosphate, e.g., potassium phosphate monobasic, is at least about 2.4 mg/ml. In some aspects, the concentration of the potassium phosphate, e.g., potassium phosphate monobasic, is at least about 2.5 mg/ml. In some aspects, the concentration of the potassium phosphate, e.g., potassium phosphate monobasic, is at least about 2.6 mg/ml. In some aspects, the concentration of the potassium phosphate, e.g., potassium phosphate monobasic, is at least about 2.7 mg/ml. In some aspects, the concentration of the potassium phosphate, e.g., potassium phosphate monobasic, is at least about 2.8 mg/ml. In some aspects, the concentration of the potassium phosphate, e.g., potassium phosphate monobasic, is at least about 2.9 mg/ml. In some aspects, the concentration of the potassium phosphate, e.g., potassium phosphate monobasic, is at least about 3.0 mg/ml.

In some aspects, the sodium phosphate, e.g., sodium phosphate dibasic heptahydrate, is present in the composition at a concentration of between about 5 mM to about 100 mM, 5 mM to about 30, between about 10 mM to about 20 mM, between about 11 mM to about 19 mM, between about 12 mM to about 18 mM, between about 13 mM to about 17 mM, between about 14 mM to about 16 mM, between about 15 mM to about 16 mM, or between about 14 mM to about 15 mM. In some aspects, the sodium phosphate is present in the composition at a concentration of between about 11 mM to about 19 mM. In some aspects, the sodium phosphate is present in the composition at a concentration of between about 12 mM to about 18 mM. In some aspects, the sodium phosphate is present in the composition at a concentration of between about 13 mM to about 17 mM. In some aspects, the sodium phosphate is present in the composition at a concentration of between about 14 mM to about 16 mM.

In some aspects, the sodium phosphate is present in the composition at a concentration of about 14.5 mM, about 14.6 mM, about 14.7 mM, about 14.8 mM, about 14.9 mM, about 15.0 mM, about 15.1 mM about 15.2 mM, about 15.3 mM, about 15.4, mM, or about 15.5 mM. In some aspects, the sodium phosphate is present in the composition at a concentration of about 14.5 mM. In some aspects, the sodium phosphate is present in the composition at a concentration of about 14.6 mM. In some aspects, the sodium phosphate is present in the composition at a concentration of about 14.7 mM. In some aspects, the sodium phosphate is present in the composition at a concentration of about 14.8 mM. In some aspects, the sodium phosphate is present in the composition at a concentration of about 14.9 mM. In some aspects, the sodium phosphate is present in the composition at a concentration of about 15.0 mM. In some aspects, the sodium phosphate is present in the composition at a concentration of about 15.1 mM. In some aspects, the sodium phosphate is present in the composition at a concentration of about 15.2 mM. In some aspects, the sodium phosphate is present in the composition at a concentration of, about 15.3 mM. In some aspects, the sodium phosphate is present in the composition at a concentration of about 15.4 mM. In some aspects, the sodium phosphate is present in the composition at a concentration of about 15.5 mM. In some aspects, the concentration of the sodium phosphate is 14.9 mM.

Any of the concentrations of sodium phosphate monobasic herein can be expressed in terms of weight per volume, e.g., mg/ml. A person of ordinary skill would be able to readily convert the mM concentrations disclosed herein to weight per volume concentrations. In some aspects, the sodium phosphate is present in the composition at a concentration from at least about 1.42 mg/ml to at least about 14.2 mg/ml. In some aspects, the sodium phosphate is present in the composition at a concentration of at least about 1.4 mg/ml. In some aspects, the sodium phosphate is present in the composition at a concentration of at least about 1.5 mg/ml. In some aspects, the sodium phosphate is present in the composition at a concentration of at least about 1.6 mg/ml. In some aspects, the sodium phosphate is present in the composition at a concentration of at least about 1.7 mg/ml. In some aspects, the sodium phosphate is present in the composition at a concentration of at least about 1.8 mg/ml. In some aspects, the sodium phosphate is present in the composition at a concentration of at least about 1.9 mg/ml. In some aspects, the sodium phosphate is present in the composition at a concentration of at least about 2.0 mg/ml. In some aspects, the sodium phosphate is present in the composition at a concentration of at least about 2.1 mg/ml. In some aspects, the sodium phosphate is present in the composition at a concentration of at least about 2.13 mg/ml. In some aspects, the sodium phosphate is present in the composition at a concentration of at least about 2.2 mg/ml. In some aspects, the sodium phosphate is present in the composition at a concentration of at least about 2.25 mg/ml. In some aspects, the sodium phosphate is present in the composition at a concentration of at least about 2.3 mg/ml. In some aspects, the sodium phosphate is present in the composition at a concentration of at least about 2.4 mg/ml. In some aspects, the sodium phosphate is present in the composition at a concentration of at least about 2.5 mg/ml. In some aspects, the sodium phosphate is present in the composition at a concentration of at least about 2.6 mg/ml. In some aspects, the sodium phosphate is present in the composition at a concentration of at least about 2.7 mg/ml. In some aspects, the sodium phosphate is present in the composition at a concentration of at least about 2.75 mg/ml. In some aspects, the sodium phosphate is present in the composition at a concentration of at least about 2.8 mg/ml. In some aspects, the sodium phosphate is present in the composition at a concentration of at least about 2.9 mg/ml. In some aspects, the sodium phosphate is present in the composition at a concentration of at least about 3.0 mg/ml. In some aspects, the sodium phosphate is present in the composition at a concentration of at least about 3.25 mg/ml. In some aspects, the sodium phosphate is present in the composition at a concentration of at least about 3.5 mg/ml. In some aspects, the sodium phosphate is present in the composition at a concentration of at least about 3.75 mg/ml. In some aspects, the sodium phosphate is present in the composition at a concentration of at least about 3.8 mg/ml. In some aspects, the sodium phosphate is present in the composition at a concentration of at least about 3.83 mg/ml. In some aspects, the sodium phosphate is present in the composition at a concentration of at least about 3.85 mg/ml. In some aspects, the sodium phosphate is present in the composition at a concentration of at least about 3.9 mg/ml. In some aspects, the sodium phosphate is present in the composition at a concentration of at least about 4.0 mg/ml. In some aspects, the sodium phosphate is present in the composition at a concentration of at least about 4.25 mg/ml. In some aspects, the sodium phosphate is present in the composition at a concentration of at least about 4.5 mg/ml. In some aspects, the sodium phosphate is present in the composition at a concentration of at least about 4.75 mg/ml. In some aspects, the sodium phosphate is present in the composition at a concentration of at least about 5.0 mg/ml. In some aspects, the sodium phosphate is present in the composition at a concentration of at least about 5.5 mg/ml. In some aspects, the sodium phosphate is present in the composition at a concentration of at least about 6.0 mg/ml. In some aspects, the sodium phosphate is present in the composition at a concentration of at least about 6.5 mg/ml. In some aspects, the sodium phosphate is present in the composition at a concentration of at least about 7.0 mg/ml. In some aspects, the sodium phosphate is present in the composition at a concentration of at least about 7.5 mg/ml. In some aspects, the sodium phosphate is present in the composition at a concentration of at least about 8.0 mg/ml. In some aspects, the sodium phosphate is present in the composition at a concentration of at least about 8.5 mg/ml. In some aspects, the sodium phosphate is present in the composition at a concentration of at least about 9.0 mg/ml. In some aspects, the sodium phosphate is present in the composition at a concentration of at least about 9.5 mg/ml. In some aspects, the sodium phosphate is present in the composition at a concentration of at least about 10.0 mg/ml. In some aspects, the sodium phosphate is present in the composition at a concentration of at least about 11.0 mg/ml. In some aspects, the sodium phosphate is present in the composition at a concentration of at least about 12.0 mg/ml. In some aspects, the sodium phosphate is present in the composition at a concentration of at least about 13.0 mg/ml. In some aspects, the sodium phosphate is present in the composition at a concentration of at least about 14.0 mg/ml. In some aspects, the sodium phosphate is present in the composition at a concentration of at least about 14.0 mg/ml.

### II.E. Saccharides

In some aspects, the saccharide present in the composition is a monosaccharide, a disaccharide, a trisaccharide, or any other saccharide. In some aspects, the saccharide is a sucrose. In some aspects, the saccharide is a trehalose.

The suitable amount of sucrose or trehalose in the composition stabilizes the composition and/or reduces any aggregates. In some aspects, the pharmaceutical composition comprises (i) an extracellular vesicle and (ii) a saccharide, which is a sucrose or a trehalose at a concentration of about 5% w/v.

The saccharide disclosed herein, e.g., a sucrose or a trehalose, at the concentration of 5% w/v, can provide superior stability to a composition comprising 1% w/v sucrose. In particular, pharmaceutical compositions comprising (i) an extracellular vesicle, e.g., exosome and (ii) a saccharide, which is a sucrose or a trehalose at a concentration of about 5% w/v, provide advantages, including, but not limited to: (i) reduced aggregation of EVs, (ii) improved stability of EVs, (iii) improved integrity of the EV architecture, (iv) improved stability of engineered proteins contained on or in EVs, and (v) improved stability of passively loaded or conjugated materials such as small molecule drugs or proteins. In some aspects, the advantages further include improved filterability and reduced dissociation rates of ASO. In some aspects, the composition has reduced aggregation compared to a reference composition comprising a sucrose or a trehalose at a concentration of 1% w/v to 4% w/v, e.g., 1%. In some aspects, the composition has improved stability compared to a reference composition comprising a sucrose or a trehalose at a concentration of 1% w/v to 4% w/v, e.g., 1%. In some aspects, the composition has improved integrity of the EV architecture compared to a reference composition comprising a sucrose or a trehalose at a concentration of 1% w/v to 4% w/v, e.g., 1%.

In some aspects, the composition has improved stability of engineered proteins contained on or in EVs compared to a reference composition comprising a sucrose or a trehalose at a concentration of 1% w/v to 4% w/v, e.g., 1%.

In some aspects, the composition has improved stability of passively loaded or conjugated materials such as small molecule drugs or proteins, compared to a reference composition comprising a sucrose or a trehalose at a concentration of 1% w/v to 4% w/v, e.g., 1%.

In other aspects, the composition has improved stability compared to a reference composition comprising an sucrose or a trehalose at a concentration higher than 5.5% w/v, 6% w/v, 70% w/v, 80% w/v, 9% w/v, or 10% w/v.

In some aspects, the saccharide, e.g., sucrose or trehalose, is present in the composition at a concentration from at least about 1% to at least about 10%, from at least about 2% to at least about 9%, from at least about 3% to at least about 8%, from at least about 4% to at least about 7%, from at least about 4% to at least about 6%, from at least about 3% to at least about 7%, from at least about 5% to at least about 10%, from at least about 5% to at least about 9%, from at least about 5% to at least about 8%, or from at least about 5% to at least about 7%. In some aspects, the saccharide, e.g., sucrose or trehalose, is present in the composition at a concentration of at least about 1%. In some aspects, the saccharide, e.g., sucrose or trehalose, is present in the composition at a concentration of at least about 2%. In some aspects, the saccharide, e.g., sucrose or trehalose, is present in the composition at a concentration of at least about 3%. In some aspects, the saccharide, e.g., sucrose or trehalose, is present in the composition at a concentration of at least about 4%. In some aspects, the saccharide, e.g., sucrose or trehalose, is present in the composition at a concentration of at least about 5%. In some aspects, the saccharide, e.g., sucrose or trehalose, is present in the composition at a concentration of at least about 6%. In some aspects, the saccharide, e.g., sucrose or trehalose, is present in the composition at a concentration of at least about 7%. In some aspects, the saccharide, e.g., sucrose or trehalose, is present in the composition at a concentration of at least about 8%. In some aspects, the saccharide, e.g., sucrose or trehalose, is present in the composition at a concentration of at least about 9%. In some aspects, the saccharide, e.g., sucrose or trehalose, is present in the composition at a concentration of at least about 10%.

In some aspects, the composition comprises at least about 1% sucrose. In some aspects, the composition comprises at least about 2% sucrose. In some aspects, the composition comprises at least about 2.5% sucrose. In some aspects, the composition comprises at least about 3% sucrose. In some aspects, the composition comprises at least about 4% sucrose. In some aspects, the composition comprises at least about 5% sucrose. In some aspects, the composition comprises at least about 6% sucrose. In some aspects, the composition comprises at least about 7% sucrose. In some aspects, the composition comprises at least about 8% sucrose. In some aspects, the composition comprises at least about 9% sucrose. In some aspects, the composition comprises at least about 10% sucrose.

In some aspects, the composition comprises at least about 1% trehalose. In some aspects, the composition comprises at least about 2% trehalose. In some aspects, the composition comprises at least about 3% trehalose. In some aspects, the composition comprises at least about 4% trehalose. In some aspects, the composition comprises at least about 5% trehalose. In some aspects, the composition comprises at least about 6% trehalose. In some aspects, the composition comprises at least about 7% trehalose. In some aspects, the composition comprises at least about 8% trehalose. In some aspects, the composition comprises at least about 9% trehalose. In some aspects, the composition comprises at least about 10% trehalose.

In some aspects, the saccharide, e.g., sucrose or trehalose, is present in the composition at a concentration from at least about 10 mg/ml to at least about 200 mg/ml, from at least about 100 mg/ml to at least about 200 mg/ml, from at least about 110 mg/ml to at least about 190 mg/ml, from at least about 120 mg/ml to at least about 180 mg/ml, from at least about 130 mg/ml to at least about 170 mg/ml, from at least about 140 mg/ml to at least about 160 mg/ml, or from at least about 140 mg/ml to at least about 150 mg/ml. In some aspects, the saccharide, e.g., sucrose or trehalose, is present in the composition at a concentration of at least about 146 mg/ml. In some aspects, the saccharide, e.g., sucrose or trehalose, is present in the composition at a concentration of at least about 145 mg/ml. In some aspects, the saccharide, e.g., sucrose or trehalose, is present in the composition at a concentration of at least about 140 mg/ml. In some aspects, the saccharide, e.g., sucrose or trehalose, is present in the composition at a concentration of at least about 135 mg/ml. In some aspects, the saccharide, e.g., sucrose or trehalose, is present in the composition at a concentration of at least about 150 mg/ml. In some aspects, the saccharide, e.g., sucrose or trehalose, is present in the composition at a concentration of at least about 155 mg/ml. In some aspects, the saccharide, e.g., sucrose or trehalose, is present in the composition at a concentration of at least about 160 mg/ml.

In some aspects, the composition comprises at least about 135 mg/ml sucrose. In some aspects, the composition comprises at least about 140 mg/ml sucrose. In some aspects, the composition comprises at least about 145 mg/ml sucrose. In some aspects, the composition comprises at least about 146 mg/ml sucrose. In some aspects, the composition comprises at least about 150 mg/ml sucrose. In some aspects, the composition comprises at least about 155 mg/ml sucrose. In some aspects, the composition comprises at least about 160 mg/ml sucrose.

In some aspects, the composition comprises at least about 135 mg/ml trehalose. In some aspects, the composition comprises at least about 140 mg/ml trehalose. In some aspects, the composition comprises at least about 145 mg/ml trehalose. In some aspects, the composition comprises at least about 146 mg/ml trehalose. In some aspects, the composition comprises at least about 150 mg/ml trehalose. In some aspects, the composition comprises at least about 155 mg/ml trehalose. In some aspects, the composition comprises at least about 160 mg/ml trehalose.

In certain aspects, the composition comprises at least about 2.5% sucrose, wherein the composition has improved stability compared to a similar composition comprising less than about 2% sucrose.

### II.F. Conductivity

In some aspects, the composition of the present disclosure has a conductivity between about 6 mS/cm+/−10% and about 10 mS/cm+/−10%. In some aspects, the conductivity is between 6 mS/cm+/−10% and about 7 mS/cm+/−10%, between about 7 mS/cm+/−10% and about 8 mS/cm+/−10%, between about 8 mS/cm+/−10% and about 9 mS/cm+/−10%, or between about 9 mS/cm+/−10% and about 10 mS/cm+/−10%. In some aspects, the conductivity is about 6 mS/cm+/−10%, about 7 mS/cm+/−10%, about 8 mS/cm+/−10%, about 9 mS/cm+/−10%, or about 10 mS/cm+/−10%.

In some aspects, the composition has a conductivity between about 6 mS/cm+/−10% and about 10 mS/cm+/−10%. In some aspects, the conductivity is about 6 mS/cm+/−10%. In some aspects, the conductivity is about 7 mS/cm+/−10%. In some aspects, the conductivity is about 8 mS/cm+/−10%. In some aspects, the conductivity is about 9 mS/cm+/−10%. In some aspects, the conductivity is about 10 mS/cm+/−10%. In some aspects, the conductivity is 7.23 mS/cm+/−10%. In some aspects, the conductivity is 8.8 mS/cm+/−10%.

### II.G. Anti-Oxidants

In some aspects, the composition of the present disclosure further comprises an anti-oxidant. In some aspects, the anti-oxidant comprises D-methionine, L-methionine. ascorbic acid, erythorbic acid, Na ascorbate, thioglycerol, cysteine, acetylcysteine, cystine, dithioerythreitol, glutathione, tocopherols, butylated hydroxyanisole (BHA), butylated hydroxytoluene (BHT), sodium bisulphate, sodium dithionite, A-Tocopherol, y-Tocopherol, propyl gallate, ascorbyl palmitate, sodium metabisulfite, thiourea, sodium thiosulfate, propyl gallate, Vitamin C, N-acetyl cysteine, selenium, and sodium thioglycolate In some aspects, the anti-oxidant is methionine. In other aspects, the anti-oxidant is D-methionine. In other aspects, the anti-oxidant is L-methionine.

In some aspects, the composition comprises a thiosulfate or a salt thereof. In some aspects, the thiosulphate or salt thereof comprises sodium thiosulphate.

In some aspects, a composition disclosed herein comprises an antireductant. In some aspects, the antireductant comprises EDTA, EGTA, CuSO4, S-adenosylmethionine, cysteine, or any combination thereof.

### II.H. Protease Inhibitors

In some aspects, a composition disclosed herein comprises a protease inhibitor. Proteins such as thioredoxin can reduce proteins with disulfide bonds. Addition of inhibitors such as EDTA, EGTA, and CuSO4 can reduce the activity, especially of metalloproteases such as hexokinase. EEGTA/EDTA inhibit by chelating divalent cations. Accordingly, in some aspects, the composition further comprises a protease inhibitor selected from EDTA, EGTA, CuSO4, and any combination thereof. In some aspects, the temperature is reduced in order to reduce the activity of a protease.

### II.I. Characteristics of Compositions

The compositions of the present disclosure have been formulated such that the EVs of the compositions are stable under fluctuating temperature conditions, e.g., when frozen and/or thawed and/or administrated to a subject. Without wishing to be bound, it is though that the combination of the presently disclosed saccharides, e.g., sucrose at about 5% w/v, with potassium phosphate and sodium phosphate, at the particular ratios presently disclosed, provides superior stability to the composition and the EVs contained therein. For example, in some aspects, the compositions of the present disclosure are capable of being stored for various lengths of time and at various temperatures, wherein the stability of the extracellular vesicle, e.g., exosome, is not reduced. Furthermore, in some aspects, the presently disclosed compositions can be formulated as a liquid in ambient temperature and then frozen by placing the compositions into a −80° C. freezer, and then thawed. Accordingly, in some aspects, the composition can be stored as a liquid, before freezing, the composition can be stored as a solid, while frozen, and the composition can be stored as a liquid, after thawing, without compromising the stability of the EV, as described below.

In some aspects, the composition can be stored as a liquid, before freezing. In some aspects, the composition can be stored as a liquid, before freezing, at temperatures between about 25° C. to about 1° C., wherein the stability of the EV, e.g., exosome, is not reduced. In some aspects, the composition can be stored as a liquid, before freezing, at about 25° C. to about 1° C., without compromising the stability of the EV, e.g., exosome.

In some aspects, the composition can be stored as a liquid, before freezing, for at least about 4 hours, at least about 10 hours, at least about 12 hours, at least about 15 hours, at least about 20 hours, at least about 24 hours. In some aspects, the composition is stored as a liquid, before freezing for about 4 hours to about 12 hours, about 5 hours to about 12 hours, about 6 hours to about 12 hours, about 4 hours to about 24 hours, about 6 hours to about 24 hours, about 12 hours to about 24 hours, or about 4 hours about 16 hours. In some aspects, the composition can be stored as a liquid, before freezing, for less than 36 hours, less than 30 days, less than 24 hours, less than 23 hours, less than 22 hours, less than 21 hours, less than 20 hours, less than 19 hours, less than 18 hours, less than 17 hours, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, or less than 4 hours.

In some aspects, the composition can be stored as a liquid at about 4° C. before freezing, for about one week. In some aspects, the composition can be stable for up to a week at 4° C. In some aspects, the composition can be stored as a liquid at about 4° C. before freezing, for about one week and then administered to a subject in need thereof.

In some aspects, the composition is capable of being stored as a frozen solid, for a length of time before being thawed. In some aspects, the composition can be stored as a solid, at zero and sub-zero temperatures, e.g., temperatures between about 0° C. and or −80° C., wherein the stability of the EV, e.g., exosome, is not reduced. In some aspects, the composition can be stored as a frozen solid, at temperatures between about 0° C. and or −80° C. In some aspects, the composition can be stored as a frozen solid, at temperatures between about 0° C. and −50° C. In some aspects, the composition can be stored as a frozen solid, at temperatures between about 0° C. and −20° C. In some aspects, the composition can be stored as a frozen solid, at temperatures between about 0° C. and −15° C. In some aspects, the composition can be stored for up to 6 months at −80° C. In some aspects, the composition can be stable for one year at −80° C. In some aspects, the composition can be stable for two years at −80° C.

The presently disclosed compositions can be stored as frozen solids for various lengths of time, and thereafter thawed, in preparation for administration to subjects in need thereof. In some aspects, the thawed liquids can be stored for various lengths and at various temperatures prior to administration, without compromising the stability of the EVs, e.g., exosomes. In some aspects, the composition is capable of being thawed and stored as a liquid, at a temperatures from about 1° C. to about 25° C., wherein the stability of the EV, e.g., exosome, is not reduced.

In some aspects, the composition can be stored as a thawed liquid, at about 1° C. In some aspects, the composition can be stored as a thawed liquid, at about 2° C. In some aspects, the composition can be stored as a thawed liquid, at about 3° C. In some aspects, the composition can be stored as a thawed liquid, at about 4° C. In some aspects, the composition can be stored as a thawed liquid, at about 5° C. In some aspects, the composition can be stored as a thawed liquid, at about 6° C. In some aspects, the composition can be stored as a thawed liquid, at about 7° C. In some aspects, the composition can be stored as a thawed liquid, at about 8° C. In some aspects, the composition can be stored as a thawed liquid, at about 9° C. In some aspects, the composition can be stored as a thawed liquid, at about 10° C. In some aspects, the composition can be stored as a thawed liquid, at about 11° C. In some aspects, the composition can be stored as a thawed liquid, at about 12° C. In some aspects, the composition can be stored as a thawed liquid, at about 13° C. In some aspects, the composition can be stored as a thawed liquid, at about 14° C. In some aspects, the composition can be stored as a thawed liquid, at about 15° C. In some aspects, the composition can be stored as a thawed liquid, at about 16° C. In some aspects, the composition can be stored as a thawed liquid, at about 17° C. In some aspects, the composition can be stored as a thawed liquid, at about 18° C. In some aspects, the composition can be stored as a thawed liquid, at about 19° C. In some aspects, the composition can be stored as a thawed liquid, at about 20° C. In some aspects, the composition can be stored as a thawed liquid, at about 21° C. In some aspects, the composition can be stored as a thawed liquid, at about 22° C. In some aspects, the composition can be stored as a thawed liquid, at about 23° C. In some aspects, the composition can be stored as a thawed liquid, at about 24° C. In some aspects, the composition can be stored as a thawed liquid, at about 25° C. In some aspects, the composition can be stored as a thawed liquid at 4° C., for about one week. In some aspects, the composition can be stable as a thawed liquid for up to a week at 4° C.

In some aspects, the composition can be stored, and then directly administered to a subject in need thereof. In some aspects, the composition can be stored for up to 24 hours at 25° C., and then directly administered to a subject in need thereof. In some aspects, the composition can be stored for up to 3 days at 4° C., and then directly administered to a subject in need thereof. In some aspects, the composition can be stored for up to 7 days at 4° C., and then directly administered to a subject in need thereof. In some aspects, the composition can be stored for up to 6 months at −80° C., thawed, and then directly administered to a subject in need thereof.

### II.J. Exemplary Compositions

In some aspects, the composition comprises:
a. an extracellular vesicle comprising an ASO, wherein the ASO comprises a contiguous nucleotide sequence of 10 to 30 nucleotides in length that is complementary to a nucleic acid sequence within a STAT6 transcript;
b. a sucrose at a concentration of about 5% w/v,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the composition is in a solution at a pH of 7.2; and
wherein the composition comprises an osmolarity of about 365 to about 425 mOsm/kg.

In some aspects, the composition comprises:
a. an extracellular vesicle comprising an ASO, wherein the ASO comprises a contiguous nucleotide sequence of 10 to 30 nucleotides in length that is complementary to a nucleic acid sequence within a STAT6 transcript;
b. a sucrose at a concentration of about 5% w/v,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
a. an extracellular vesicle comprising an ASO, wherein the ASO comprises a contiguous nucleotide sequence of 10 to 30 nucleotides in length that is complementary to a nucleic acid sequence within a STAT6 transcript;
b. a sucrose at a concentration of about 146 mM,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the composition is in a solution at a pH of 7.2; and
wherein the composition comprises an osmolarity of about 365 to about 425 mOsm/kg.

In some aspects, the composition comprises:
a. an extracellular vesicle comprising an ASO, wherein the ASO comprises a contiguous nucleotide sequence of 10 to 30 nucleotides in length that is complementary to a nucleic acid sequence within a STAT6 transcript;
b. a sucrose at a concentration of about 146 mM,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the composition is in a solution at a pH of 7.2; and wherein the composition comprises an osmolarity of about 395 mOsm/kg.

In some aspects, the composition comprises:
a. an extracellular vesicle comprising an ASO, wherein the ASO comprises a nucleic acid sequence selected from SEQ ID NOs: 91-193;
b. a sucrose at a concentration of about 5%,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
f. an extracellular vesicle comprising an ASO, wherein the ASO comprises a nucleic acid sequence selected from SEQ ID NOs: 91-193;
g. a sucrose at a concentration of about 146 mM,
h. sodium chloride at a concentration of about 100 mM;
i. a potassium phosphate monobasic at a concentration of about 5 mM;
j. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
a. an extracellular vesicle comprising an ASO, wherein the ASO comprises the nucleic acid sequence GAAAGGTTCCGTCGGGC (SEQ ID NO: 144);
b. a sucrose at a concentration of about 5%,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
f. an extracellular vesicle comprising an ASO, wherein the ASO comprises the nucleic acid sequence GAAAGGTTCCGTCGGGC (SEQ ID NO: 144);
g. a sucrose at a concentration of about 146 mM,
h. sodium chloride at a concentration of about 100 mM;
i. a potassium phosphate monobasic at a concentration of about 5 mM;
j. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
a. an extracellular vesicle comprising an ASO, wherein the ASO comprises the nucleic acid sequence CTGAGTCGCTGAAGCGG (SEQ ID NO: 145);
b. a sucrose at a concentration of about 5%,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
f. an extracellular vesicle comprising an ASO, wherein the ASO comprises the nucleic acid sequence CTGAGTCGCTGAAGCGG (SEQ ID NO: 145);
g. a sucrose at a concentration of about 146 mM,
h. sodium chloride at a concentration of about 100 mM;
i. a potassium phosphate monobasic at a concentration of about 5 mM;
j. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
a. an extracellular vesicle comprising an ASO, wherein the ASO comprises the nucleic acid sequence GCCCTTGTACTTTTGCATAG (SEQ ID NO: 193);
b. a sucrose at a concentration of about 5%,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
f. an extracellular vesicle comprising an ASO, wherein the ASO comprises the nucleic acid sequence GCCCTTGTACTTTTGCATAG (SEQ ID NO: 193);
g. a sucrose at a concentration of about 146 mM,
h. sodium chloride at a concentration of about 100 mM;
i. a potassium phosphate monobasic at a concentration of about 5 mM;
j. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
a. an extracellular vesicle comprising an ASO, wherein the ASO comprises the nucleic acid sequence GCAAGATCCCGGATTCGGTC (SEQ ID NO: 185);
b. a sucrose at a concentration of about 5%,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the composition comprises:
f. an extracellular vesicle comprising an ASO, wherein the ASO comprises the nucleic acid sequence GCAAGATCCCGGATTCGGTC (SEQ ID NO: 185);
g. a sucrose at a concentration of about 146 mM,
h. sodium chloride at a concentration of about 100 mM;
i. a potassium phosphate monobasic at a concentration of about 5 mM;
j. a sodium phosphate dibasic heptahydrate at a concentration of about 15 mM;
wherein the composition is in a solution at a pH of 7.2; and
wherein the composition comprises an osmolarity of about 395 mOsm/kg.

In some aspects, the ASO comprises a nucleic acid sequence selected from SEQ ID NOs: 91-193. In some aspects, the ASO comprises the nucleic acid sequence GAAAGGTTCCGTCGGGC (SEQ ID NO: 144). In some aspects, the ASO comprises the nucleic acid sequence CTGAGTCGCTGAAGCGG (SEQ ID NO: 145). In some aspects, the ASO comprises the nucleic acid sequence GCCCTTGTACTTTTGCATAG (SEQ ID NO: 193). In some aspects, the ASO comprises the nucleic acid sequence GCAAGATCCCGGATTCGGTC (SEQ ID NO: 185).

In certain aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose;
(c) Sodium chloride;
(d) Potassium phosphate monobasic at a concentration of about 5 mM;
(e) Sodium phosphate dibasic at a concentration of about 15 mM,
(f) wherein the pH of the composition is about 7.2;
wherein the sucrose is at a concentration selected from about 73 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM;
wherein the sodium chloride is at a concentration selected from about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, and about 150 mM; and
wherein the composition comprises an osmolarity of about 395 mOsm/kg.

In certain aspects, the composition comprises:
(a) Extracellular vesicles comprising an ASO;
(b) Sucrose;
(c) Sodium chloride;
(d) Potassium phosphate monobasic at a concentration of about 5 mM;
(e) Sodium phosphate dibasic at a concentration of about 15 mM,
(f) wherein the pH of the composition is about 7.2;
wherein the sucrose is at a concentration selected from about 73 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM;
wherein the sodium chloride is at a concentration selected from about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, and about 150 mM; and
wherein the composition comprises an osmolarity of about 395 mOsm/kg.

In some aspects, wherein the ASO comprises a nucleic acid sequence selected from SEQ ID NOs: 91-193.

In some aspects, the composition comprises:
(a) Extracellular vesicles comprising an ASO;
(b) Sucrose;
(c) Sodium chloride;
(d) Potassium phosphate monobasic at a concentration of about 5 mM;
(e) Sodium phosphate dibasic at a concentration of about 15 mM,
(f) wherein the pH of the composition is about 7.2;
wherein the sucrose is at a concentration selected from about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, and about 5.0%;
wherein the sodium chloride is at a concentration selected from about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, and about 150 mM; and
wherein the composition comprises an osmolarity of about 395 mOsm/kg.

In some aspects, wherein the ASO comprises a nucleic acid sequence selected from SEQ ID NOs: 91-193.

In certain aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 73 mM to about 146 mM;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 2.5% to about 5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In certain aspects, the composition comprises:
(a) Extracellular vesicles comprising an ASO, wherein the ASO comprises a nucleic acid sequence selected from SEQ ID NOs: 91-193;
(b) Sucrose at a concentration of about 73 mM to about 146 mM;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 2.5% to about 5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 2.5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the ASO comprises a nucleic acid sequence selected from SEQ ID NOs: 91-93.

In certain aspects, the composition comprises:
(a) Extracellular vesicles comprising an ASO, wherein the ASO comprises a nucleic acid sequence selected from SEQ ID NOs: 91-193;
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and wherein the composition comprises an osmolarity of about 395 mOsm/kg.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 4.5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 4%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 3.5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 3%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 2.5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In certain aspects, the composition comprises:
(a) Extracellular vesicles comprising an ASO, wherein the ASO comprises the nucleic acid sequence GAAAGGTTCCGTCGGGC (SEQ ID NO: 144);
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises the nucleic acid sequence GAAAGGTTCCGTCGGGC (SEQ ID NO: 144);
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In certain aspects, the composition comprises:
(a) Extracellular vesicles comprising an ASO, wherein the ASO comprises the nucleic acid sequence CTGAGTCGCTGAAGCGG (SEQ ID NO: 145);
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises the nucleic acid sequence CTGAGTCGCTGAAGCGG (SEQ ID NO: 145);
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In certain aspects, the composition comprises:
(a) Extracellular vesicles comprising an ASO, wherein the ASO comprises the nucleic acid sequence GCCCTTGTACTTTTGCATAG (SEQ ID NO: 193);
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises the nucleic acid sequence GCCCTTGTACTTTTGCATAG (SEQ ID NO: 193);
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In certain aspects, the composition comprises:
(a) Extracellular vesicles comprising an ASO, wherein the ASO comprises the nucleic acid sequence GCAAGATCCCGGATTCGGTC (SEQ ID NO: 185);
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition comprises:
(a) Extracellular vesicles comprising an ASO, wherein the ASO comprises the nucleic acid sequence GCAAGATCCCGGATTCGGTC (SEQ ID NO: 185);
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2; and
wherein the composition comprises an osmolarity of about 395 mOsm/kg.

In some aspects, the composition is lyophilized.

### III. Extracellular Vesicles, e.g., Exosomes

Disclosed herein are modified EVs, e.g., exosomes, capable of regulating the immune system of a subject. The EVs, e.g., exosomes, useful in the present disclosure have been engineered to produce at least one exogenous biologically active moiety. In some aspects, an EV (e.g., exosome) comprises two exogenous biologically active moieties. In some aspects, an EV (e.g., exosome) comprises three exogenous biologically active moieties. In other aspects, an EV (e.g., exosome) comprises four exogenous biologically active moieties. In further aspects, an EV (e.g., exosome) comprises five or more exogenous biologically active moieties. In some aspects, an EV (e.g., exosome) comprises 6, 7, 8, 9, 10, 11, 12, 13, 14, 15, 16, 17, 18, 19, 20 or more exogenous biologically active moieties.

As described supra, EVs, e.g., exosomes, described herein are extracellular vesicles with a diameter between about 20-300 nm. In certain aspects, an EV, e.g., exosome, of the present disclosure has a diameter between about 20-290 nm, 20-280 nm, 20-270 nm, 20-260 nm, 20-250 nm, 20-240 nm, 20-230 nm, 20-220 nm, 20-210 nm, 20-200 nm, 20-190 nm, 20-180 nm, 20-170 nm, 20-160 nm, 20-150 nm, 20-140 nm, 20-130 nm, 20-120 nm, 20-110 nm, 20-100 nm, 20-90 nm, 20-80 nm, 20-70 nm, 20-60 nm, 20-50 nm, 20-40 nm, 20-30 nm, 30-300 nm, 30-290 nm, 30-280 nm, 30-270 nm, 30-260 nm, 30-250 nm, 30-240 nm, 30-230 nm, 30-220 nm, 30-210 nm, 30-200 nm, 30-190 nm, 30-180 nm, 30-170 nm, 30-160 nm, 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-300 nm, 40-290 nm, 40-280 nm, 40-270 nm, 40-260 nm, 40-250 nm, 40-240 nm, 40-230 nm, 40-220 nm, 40-210 nm, 40-200 nm, 40-190 nm, 40-180 nm, 40-170 nm, 40-160 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-300 nm, 50-290 nm, 50-280 nm, 50-270 nm, 50-260 nm, 50-250 nm, 50-240 nm, 50-230 nm, 50-220 nm, 50-210 nm, 50-200 nm, 50-190 nm, 50-180 nm, 50-170 nm, 50-160 nm, 50-150 nm, 50-140 nm, 50-130 nm, 50-120 nm, 50-110 nm, 50-100 nm, 50-90 nm, 50-80 nm, 50-70 nm, 50-60 nm, 60-300 nm, 60-290 nm, 60-280 nm, 60-270 nm, 60-260 nm, 60-250 nm, 60-240 nm, 60-230 nm, 60-220 nm, 60-210 nm, 60-200 nm, 60-190 nm, 60-180 nm, 60-170 nm, 60-160 nm, 60-150 nm, 60-140 nm, 60-130 nm, 60-120 nm, 60-110 nm, 60-100 nm, 60-90 nm, 60-80 nm, 60-70 nm, 70-300 nm, 70-290 nm, 70-280 nm, 70-270 nm, 70-260 nm, 70-250 nm, 70-240 nm, 70-230 nm, 70-220 nm, 70-210 nm, 70-200 nm, 70-190 nm, 70-180 nm, 70-170 nm, 70-160 nm, 70-150 nm, 70-140 nm, 70-130 nm, 70-120 nm, 70-110 nm, 70-100 nm, 70-90 nm, 70-80 nm, 80-300 nm, 80-290 nm, 80-280 nm, 80-270 nm, 80-260 nm, 80-250 nm, 80-240 nm, 80-230 nm, 80-220 nm, 80-210 nm, 80-200 nm, 80-190 nm, 80-180 nm, 80-170 nm, 80-160 nm, 80-150 nm, 80-140 nm, 80-130 nm, 80-120 nm, 80-110 nm, 80-100 nm, 80-90 nm, 90-300 nm, 90-290 nm, 90-280 nm, 90-270 nm, 90-260 nm, 90-250 nm, 90-240 nm, 90-230 nm, 90-220 nm, 90-210 nm, 90-200 nm, 90-190 nm, 90-180 nm, 90-170 nm, 90-160 nm, 90-150 nm, 90-140 nm, 90-130 nm, 90-120 nm, 90-110 nm, 90-100 nm, 100-300 nm, 110-290 nm, 120-280 nm, 130-270 nm, 140-260 nm, 150-250 nm, 160-240 nm, 170-230 nm, 180-220 nm, or 190-210 nm. The size of the EV, e.g., exosome, described herein can be measured according to methods described, infra.

In some aspects, an EV, e.g., exosome, of the present disclosure comprises a bi-lipid membrane (“EV, e.g., exosome, membrane”), comprising an interior surface and an exterior surface. In certain aspects, the interior surface faces the inner core (i.e., lumen) of the EV, e.g., exosome. In certain aspects, the exterior surface can be in contact with the endosome, the multivesicular bodies, or the membrane/cytoplasm of a producer cell or a target cell

In some aspects, the EV, e.g., exosome, membrane comprises lipids and fatty acids. In some aspects, the EV, e.g., exosome, membrane comprises phospholipids, glycolipids, fatty acids, sphingolipids, phosphoglycerides, sterols, cholesterols, and phosphatidylserines.

In some aspects, the EV, e.g., exosome, membrane comprises an inner leaflet and an outer leaflet. The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine.

In some aspects, the EV, e.g., exosome, membrane comprises one or more polysaccharide, such as glycan.

In some aspects, the EV, e.g., exosome, membrane further comprises one or more scaffold moieties, which are capable of anchoring the multiple exogenous biologically active moieties to the EV, e.g., exosome, (e.g., either on the luminal surface or on the exterior surface). In some aspects, the scaffold moieties anchor or link at least one of the multiple exogenous biologically active moieties to the EV. In some aspects, the scaffold moieties anchor or link each of the multiple (e.g., at least two) exogenous biologically active moieties to the EV. In certain aspects, scaffold moieties are polypeptides (“exosome proteins”). In other aspects, scaffold moieties are non-polypeptide moieties. In some aspects, exosome proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., exosome protein) comprises Scaffold X. In further aspects, a scaffold moiety (e.g., exosome protein) comprises more than one Scaffold X moiety.

In some aspects, an EV, e.g., exosome, disclosed herein is capable of delivering one or more payload (e.g., a biologically active moiety) to a target. Accordingly, in certain aspects, an EV (e.g., exosome) comprises one, two, three, four, five or more different payloads. The payload is an agent that acts on a target (e.g., a target cell) that is contacted with the EV. Contacting can occur in vitro or in a subject. Non-limiting examples of payloads that can be introduced into an EV include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, or siRNA), RNA binding proteins such as MS2, amino acids (e.g., amino acids comprising a detectable moiety or a toxin that disrupt translation), polypeptides (e.g., enzymes), lipids, carbohydrates, and small molecules (e.g., small molecule drugs and toxins). In some aspects, a payload comprises an exogenous biologically active moiety (e.g., those disclosed herein).

### III.A. Scaffold Moieties, e.g., Scaffold X or Scaffold Y

In some aspects, EVs, e.g., exosomes, of the present disclosure comprise a membrane modified in its composition. For example, their membrane compositions can be modified by changing the protein, lipid, or glycan content of the membrane.

In some aspects, the surface-engineered EVs, e.g., exosomes, are generated by chemical and/or physical methods, such as PEG-induced fusion and/or ultrasonic fusion. In other aspects, the surface-engineered EVs, e.g., exosomes, are generated by genetic engineering. EVs, e.g., exosomes, produced from a genetically-modified producer cell or a progeny of the genetically-modified cell can contain modified membrane compositions. In some aspects, surface-engineered EVs, e.g., exosomes, have scaffold moiety (e.g., exosome protein, e.g., Scaffold X) at a higher or lower density (e.g., higher number) or include a variant or a fragment of the scaffold moiety.

For example, surface (e.g., Scaffold X)-engineered EVs, can be produced from a cell (e.g., HEK293 cells) transformed with an exogenous sequence encoding a scaffold moiety (e.g., exosome proteins, e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, scaffold moiety modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Non-limiting examples of Scaffold moieties include: prostaglandin F2 receptor negative regulator (PTGFRN); basigin (BSG); immunoglobulin superfamily member 2 (IGSF2); immunoglobulin superfamily member 3 (IGSF3); immunoglobulin superfamily member 8 (IGSF8); integrin beta-1 (ITGB1); integrin alpha-4 (ITGA4); 4F2 cell-surface antigen heavy chain (SLC3A2); and a class of ATP transporter proteins (ATP1A1, ATP1A2, ATP1A3, ATP1A4, ATP1B3, ATP2B1, ATP2B2, ATP2B3, ATP2B). In certain aspects, Scaffold moieties is a whole protein. In other aspects, Scaffold moieties is a protein fragment (e.g., functional fragment).

In other aspects, the scaffold moiety useful for the present disclose, a first scaffold moiety, a second scaffold moiety, and/or a third scaffold moiety, includes a conventional exosome protein, including, but not limiting, tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin and fragments thereof, peptides that have affinity to any of these proteins or fragments thereof, or any combination thereof.

In some aspects, the surface (e.g., Scaffold X)-engineered EVs described herein demonstrate superior characteristics compared to EVs known in the art. For example, surface (e.g., Scaffold X)-engineered EVs contain modified proteins more highly enriched on their surface than naturally occurring EVs or the EVs produced using conventional exosome proteins. Moreover, the surface (e.g., Scaffold X)-engineered EVs of the present disclosure can have greater, more specific, or more controlled biological activity compared to naturally occurring EVs or the EVs produced using conventional exosome proteins.

In some aspects the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at Table 2 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 9. In other aspects, the Scaffold X comprises the amino acid sequence of SEQ ID NO: 9, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 9 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 9.

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 6, 7, 8, 10, 11, or 12. In other aspects, the Scaffold X comprises the amino acid sequence of SEQ ID NO: 6, 7, 8, 10, 11, or 12, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 6, 7, 8, 10, 11, or 12 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 6, 7, 8, 10, 11, or 12.

TABLE 2
Exemplary Scaffold X Protein Sequences

| SEQ | | |
|---|---|---|
| ID | | |
| NO. | Protein | Sequence |
| 1. | Full | MGRLASRPLLLALLSLALCRGRVVRVPTATLVRVVGTELVIPCNVSDYDGPSEQNFDWSFSSL |
| | length | GSSFVELASTWEVGFPAQLYQERLQRGEILLRRTANDAVELHIKNVQPSDQGHYKCSTPSTDA |
| | PTGFRN | TVQGNYEDTVQVKVLADSLHVGPSARPPPSLSLREGEPFELRCTAASASPLHTHLALLWEVHR |
| | Protein | GPARRSVLALTHEGRFHPGLGYEQRYHSGDVRLDTVGSDAYRLSVSRALSADQGSYRCIVSEW |
| | | IAEQGNWQEIQEKAVEVATVVIQPSVLRAAVPKNVSVAEGKELDLTCNITTDRADDVRPEVTW |
| | | SFSRMPDSTLPGSRVLARLDRDSLVHSSPHVALSHVDARSYHLLVRDVSKENSGYYYCHVSLW |
| | | APGHNRSWHKVAEAVSSPAGVGVTWLEPDYQVYLNASKVPGFADDPTELACRVVDTKSGEANV |
| | | RFTVSWYYRMNRRSDNVVTSELLAVMDGDWTLKYGERSKQRAQDGDFIFSKEHTDTFNFRIQR |
| | | TTEEDRGNYYCVVSAWTKQRNNSWVKSKDVFSKPVNIFWALEDSVLVVKARQPKPFFAAGNTF |
| | | EMTCKVSSKNIKSPRYSVLIMAEKPVGDLSSPNETKYIISLDQDSVVKLENWTDASRVDGVVL |
| | | EKVQEDEFRYRMYQTQVSDAGLYRCMVTAWSPVRGSLWREAATSLSNPIEIDFQTSGPIFNAS |
| | | VHSDTPSVIRGDLIKLFCIITVEGAALDPDDMAFDVSWFAVHSFGLDKAPVLLSSLDRKGIVT |
| | | TSRRDWKSDLSLERVSVLEFLLQVHGSEDQDFGNYYCSVTPWVKSPTGSWQKEAEIHSKPVFI |
| | | TVKMDVLNAFKYPLLIGVGLSTVIGLLSCLIGYCSSHWCCKKEVQETRRERRRLMSMEMD |
| 6 | PTGFRN | PSARPPPSLS LREGEPFELR CTAASASPLH THLALLWEVH RGPARRSVLA |
| | Protein | LTHEGRFHPG LGYEQRYHSG DVRLDTVGSD AYRLSVSRAL SADQGSYRCI |
| | Fragment | VSEWIAEQGN WQEIQEKAVE VATVVIQPSV LRAAVPKNVS VAEGKELDLT |
| | #1 | CNITTDRADD VRPEVTWSFS RMPDSTLPGS RVLARLDRDS LVHSSPHVAL |
| | | SHVDARSYHL LVRDVSKENS GYYYCHVSLW APGHNRSWHK VAEAVSSPAG |
| | | VGVTWLEPDY QVYLNASKVP GFADDPTELA CRVVDTKSGE ANVRFTVSWY |
| | | YRMNRRSDNV VTSELLAVMD GDWTLKYGER SKQRAQDGDF IFSKEHTDTF |
| | | NFRIQRTTEE DRGNYYCVVS AWTKQRNNSW VKSKDVFSKP VNIFWALEDS |
| | | VLVVKARQPK PFFAAGNTFE MTCKVSSKNI KSPRYSVLIM AEKPVGDLSS |
| | | PNETKYIISL DQDSVVKLEN WTDASRVDGV VLEKVQEDEF RYRMYQTQVS |
| | | DAGLYRCMVT AWSPVRGSLW REAATSLSNP IEIDFQTSGP IFNASVHSDT |
| | | PSVIRGDLIK LFCIITVEGA ALDPDDMAFD VSWFAVHSFG LDKAPVLLSS |
| | | LDRKGIVTTS RRDWKSDLSL ERVSVLEFLL QVHGSEDQDF GNYYCSVTPW |
| | | VKSPTGSWQK EAEIHSKPVF ITVKMDVLNA FKYPLLIGVG LSTVIGLLSC |
| | | LIGYCSSHWC CKKEVQETRR ERRRLMSMEM D |
| 7 | PTGFRN | VATVVIQPSV LRAAVPKNVS VAEGKELDLT CNITTDRADD VRPEVTWSFS |
| | Protein | RMPDSTLPGS RVLARLDRDS LVHSSPHVAL SHVDARSYHL LVRDVSKENS |
| | Fragment | GYYYCHVSLW APGHNRSWHK VAEAVSSPAG VGVTWLEPDY QVYLNASKVP |
| | #2 | GFADDPTELA CRVVDTKSGE ANVRFTVSWY YRMNRRSDNV VTSELLAVMD |
| | | GDWTLKYGER SKQRAQDGDF IFSKEHTDTF NFRIQRTTEE DRGNYYCVVS |
| | | AWTKQRNNSW VKSKDVFSKP VNIFWALEDS VLVVKARQPK PFFAAGNTFE |
| | | MTCKVSSKNI KSPRYSVLIM AEKPVGDLSS PNETKYIISL DQDSVVKLEN |
| | | WTDASRVDGV VLEKVQEDEF RYRMYQTQVS DAGLYRCMVT AWSPVRGSLW |
| | | REAATSLSNP IEIDFQTSGP IFNASVHSDT PSVIRGDLIK LFCIITVEGA |
| | | ALDPDDMAFD VSWFAVHSFG LDKAPVLLSS LDRKGIVTTS RRDWKSDLSL |
| | | ERVSVLEFLL QVHGSEDQDF GNYYCSVTPW VKSPTGSWQK EAEIHSKPVF |
| | | ITVKMDVLNA FKYPLLIGVG LSTVIGLLSC LIGYCSSHWC CKKEVQETRR |
| | | ERRRLMSMEM D |
| 8 | PTGFRN | SPAGVGVTWL EPDYQVYLNA SKVPGFADDP TELACRVVDT KSGEANVRFT |
| | Protein | VSWYYRMNRR SDNVVTSELL AVMDGDWTLK YGERSKQRAQ DGDFIFSKEH |
| | Fragment | TDTFNFRIQR TTEEDRGNYY CVVSAWTKQR NNSWVKSKDV FSKPVNIFWA |
| | #3 | LEDSVLVVKA RQPKPFFAAG NTFEMTCKVS SKNIKSPRYS VLIMAEKPVG |
| | | DLSSPNETKY IISLDQDSVV KLENWTDASR VDGVVLEKVQ EDEFRYRMYQ |
| | | TQVSDAGLYR CMVTAWSPVR GSLWREAATS LSNPIEIDFQ TSGPIFNASV |
| | | HSDTPSVIRG DLIKLFCIIT VEGAALDPDD MAFDVSWFAV HSFGLDKAPV |
| | | LLSSLDRKGI VTTSRRDWKS DLSLERVSVL EFLLQVHGSE DQDFGNYYCS |
| | | VTPWVKSPTG SWQKEAEIHS KPVFITVKMD VLNAFKYPLL IGVGLSTVIG |
| | | LLSCLIGYCS SHWCCKKEVQ ETRRERRRLM SMEMD |
| 10 | PTGFRN | KPVNIFWALE DSVLVVKARQ PKPFFAAGNT FEMTCKVSSK NIKSPRYSVL |
| | Protein | IMAEKPVGDL SSPNETKYII SLDQDSVVKL ENWTDASRVD GVVLEKVQED |
| | Fragment | EFRYRMYQTQ VSDAGLYRCM VTAWSPVRGS LWREAATSLS NPIEIDFQTS |
| | #4 | GPIFNASVHS DTPSVIRGDL IKLFCIITVE GAALDPDDMA FDVSWFAVHS |
| | | FGLDKAPVLL SSLDRKGIVT TSRRDWKSDL SLERVSVLEF LLQVHGSEDQ |
| | | DFGNYYCSVT PWVKSPTGSW QKEAEIHSKP VFITVKMDVL NAFKYPLLIG |
| | | VGLSTVIGLL SCLIGYCSSH WCCKKEVQET RRERRRLMSM EMD |
| 11 | PTGFRN | VRGSLWREAA TSLSNPIEID FQTSGPIFNA SVHSDTPSVI RGDLIKLFCI |
| | Protein | ITVEGAALDP DDMAFDVSWF AVHSFGLDKA PVLLSSLDRK GIVTTSRRDW |
| | Fragment | KSDLSLERVS VLEFLLQVHG SEDQDFGNYY CSVTPWVKSP TGSWQKEAEI |
| | #5 | HSKPVFITVK MDVLNAFKYP LLIGVGLSTV IGLLSCLIGY CSSHWCCKKE |
| | | VQETRRERRR LMSMEMD |
| 12 | PTGFRN | SKPVFITVKM DVLNAFKYPL LIGVGLSTVI GLLSCLIGYC SSHWCCKKEV |
| | Protein | QETRRERRRL MSMEMD |
| | Fragment | |
| | #6 | |
| 13 | PTGFRN | MGRLASRPLL LALLSLALCR G |
| | Protein - | |
| | Signal | |
| | Peptide | |
| 9 | PTGFRN | GPIFNASVHS DTPSVIRGDL IKLFCIITVE GAALDPDDMA FDVSWFAVHS |
| | Protein | FGLDKAPVLL SSLDRKGIVT TSRRDWKSDL SLERVSVLEF LLQVHGSEDQ |
| | Fragment | DFGNYYCSVT PWVKSPTGSW QKEAEIHSKP VFITVKMDVL NAFKYPLLIG |
| | #7 | VGLSTVIGLL SCLIGYCSSH WCCKKEVQET RRERRRLMSM EMD |

Non-limiting examples of other Scaffold X proteins can be found at U.S. Pat. No. 10,195,290 B1 and U.S. Pat. No. 10,561,740 B2, each of which is incorporated by reference in its entirety.

In some aspects, Scaffold X can be used to link any moiety to the luminal surface and on the exterior surface of the EV, e.g., exosome, at the same time. For example, the PTGFRN polypeptide can be used to link an antigen, an adjuvant, and/or an immune modulator inside the lumen (e.g., on the luminal surface) in addition to the exterior surface of the EV, e.g., exosome. Therefore, in certain aspects, Scaffold X can be used for dual purposes, e.g., an antigen on the luminal surface and an adjuvant or immune modulator on the exterior surface of the EV, e.g., exosome, an antigen on the exterior surface of the EV, e.g., exosome, and the adjuvant or immune modulator on the luminal surface, an adjuvant on the luminal surface and an immune modulator on the exterior surface of the EV, e.g., exosome, or an immune modulator on the luminal surface and an adjuvant on the exterior surface of the EV, e.g., exosome.

In some aspects, the scaffold protein comprises a Scaffold Y. Non-limiting examples of Scaffold Y proteins that can be used in the compositions and methods disclosed herein include those Scaffold Y proteins disclosed, for example, in International Publication No. WO/2019/099942 or WO 2020/101740, each of which is incorporated herein by reference in its entirety. In some aspects, the Scaffold Y protein is selected from myristoylated alanine rich Protein Kinase C substrate (“the MARCKS protein”); myristoylated alanine rich Protein Kinase C substrate like 1 (“the MARCKSL1 protein”); brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety on the luminal surface of the EVs, e.g., exosomes). In some aspects, a Scaffold Y can anchor a moiety to the lumen of the EVs, e.g., exosomes.

### III.B. Anchoring Moieties

In some aspects, an ASO disclosed herein or one or more payloads disclosed herein can be linked to an anchoring moiety. In some aspects, anchoring moieties that can be used to link a payload to the exterior surface and/or luminal surface of the EV (e.g., exosome) comprises: a sterol (e.g., cholesterol), GM1, a lipid (e.g., fatty acid), a vitamin, a small molecule, a peptide, or a combination thereof.

In some aspects, the anchoring moiety is a lipid. A lipid anchoring moiety can be any lipid known in the art, e.g., palmitic acid or glycosylphosphatidylinositols. In some aspects, the lipid is a fatty acid, phosphatide, phospholipid (e.g., phosphatidyl choline, phosphatidyl serine, or phosphatidyl ethanolamine), or analogue thereof (e.g. phophatidylcholine, lecithin, phosphatidylethanolamine, cephalin, or phosphatidylserine or analogue or portion thereof, such as a partially hydrolyzed portion thereof).

Generally, anchoring moieties are chemically attached. However, an anchoring moiety can be attached to a payload enzymatically.

Some types of membrane anchors that can be used to practice the methods of the present disclosure are presented in the following table.

| Modification | Modifying Group |
|---|---|
| S-Palmitoylation | 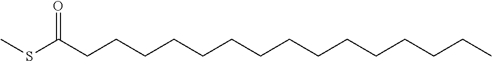 [SMILES: CCCCCCCCCCCCCCCC(=O)SC] |
| N-Palmitoylation | 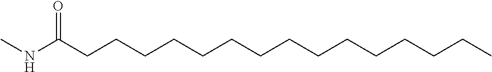 [SMILES: CCCCCCCCCCCCCCCC(=O)NC] |
| N-Myristoylalion | 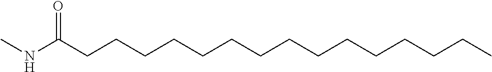 [SMILES: CCCCCCCCCCCCCCCC(=O)NC] |
| O-Acylation | 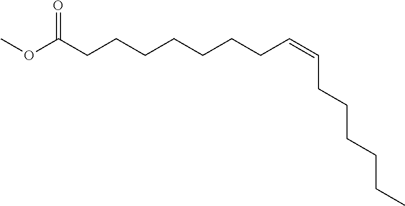 [SMILES: CCCCCC/C=C\CCCCCCCC(=O)OC] |
| Fernesylation | 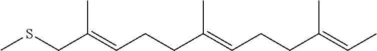 [SMILES: C/C=C(\C)CC/C=C(\C)CC/C=C(\C)CSC] |
| Geranylgeranylation | 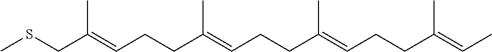 [SMILES: C/C=C(\C)CC/C=C(\C)CC/C=C(\C)CC/C=C(\C)CSC] |
| Cholesterol | 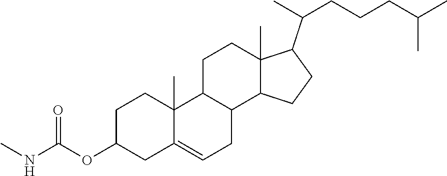 [SMILES: CNC(=O)OC1CCC2(C)C(=CCC3C2CCC2(C)C(C(C)CCCC(C)C)CCC32)C1] |

In some aspects, an anchoring moiety of the present disclosure can comprise two or more types of anchoring moieties disclosed herein. For example, in some aspects, an anchoring moiety can comprise two lipids, e.g., a phospholipids and a fatty acid, or two phospholipids, or two fatty acids, or a lipid and a vitamin, or cholesterol and a vitamin.

In some aspects, the anchoring moiety useful for the present disclosure comprises a sterol, steroid, hopanoid, hydroxysteroid, secosteroid, or analog thereof with lipophilic properties. In some aspects, the anchoring moiety comprises a sterol, such as a phytosterol, mycosterol, or zoosterol. Exemplary zoosterols include cholesterol and 24S-hydroxycholesterol; exemplary phytosterols include ergosterol (mycosterol), campesterol, sitosterol, and stigmasterol. In some aspects, the sterol is selected from ergosterol, 7-dehydrocholesterol, cholesterol, 24S-hydroxycholesterol, lanosterol, cycloartenol, fucosterol, saringosterol, campesterol, P-sitosterol, sitostanol, coprostanol, avenasterol, or stigmasterol. Sterols may be found either as free sterols, acylated (sterol esters), alkylated (steryl alkyl ethers), sulfated (sterol sulfate), or linked to a glycoside moiety (steryl glycosides), which can be itself acylated (acylated sterol glycosides). In some aspects, the anchoring moiety is a cholesterol.

In some aspects, the anchoring moiety comprises a steroid. In some aspects, the steroid is selected from dihydrotestosterone, uvaol, hecigenin, diosgenin, progesterone, or cortisol.

In some aspects, the anchoring moiety is a fatty acid. In some aspects, the fatty acid is a short-chain, medium-chain, or long-chain fatty acid. In some aspects, the fatty acid is a saturated fatty acid. In some aspects, the fatty acid is an unsaturated fatty acid. In some aspects, the fatty acid is a monounsaturated fatty acid. In some aspects, the fatty acid is a polyunsaturated fatty acid, such as an omega-3 or omega-6 fatty acid.

In some aspects, the anchoring moiety comprises a phospholipid. Phospholipids are a class of lipids that are a major component of all cell membranes. They can form lipid bilayers because of their amphiphilic characteristic. The structure of the phospholipid molecule generally consists of two hydrophobic fatty acid “tails” and a hydrophilic “head” consisting of a phosphate group. For example, a phospholipid can be a lipid according to the following formula:

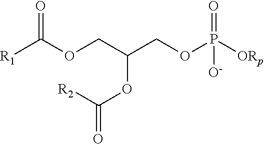

in which Rₚ represents a phospholipid moiety and R₁ and R₂ represent fatty acid moieties with or without unsaturation that may be the same or different.

In some aspects, a payload is linked to an anchoring moiety disclosed herein via a linker combination, which can comprise any combination of cleavable and/or non-cleavable linkers. Not to be bound by any one theory, one of the functions of a linker combination is to provide the optimal spacing between the anchoring moiety and the payload.

### III.C. Linkers

As described supra, extracellular vesicles (EVs) of the present disclosure (e.g., exosomes and nanovesicles) can comprises one or more linkers that link one or more exogenous biologically active moieties disclosed herein to the EVs (e.g., to the exterior surface or on the luminal surface). In some aspects, the one or more exogenous biologically active moieties are linked to the EVs directly or via one or more scaffold moieties (e.g., Scaffold X). For example, in certain aspects, one or more exogenous biologically active moieties are linked to the exterior surface of an exosome via Scaffold X. In further aspects, one or more exogenous biologically active moieties are linked to the luminal surface of an exosome via Scaffold X. The linker can be any chemical moiety known in the art.

As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

Linkers can be susceptible to cleavage (“cleavable linker”) thereby facilitating release of the exogenous biologically active moiety.

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the ASO is associated with the EV, e.g., exosome, by way of a linker. In some aspects, the linker comprises acrylic phosphoramidite (e.g., ACRYDITE™) adenylation, azide (NHS Ester), digoxigenin (NHS Ester), cholesterol-TEG, I-LINKER™, an amino modifier (e.g., amino modifier C6, amino modifier C12, amino modifier C6 dT, or Uni-Link™ amino modifier), alkyne, 5′ Hexynyl, 5-Octadiynyl dU, biotinylation (e.g., biotin, biotin (Azide), biotin dT, biotin-TEG, dual biotin, PC biotin, or desthiobiotin), thiol modification (thiol modifier C3 S—S, dithiol or thiol modifier C6 S—S), or any combination thereof.

In some aspects, the linker comprises a terpene such as nerolidol, farnesol, limonene, linalool, geraniol, carvone, fenchone, or menthol; a lipid such as palmitic acid or myristic acid; cholesterol; oleyl; retinyl; cholesteryl residues; cholic acid; adamantane acetic acid; 1-pyrene butyric acid; dihydrotestosterone; 1,3-Bis-O(hexadecyl)glycerol; geranyloxyhexyl group; hexadecylglycerol; borneol; 1,3-propanediol; heptadecyl group; 03-(oleoyl)lithocholic acid; 03-(oleoyl)cholenic acid; dimethoxytrityl; phenoxazine, a maleimide moiety, a glucorinidase type, a CL2A-SN38 type, folic acid; a carbohydrate; vitamin A; vitamin E; vitamin K, or any combination thereof. In certain aspects, the ASO comprises a cholesterol tag, and the cholesterol tag associates with the membrane of the EV, e.g., exosome. In some aspects, the linker comprises a non-cleavable linker.

In some aspects, the linker comprises tetraethylene glycol (TEG), hexaethylene glycol (HEG), polyethylene glycol (PEG), succinimide, or any combination thereof. In some aspects, the linker comprises a spacer unit to link the biologically active molecule to the linker.

In some aspects, one or more linkers comprise smaller units (e.g., HEG, TEG, glycerol, C2 to C12 alkyl, and the like) linked together. In one aspect, the linkage is an ester linkage (e.g., phosphodiester or phosphorothioate ester) or other linkage.

In some aspects, the linker comprises a polyethylene glycol (PEG) characterized by a formula R³—(O—CH₂—CH₂)ₙ— or R³-(0-CH₂—CH₂)ₙ—O— with R³ being hydrogen, methyl or ethyl and n having a value from 2 to 200. In some aspects, the linker comprises a spacer, wherein the spacer is PEG.

In some aspects, the PEG linker is an oligo-ethylene glycol, e.g., diethylene glycol, triethylene glycol, tetra ethylene glycol (TEG), pentaethylene glycol, or a hexaethylene glycol (HEG) linker.

### IV. Methods of Treatment

Administration of the presently disclosed pharmaceutical compositions for treating a plurality of diseases or conditions where administration of EVs are of beneficial effect to a subject, is further contemplated. In some aspects, the methods of treating a disease or a condition in a subject disclosed herein comprise administering to the subject the pharmaceutical composition.

In some aspects, the present disclosure provides a composition which can be administered by a parenteral, topical, intravenous, oral, subcutaneous, intra-arterial, intradermal, transdermal, rectal, intracranial, intraperitoneal, intranasal, intratumoral, intramuscular route, or as an inhalant. In some aspects, the pharmaceutical composition comprising EVs is administered intravenously, e.g. by injection. In some aspects, the composition can be administered by an intrathecal route. In some aspects, the composition can be administered by a cerebroventricular route. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the modified exosomes are formulated into ointments, salves, gels, or creams as generally known in the art.

In some aspects, the EVs are administered intravenously to the circulatory system of the subject. In some aspects, the EVs are infused in suitable liquid and administered into a vein of the subject. In some aspects, the EVs are administered intra-arterially to the circulatory system of the subject. In some aspects, the EVs are infused in suitable liquid and administered into an artery of the subject. In some aspects, the EVs are administered to the subject by intrathecal administration. In some aspects, the EVs are administered via an injection into the spinal canal, or into the subarachnoid space so that it reaches the cerebrospinal fluid (CSF). In some aspects, the EVs are administered intratumorally into one or more tumors of the subject. In some aspects, the EVs are administered to the subject by intranasal administration. In some aspects, the EVs can be insufflated through the nose in a form of either topical administration or systemic administration. In certain aspects, the EVs are administered as nasal spray.

In some aspects, the EVs are administered to the subject by intraperitoneal administration. In some aspects, the EVs are infused in suitable liquid and injected into the peritoneum of the subject. In some aspects, the intraperitoneal administration results in distribution of the EVs to the lymphatics. In some aspects, the intraperitoneal administration results in distribution of the EVs to the thymus, spleen, and/or bone marrow. In some aspects, the intraperitoneal administration results in distribution of the EVs to one or more lymph nodes. In some aspects, the intraperitoneal administration results in distribution of the EVs to one or more of the cervical lymph node, the inguinal lymph node, the mediastinal lymph node, or the sternal lymph node. In some aspects, the intraperitoneal administration results in distribution of the EVs to the pancreas.

In some aspects, the EVs, e.g., exosomes, are administered to the subject by periocular administration. In some aspects, the s are injected into the periocular tissues. Periocular drug administration includes the routes of subconjunctival, anterior sub-Tenon's, posterior sub-Tenon's, and retrobulbar administration.

In some aspects, the treatment is prophylactic. In some aspects, the EVs for the present disclosure are used to induce an immune response. In some aspects, the EVs for the present disclosure are used to vaccinate a subject.

In some aspects, the disease or condition is a cancer, a fibrosis, a hemophilia, diabetes, a growth factor deficiency, an eye disease, a Pompe disease, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, al antitrypsin deficiency, and a hypercholesterolemia.

In some aspects, the disease or disorder is a graft-versus-host disease (GvHD). In some aspects, the disease or disorder that can be treated with the present disclosure is an autoimmune disease. Non-limiting examples of autoimmune diseases include: multiple sclerosis, peripheral neuritis, Sjogren's syndrome, rheumatoid arthritis, alopecia, autoimmune pancreatitis, Behcet's disease, Bullous pemphigoid, Celiac disease, Devic's disease (neuromyelitis optica), Glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, and combinations thereof.

In some aspects, the disease or disorder is an infectious disease. In certain aspects, the disease or disorder is an oncogenic virus. In some aspects, infectious diseases that can be treated with the present disclosure includes, but not limited to, Human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikungunya virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof.

In some aspects, the cancer is bladder cancer, cervical cancer, renal cell cancer, testicular cancer, colorectal cancer, lung cancer, head and neck cancer, ovarian, lymphoma, liver cancer, glioblastoma, melanoma, myeloma, leukemia, pancreatic cancer, or combinations thereof.

In certain aspects, the cancer is associated with increased expression of a STAT6 protein. Non-limiting examples of cancers that can be treated with the present disclosure include a colorectal cancer, lung cancer (e.g., non-small cell lung cancer (NSCLC)), pancreatic cancer (e.g., pancreatic ductal adenocarcinoma (PDAC)), leukemia, uterine cancer, ovarian cancer, bladder cancer, bile duct cancer, gastric cancer, or any combination thereof. In some aspects, the cancer is selected from colon adenocarcinoma, rectum adenocarcinoma, pancreatic adenocarcinoma, pancreatic ductal adenocarcinoma (PDAC), ovarian serous cystadenocarcinoma, acute myelid leukemia, testicular cancer (e.g., testicular germ cell tumors, seminoma, non-seminoma and choriocarcinoma), lung adenocarcinoma, brain lower grade glioma, glioblastoma multiforme, uveal melanoma, thyroid carcinoma, uterine corpus endometrial carcinoma, uterine carcinosarcoma, pheochromocytoma, paraganglioma, and any combination thereof. In certain aspects, the cancer is a myeloid-rich cancer. In some aspects, the cancer comprises a liver cancer. In some aspects, the cancer comprises hepatocellular cancer (HCC). In some aspects, the cancer comprises pancreatic ductal adenocarcinoma (PDAC), in some aspects, the cancer comprises colorectal carcinoma (CRC). In some aspects, the cancer comprises ovarian cancer. In some aspects, the cancer comprises leptomeningeal cancer.

When administered to a subject with a cancer, in certain aspects, EVs of the present disclosure can up-regulate an immune response and enhance the tumor targeting of the subject's immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors”. In some aspects, the cancer being treated is characterized by low levels or undetectable levels of leukocyte infiltration into the tumor microenvironment, or so-called “cold tumors” or “non-inflammatory tumors”. In some aspects, an EV is administered in an amount and for a time sufficient to convert a “cold tumor” into a “hot tumor”, i.e., said administering results in the infiltration of leukocytes (such as T-cells) into the tumor microenvironment. In certain aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. In other term, “distal tumor” or “distant tumor” refers to a tumor that has spread from the original (or primary) tumor to distant organs or distant tissues, e.g., lymph nodes. In some aspects, the EVs of the disclosure treats a tumor after the metastatic spread.

All of the references cited above, as well as all references cited herein, are incorporated herein by reference in their entireties.

The following examples are offered by way of illustration and not by way of limitation.

### Examples

The disclosure is further illustrated by the following examples. The examples are provided for illustrative purposes only, and are not to be construed as limiting the scope or content of the disclosure in any way. The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); Green & Sambrook et al., Molecular Cloning: A Laboratory Manual, 4th Edition (Cold Spring Harbor Laboratory Press, 2012); Colowick & Kaplan, Methods In Enzymology (Academic Press); Remington: The Science and Practice of Pharmacy, 22nd Edition (Pharmaceutical Press, 2012); Sundberg & Carey, Advanced Organic Chemistry: Parts A and B, 5th Edition (Springer, 2007).

### Example 1: Exemplary Composition 04 Formulation Development

A drug product formulation for exosomes loaded with antisense oligomers will be developed. The C-03 drug product formulation will be used as the starting point. Phosphate buffer concentration (5 mM potassium phosphate monobasic and 15 mM sodium phosphate dibasic) and pH (pH 7.2) will remain unchanged relative to the C-03 drug product. Sodium chloride concentrations will tested at concentrations ranging from about 50 mM to about 150 mM, and sucrose concentrations will be tested at concentrations from about 2.5% to 5% (about 73 mM to about 146 mM). Conditions will be monitored. Various ASO-loaded exosome constructs will be analyzed, including exosomes loaded with ASOs targeting STAT6, as disclosed herein. In aqueous Tris EDTA buffer or pure water the ASO dissociates from the exosomes, and thus a combination of sodium chloride and sucrose is likely to play a role in retaining the loaded ASO.

### Example 2: Filtration of Compositions Comprising Exosomes Loaded with ASOs

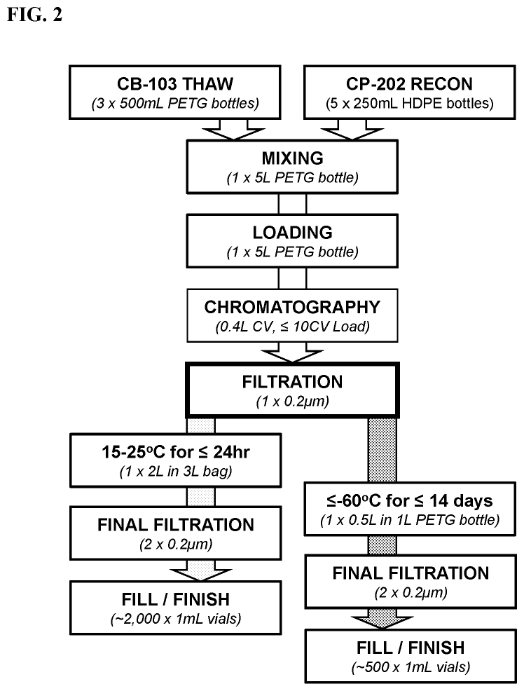

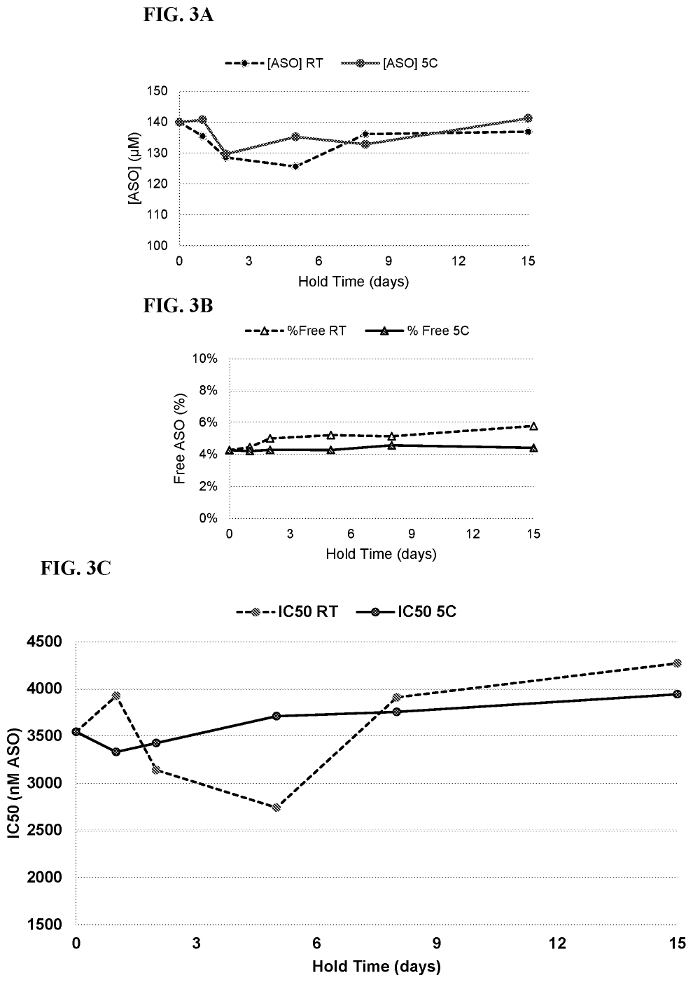

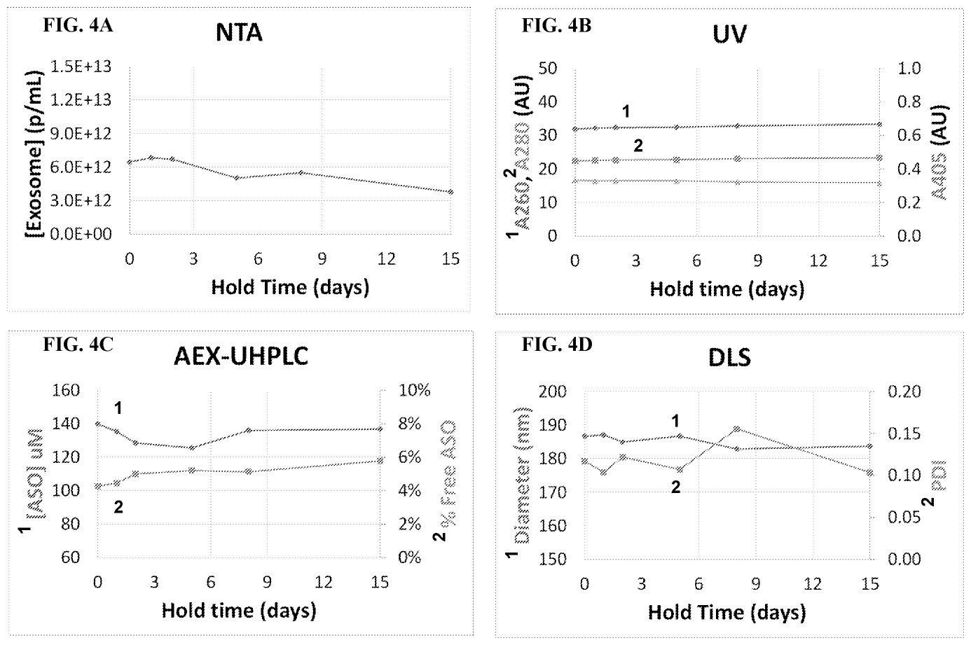

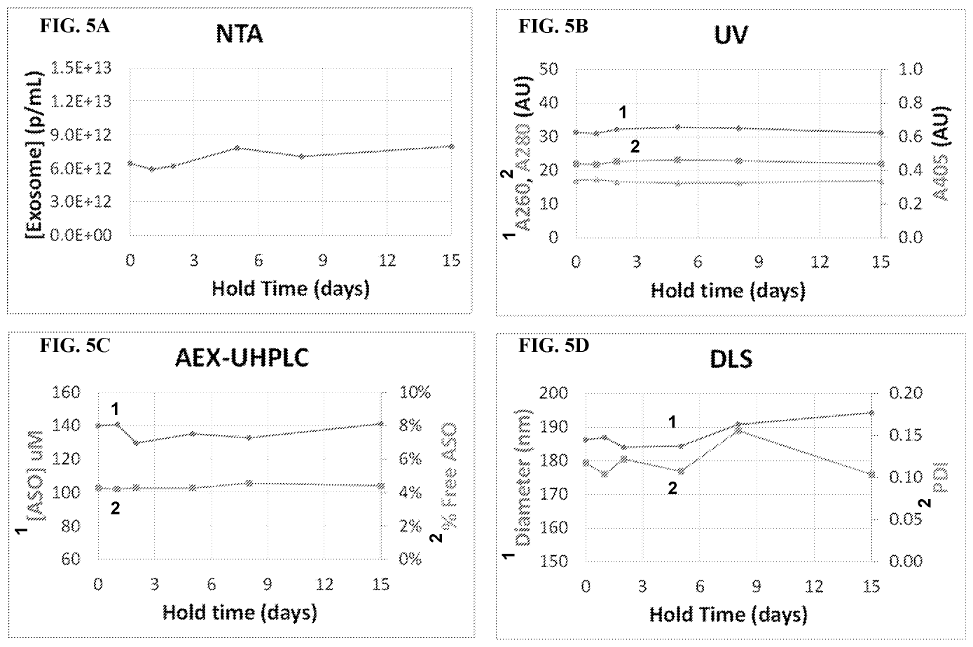

Exosomes loaded with ASOs targeting STAT6, as disclosed herein, were purified according to the protocol shown in FIG. 2. Following the filtrating step, samples were held at room temperature or 5° C. for 15 days, and ASO concentration (FIG. 3A), free ASO percent (FIG. 3B), and IC50 (FIG. 7C) were analyzed. Material was held at 20-22° C. (room temperature) and 5° C. for 15 days and tested throughout hold time for ASO content, both total and free, A260, A280, A405, in vitro potency with the STAT6 mRNA knock down cell reporter assay (FIGS. 4B and 5B), NTA for particle concentration (FIGS. 4A and 5A), and DLS (FIGS. 4D and 5D) to measure mean particle diameter and polydispersity index. Total ASO content measured by AEX-UPLC (FIG. 4C) indicates stability across a 15 day (about 2 weeks) hold at both hold temperatures. Percent free of ASO increases slightly as a function of hold time, while 5° C. hold seems to reduce percent free ASO. In vitro potency data shows IC50 values comparable across 8-day hold for RT. 5° C. trends towards less potent with longer hold, however changes are slight.

Following the room temperature hold (20-22° C.), NTA shows stability through 8 day hold. There were no changes observed in A260, A280, and A405, which indicates no loss of product through precipitation or adsorption to hold container, nor increase in turbidity. DLS shows so change in mean particle diameter indicating no observed aggregations across the hold.

Following the 5° C. hold, NTA shows stability through 15 day hold, with no changes observed in A260, A280, and A405, which indicates no loss of product through precipitation or adsorption to hold container, nor increase in turbidity. DLS shows no change in mean particle diameter to 8-day hold, indicating no observed aggregations across the hold. Day-15 hold shows a slight increase in diameter.

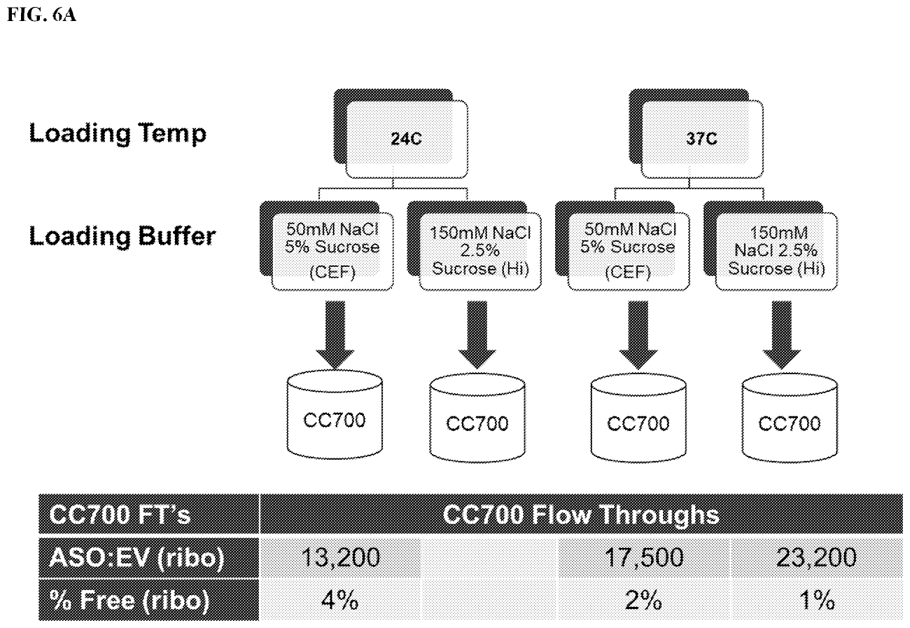

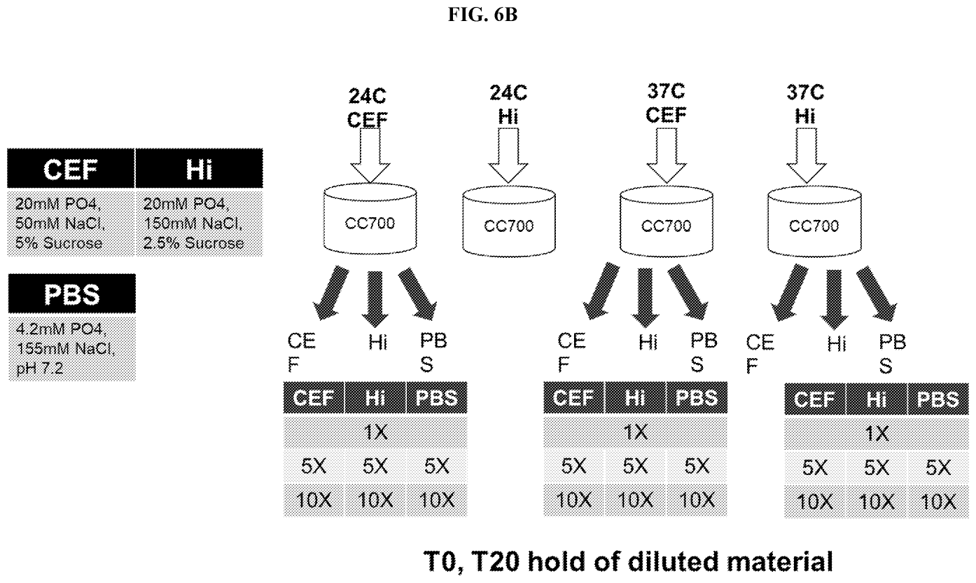

FIG. 6A provides a schematic diagram for a protocol that demonstrates differences in ASO/EV of loaded and cleaned up material when loading at room temperature as compared to loading at 37° C. and in higher salt (150 mM NaCl) as compared to in lower salt (50 mM NaCl) conditions. The highest salt condition when loading at 37° C. yields the highest ASO/EV in the cleaned up material. This ratio decreases when loading at 37° C., but at lower salt concentrations. An additional decrease in the ASO/EV is observed when reducing the loading temperature to 24° C. (or room temp, which ranges 20-25° C.). The percent free ASO shows that capto core is able to clean up the free ASO independent of the loading temperature and salt concentration, indicating that this method has robust cleanup capabilities.

FIG. 6B provides a schematic diagram for a protocol that highlights clean up capabilities of Capto Core for the three conditions run, above, and the effect of dilution factor, diluent (CEF, HI, and PBS), and hold time of diluted material on ASO's that dissociate from exosomes after they have been cleaned up from Capto Core. PBS has a similar salt concentration to a common and potential diluent (0.9% saline) that can be used in the clinic to dilute the exoASO prior to injection. The percent free ASO indicates that when diluting loaded and cleaned up material in a buffer containing a lower salt concentration, more ASO's dissociate from the EV's (Table 3). The dissociation becomes more drastic as the dilution factor increases. However, when diluting material in the higher salt containing buffers (150 mM NaCl), there is no dissociation of ASO's observed. When the diluted cleaned up material is held for 20 hours at room temperature, additional dissociation is observed only when the diluent contains lower salt concentrations (50 mM). For the higher salt containing diluents, no dissociation is observed when the diluted material is held for 20 hours at room temperature. These data indicate that the stability of the loaded exosomes can be optimized by maintaining a salt concentration.

TABLE 3
Capto Core Analysis

| | % Free in Sup t0 | | | | | | | | | | | |
|---|---|---|---|---|---|---|---|---|---|---|---|---|
| | Loading Buffer | | | | | | | | | | | |
| | CEF | | | | | | Hi | | | | | |
| | Loading Temperature | | | | | | | | | | | |
| | 22 C. | | | 37 C. | | | RT | | | 37 C. | | |
| Diluent | CEF | Hi | PBS | CEF | Hi | PBS | CEF | Hi | PBS | CEF | Hi | PBS |
| 1X | 4% | | | | | | 2% | | | 1% | | |
| 5X | 14% | 2% | 5% | | | | 11% | 3% | 2% | 6% | 1% | 1% |
| 10X | 14% | 2% | 2% | | | | 15% | 2% | 3% | 10% | 1% | 1% |

% Free in Sup t20

| CEF | | | | | | Hi | | | | | |
|---|---|---|---|---|---|---|---|---|---|---|---|
| 22 C. | | | 37 C. | | | RT | | | 37 C. | | |
| CEF | Hi | PBS | CEF | Hi | PBS | CEF | Hi | PBS | CEF | Hi | PBS |
| 7% | | | | | | 2% | | | 1% | | |
| 17% | 2% | 2% | | | | 14% | 2% | 2% | 7% | 1% | 1% |
| 29% | 1% | 1% | | | | 23% | 2% | 3% | 15% | 1% | 1% |

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific aspects have been illustrated and described, the above specification is not restrictive. It will be appreciated that various changes can be made without departing from the spirit and scope of the disclosure(s).

What is claimed is:

1. A pharmaceutical composition comprising an extracellular vesicle comprising an antisense oligonucleotide (ASO), wherein the ASO comprises a contiguous nucleotide sequence of 10 to 30 nucleotides in length that is complementary to a nucleic acid sequence within a STAT6 transcript;
wherein the composition has an osmolarity of lower than about 450 mOsm/kg.

2. The composition of claim 1, wherein the osmolarity of the composition is at least about 300 to about 450 mOsm/kg, at least about 310 to about 450 mOsm/kg, at least about 320 to about 450 mOsm/kg, at least about 330 to about 450 mOsm/kg, at least about 340 to about 450 mOsm/kg, at least about 350 to about 450 mOsm/kg, at least about 355 to about 450 mOsm/kg, at least about 360 to about 450 mOsm/kg, at least about 365 to about 450 mOsm/kg, at least about 365 to about 445 mOsm/kg, at least about 365 to about 440 mOsm/kg, at least about 365 to about 435 mOsm/kg, at least about 365 to about 430 mOsm/kg, at least about 365 to about 425 mOsm/kg, at least about 370 to about 420 mOsm/kg, at least about 375 to about 415 mOsm/kg, at least about 380 to about 410 mOsm/kg, at least about 385 to about 405 mOsm/kg, at least about 390 to about 400 mOsm/kg, at least about 395 to about 400 mOsm/kg, or at least about 390 to about 395 mOsm/kg.

3. The composition of claims 1 or 2, wherein the osmolarity of the composition is at least about 365 to about 425 mOsm/kg.

4. The composition of claim 1 or 2, wherein the osmolarity of the composition is about 365 mOsm/kg, about 370 mOsm/kg, about 375 mOsm/kg, about 380 mOsm/kg, about 385 mOsm/kg, about 390 mOsm/kg, about 395 mOsm/kg, about 400 mOsm/kg, about 405 mOsm/kg, about 410 mOsm/kg, about 415 mOsm/kg, about 420 mOsm/kg, or about 425 mOsm/kg.

5. The composition of any one of claims 1 to 4, wherein the osmolarity of the composition is about 395 mOsm/kg.

6. The composition of any one of claims 1 to 5, wherein the extracellular vesicle is an exosome.

7. The composition of any one of claims 1 to 6, wherein the composition is capable of being stored for at least about 4 hours, at least about 5 hours, at least about 6 hours, at least about 7 hours, at least about 8 hours, at least about 9 hours, at least about 10 hours, at least about 11 hours, at least about 12 hours, at least about 15 hours, at least about 20 hours, at least about 24 hours, at least about 2 days, at least about 3 days, at least about 4 days, at least about 5 days, at least about 6 days, or at least about 7 days at a temperature of 25° C.

8. The composition of any one of claims 1 to 7, wherein the composition is capable of being frozen and thawed, wherein the thawed composition has a pH of about 7.2.

9. The composition of any one of claims 1 to 8, wherein the composition has a pH of 7.0, 7.1, 7.2, 7.3, or 7.4.

10. The composition of any one of claims 1 to 9, wherein the composition has a pH of 7.2.

11. The composition of any one of claims 1 to 10, wherein the pI is in the range of about 1 to about 6.5.

12. The composition of any one of claims 1 to 11, wherein the composition has (i) reduced aggregates, (ii) improved stability of the EV, (iii) improved integrity of the EV architecture, (iv) improved stability of engineered proteins contained on or in EVs, (v) improved filterability, and (vi) reduces dissociation of the ASO.

13. The composition of any one of claims 1 to 11, which further comprises (i) a saccharide, (ii) sodium phosphate, (iii) potassium phosphate, (iv) sodium phosphate, or (v) any combination thereof.

14. The composition of claim 13, wherein the saccharide comprises a monosaccharide, a disaccharide, a trisaccharide, an oligosaccharide, a polysaccharide, a sugar alcohol, or any combination thereof.

15. The composition of claim 13 or 14, wherein the saccharide has a molecular weight of from about 180.00 g/mol to about 380.00 g/mol.

16. The composition of any one of claims 13 to 15, wherein the saccharide comprises lactose, glucose, sucrose, trehalose, and/or combinations thereof.

17. The composition of any one of claims 13 to 16, wherein the saccharide is a sugar alcohol having a molecular weight of from about 90.00 g/mol to about 190.00 g/mol.

18. The composition of claim 17, wherein the sugar alcohol comprises glycerol, sorbitol, mannitol, xylitol, and/or combinations thereof.

19. The composition of any one of claims 13 to 18, wherein the saccharide is a sucrose or a trehalose.

20. The composition of any one of claims 13 to 19, wherein the saccharide is present in the composition at a concentration of about 5% w/v.

21. The composition of any one of claims 13 to 20, wherein the composition has a conductivity between about 6 mS/cm+/−10% and about 10 mS/cm+/−10%.

22. The composition of claim 21, wherein the conductivity is between 6 mS/cm and about 7 mS/cm, between about 7 mS/cm and about 8 mS/cm, between about 8 mS/cm and about 9 mS/cm, or between about 9 mS/cm and about 10 mS/cm.

23. The composition of claim 21 or 22, wherein the conductivity is about 6 mS/cm, about 7 mS/cm, about 8 mS/cm, about 9 mS/cm, or about 10 mS/cm.

24. The composition of any one of claims 13 to 23, wherein the sodium chloride is present in the composition at a concentration of between about 50 mM and about 150 mM.

25. The composition of claim 24, wherein the concentration of sodium chloride is between about 50 mM to about 140 mM, between about 60 mM to about 130 mM, between about 70 mM to about 120 mM, between about 80 mM to about 110 mM, between about 90 mM to about 100 mM, between about 100 mM to about 110 mM, between about 95 mM to about 105 mM, between about 95 mM to about 110 mM, or between about 90 mM to about 105 mM.

26. The composition of claim 24 or 25, wherein the concentration of sodium chloride is about 50 mM, about 60 mM, about 70 mM, about 80 mM, about 90 mM, about 100 mM, about 110 mM, about 120 mM, about 130 mM, about 140 mM, or about 150 mM.

27. The composition of any one of claims 24 to 26, wherein the concentration of sodium chloride is about 95 mM, about 96 mM, about 97 mM, about 98 mM, about 99 mM, about 100 mM, about 101 mM, about 102 mM, about 103 mM, about 104 mM, or about 105 mM.

28. The composition of any one of claims 13 to 27, wherein the potassium phosphate is present in the composition at a concentration of between about 1 mM to about 10 mM, between about 2 mM to about 9 mM, between about 3 mM to about 8 mM, between about 4 mM to about 7 mM, between about 5 mM to about 6 mM, or between about 4 mM to about 5 mM.

29. The composition of claim 28, wherein the concentration of the potassium phosphate is about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, or about 5.5 mM.

30. The composition of claim 28 or 29, wherein the concentration of the potassium phosphate is about 5 mM.

31. The composition of any one of claims 13 to 30, wherein the potassium phosphate is potassium phosphate monobasic.

32. The composition of any one of claims 13 to 31, wherein the sodium phosphate is present in the composition at a concentration of between about 5 mM to about 30, between about 10 mM to about 20 mM, between about 11 mM to about 19 mM, between about 12 mM to about 18 mM, between about 13 mM to about 17 mM, between about 14 mM to about 16 mM, between about 15 mM to about 16 mM, or between about 14 mM to about 15 mM.

33. The composition of claim 32, wherein the sodium phosphate is present in the composition at a concentration of about 14.5 mM, about 14.6 mM, about 14.7 mM, about 14.8 mM, about 14.9 mM, about 15.0 mM, about 15.1 mM about 15.2 mM, about 15.3 mM, about 15.4, mM, or about 15.5 mM.

34. The composition of claim 32 or 33, wherein the concentration of the sodium phosphate is about 15 mM.

35. The composition of any one of claims 13 to 34, wherein the sodium phosphate is sodium phosphate dibasic heptahydrate.

36. The composition of any one of claims 1 to 35, wherein the composition is not lyophilized.

37. The composition of any one of claims 1 to 36, wherein the composition does not comprise a chelating agent.

38. The composition of any one of claims 1 to 37, wherein the composition does not comprise albumin.

39. The composition of any one of claims 1 to 38, comprising
a. a sucrose at a concentration of about 5% w/v,
b. sodium chloride at a concentration of about 100 mM;
c. a potassium phosphate monobasic at a concentration of about 5 mM;
d. a sodium phosphate dibasic heptahydrate at a concentration of about 15 mM;
wherein the composition is in a solution at a pH of 7.2; and
wherein the composition comprises an osmolarity of about 365 to about 425 mOsm/kg.

40. The composition of any one of claims 1 to 38, comprising,
a. a sucrose at a concentration of about 5% w/v,
b. sodium chloride at a concentration of about 100 mM;
c. a potassium phosphate monobasic at a concentration of about 5 mM;
d. a sodium phosphate dibasic heptahydrate at a concentration of about 15 mM;
wherein the composition is in a solution at a pH of 7.2; and
wherein the composition comprises an osmolarity of about 395 mOsm/kg.

41. The composition of any one of claims 1 to 38, comprising
a. a sucrose at a concentration of about 146 mM,
b. sodium chloride at a concentration of about 100 mM;
c. a potassium phosphate monobasic at a concentration of about 5 mM;
d. a sodium phosphate dibasic heptahydrate at a concentration of about 15 mM;
wherein the composition is in a solution at a pH of 7.2; and
wherein the composition comprises an osmolarity of about 365 to about 425 mOsm/kg.

42. The composition of any one of claims 1 to 38, comprising,
a. a sucrose at a concentration of about 146 mM,
b. sodium chloride at a concentration of about 100 mM;
c. a potassium phosphate monobasic at a concentration of about 5 mM;
d. a sodium phosphate dibasic heptahydrate at a concentration of about 15 mM;
wherein the composition is in a solution at a pH of 7.2; and
wherein the composition comprises an osmolarity of about 395 mOsm/kg.

43. The composition of any one of claims 1 to 42, wherein the composition is capable of being stored at a temperature of from about −20° C. to about −80° C., wherein the stability of the extracellular vesicle is not reduced.

44. The composition of claim 43, wherein the composition can be stored for about one week, about two weeks, about three weeks, about four weeks, about one month, about two months, about three months, about four months, about five months, about six months, about seven months, about eight months, about nine months, about ten months, about 11 months, about 12 months, about one year, about two years, about three years, or about four years.

45. The composition of any one of claims 1 to 44, wherein the extracellular vesicle further comprises a scaffold protein.

46. The composition of claim 45, wherein the scaffold protein is Scaffold X.

47. The composition of claim 45 or 46, wherein a payload is linked to the scaffold protein.

48. The composition of claim 47, wherein the payload is linked to the scaffold protein by a linker.

49. The composition of claim 48, wherein the linker is a polypeptide.

50. The composition of claim 49, wherein the linker is a non-polypeptide moiety.

51. The composition of any one of claims 46 to 50, wherein Scaffold X is a scaffold protein that is capable of anchoring the payload on the exterior surface of the extracellular vesicle.

52. The composition of any one of claims 46 to 51, wherein the scaffold protein comprises prostaglandin F2 receptor negative regulator (the PTGFRN protein).

53. The composition of any one of claims 45 to 52, wherein the scaffold protein comprises the PTGFRN protein or a fragment thereof.

54. The composition of claim 45 to 52, wherein the scaffold protein comprises an amino acid sequence as set forth in any one of SEQ ID NOs: 1 and 6-12.

55. The composition of any one of claims 45 to 54, wherein the scaffold protein comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 1.

56. The composition of any one of claims 1 to 55, wherein the composition can be administered by a parenteral, topical, intravenous, oral, subcutaneous, intra-arterial, intradermal, transdermal, rectal, intracranial, intraperitoneal, intranasal, intratumoral, intramuscular intrathecal, or cerebroventricular route, or as an inhalant.

57. The composition of any one of claims 1 to 56, wherein the ASO comprises a nucleic acid sequence selected from SEQ ID NOs: 91-193

58. The composition of any one of claims 1 to 56, comprising:
(a) An extracellular vesicles comprising an ASO, wherein the ASO comprises a nucleic acid sequence selected from SEQ ID NOs: 91-193;
(b) Sucrose at a concentration of about 50 mM to about 150 mM;
(c) Sodium chloride at a concentration of about 100 mM to about 200 mM;
(d) Potassium phosphate monobasic at a concentration of about 5 mM; and
(e) Sodium phosphate dibasic at a concentration of about 15 mM,
wherein the pH of the composition is about 7.2; and
wherein the composition comprises an osmolarity of at least about 365 to about 425 mOsm/kg.

59. The composition of any one of claims 1 to 56, comprising:
(a) An extracellular vesicles comprising an ASO, wherein the ASO comprises a nucleic acid sequence selected from SEQ ID NOs: 91-193;
(b) Sucrose at a concentration of about 4% to about 6%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
wherein the pH of the composition is about 7.2; and
wherein the composition comprises an osmolarity of at least about 365 to about 425 mOsm/kg.

60. The composition of any one of claims 1 to 56, comprising:
(a) Extracellular vesicles comprising an ASO, wherein the ASO comprises a nucleic acid sequence selected from SEQ ID NOs: 91-193;
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
wherein the pH of the composition is about 7.2; and
wherein the composition comprises an osmolarity of at least about 365 to about 425 mOsm/kg.

61. The composition of any one of claims 1 to 56, comprising:
(a) Extracellular vesicles comprising an ASO, wherein the ASO comprises a nucleic acid sequence selected from SEQ ID NOs: 91-193;
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
wherein the pH of the composition is about 7.2; and
wherein the composition comprises an osmolarity of at least about 365 to about 425 mOsm/kg.

62. The composition of any one of claims 1 to 56, comprising:
(a) Extracellular vesicles comprising an ASO, wherein the ASO comprises a nucleic acid sequence selected from SEQ ID NOs: 91-193;
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
wherein the pH of the composition is about 7.2; and
wherein the composition comprises an osmolarity of about 395 mOsm/kg.

63. The composition of any one of claims 1 to 56, comprising:
(a) Extracellular vesicles comprising an ASO, wherein the ASO comprises a nucleic acid sequence selected from SEQ ID NOs: 91-193;
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
wherein the pH of the composition is about 7.2; and
wherein the composition comprises an osmolarity of about 395 mOsm/kg.

64. The method of any one of claims 58 to 63, wherein the extracellular vesicle is an exosome.

65. The composition of any one of claims 1 to 64, wherein the ASO comprises the nucleic acid sequence set forth in SEQ ID NO: 144.

66. The composition of any one of claims 1 to 64, wherein the ASO comprises the nucleic acid sequence set forth in SEQ ID NO: 145.

67. The composition of any one of claims 1 to 64, wherein the ASO comprises the nucleic acid sequence set forth in SEQ ID NO: 193.

68. The composition of any one of claims 1 to 64, wherein the ASO comprises the nucleic acid sequence set forth in SEQ ID NO: 185.

69. The composition of any one of claims 1 to 68, wherein the ASO is associated with the extracellular vesicles by a linker.

70. The composition of claim 69, wherein the linker comprises cholesterol, tocopherol, a fatty acid, or any combination thereof.

71. The composition of claim 69 or 70, wherein the linker is a cleavable linker.

72. The composition of any one of claims 48 to 71, wherein the linker is a cleavable linker.

73. A method of treating a disease or a condition in a subject in need thereof comprising administering to the subject the composition of any one of claims 1 to 72.

74. The method of claim 73, wherein the disease or condition is a cancer, a fibrosis, a hemophilia, diabetes, a growth factor deficiency, an eye disease, a Pompe disease, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, al antitrypsin deficiency, and a hypercholesterolemia.

75. The method of claim 74, wherein the cancer is bladder cancer, cervical cancer, renal cell cancer, testicular cancer, colorectal cancer, lung cancer, head and neck cancer, ovarian, lymphoma, liver cancer, glioblastoma, melanoma, myeloma, leukemia, pancreatic cancer, or combinations thereof.

76. The pharmaceutical composition of any one of claims 1 to 72 for treating a disease or a condition in a subject in need thereof.

77. Use of the composition of any one of claims 1 to 72 in the manufacture of a medicament for treating a disease or a condition.

78. A method of preparing the pharmaceutical composition of any one of claims 1 to 72 comprising combining (i) an extracellular vesicle comprising an ASO and (ii) a salt or a saccharide; wherein the ASO comprises a contiguous nucleotide sequence of 10 to 30 nucleotides in length that is complementary to a nucleic acid sequence within a STAT6 transcript; and wherein the composition comprises an osmolarity lower than about 450 mOsm/kg.

79. The method of claim 78, wherein the salt comprises
a. sodium chloride;
b. a potassium phosphate;
c. a sodium phosphate, or
d. any combination thereof.


SEQUENCE LISTING (193 sequences)

SEQ ID NO: 1 - protein, 879 aa, Artificial Sequence
  Full length PTGFRN Protein
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 847 aa, Artificial Sequence
  STAT6 Protein Sequence
MSLWGLVSKMPPEKVQRLYVDFPQHLRHLLGDWLESQPWEFLVGSDAFCCNLASALLSDTVQHLQASVGEQGEGSTILQHISTLESIYQRDPLKLVATFRQILQGEKKAVMEQFRHLPMPFHWKQEELKFKTGLRRLQHRVGEIHLLREALQKGAEAGQVSLHSLIETPANGTGPSEALAMLLQETTGELEAAKALVLKRIQIWKRQQQLAGNGAPFEESLAPLQERCESLVDIYSQLQQEVGAAGGELEPKTRASLTGRLDEVLRTLVTSCFLVEKQPPQVLKTQTKFQAGVRFLLGLRFLGAPAKPPLVRADMVTEKQARELSVPQGPGAGAESTGEIINNTVPLENSIPGNCCSALFKNLLLKKIKRCERKGTESVTEEKCAVLFSASFTLGPGKLPIQLQALSLPLVVIVHGNQDNNAKATILWDNAFSEMDRVPFVVAERVPWEKMCETLNLKFMAEVGTNRGLLPEHFLFLAQKIFNDNSLSMEAFQHRSVSWSQFNKEILLGRGFTFWQWFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATIKMTVERDQPLPTPELQMPTMVPSYDLGMAPDSSMSMQLGPDMVPQVYPPHSHSIPPYQGLSPEESVNVLSAFQEPHLQMPPSLGQMSLPFDQPHPQGLLPCQPQEHAVSSPDPLLCSDVTMVEDSCLSQPVTAFPQGTWIGEDIFPPLLPPTEQDLTKLLLEGQGESGGGSLGAQPLLQPSHYGQSGISMSHMDLRANPSW

SEQ ID NO: 3 - DNA, 3963 bp, Artificial Sequence
  STAT6 mRNA Sequence
ggggcagccactgcttacactgaagagggaggacgggagaggagtgtgtgtgtgtgtgtgtgtgtgtgtgtgtatgtatgtgtgtgctttatcttatttttctttttggtggtggtggtggaaggggggaggtgctagcagggccagccttgaactcgctggacagagctacagacctatggggcctggaagtgcccgctgagaaagggagaagacagcagaggggttgccgaggcaacctccaagtcccagatcatgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtaggggagatccaccttctccgagaagccctgcagaagggggctgaggctggccaagtgtctctgcacagcttgatagaaactcctgctaatgggactgggccaagtgaggccctggccatgctactgcaggagaccactggagagctagaggcagccaaagccctagtgctgaagaggatccagatttggaaacggcagcagcagctggcagggaatggcgcaccgtttgaggagagcctggccccactccaggagaggtgtgaaagcctggtggacatttattcccagctacagcaggaggtaggggcggctggtggggagcttgagcccaagacccgggcatcgctgactggccggctggatgaagtcctgagaaccctcgtcaccagttgcttcctggtggagaagcagcccccccaggtactgaagactcagaccaagttccaggctggagttcgattcctgttgggcttgaggttcctgggggccccagccaagcctccgctggtcagggccgacatggtgacagagaagcaggcgcgggagctgagtgtgcctcagggtcctggggctggagcagaaagcactggagaaatcatcaacaacactgtgcccttggagaacagcattcctgggaactgctgctctgccctgttcaagaacctgcttctcaagaagatcaagcggtgtgagcggaagggcactgagtctgtcacagaggagaagtgcgctgtgctcttctctgccagcttcacacttggccccggcaaactccccatccagctccaggccctgtctctgcccctggtggtcatcgtccatggcaaccaagacaacaatgccaaagccactatcctgtgggacaatgccttctctgagatggaccgcgtgccctttgtggtggctgagcgggtgccctgggagaagatgtgtgaaactctgaacctgaagttcatggctgaggtggggaccaaccgggggctgctcccagagcacttcctcttcctggcccagaagatcttcaatgacaacagcctcagtatggaggccttccagcaccgttctgtgtcctggtcgcagttcaacaaggagatcctgctgggccgtggcttcaccttttggcagtggtttgatggtgtcctggacctcaccaaacgctgtctccggagctactggtctgaccggctgatcattggcttcatcagcaaacagtacgttactagccttcttctcaatgagcccgacggaacctttctcctccgcttcagcgactcagagattgggggcatcaccattgcccatgtcatccggggccaggatggctctccacagatagagaacatccagccattctctgccaaagacctgtccattcgctcactgggggaccgaatccgggatcttgctcagctcaaaaatctctatcccaagaagcccaaggatgaggctttccggagccactacaagcctgaacagatgggtaaggatggcaggggttatgtcccagctaccatcaagatgaccgtggaaagggaccaaccacttcctaccccagagctccagatgcctaccatggtgccttcttatgaccttggaatggcccctgattcctccatgagcatgcagcttggcccagatatggtgccccaggtgtacccaccacactctcactccatccccccgtatcaaggcctctccccagaagaatcagtcaacgtgttgtcagccttccaggagcctcacctgcagatgccccccagcctgggccagatgagcctgccctttgaccagcctcacccccagggcctgctgccgtgccagcctcaggagcatgctgtgtccagccctgaccccctgctctgctcagatgtgaccatggtggaagacagctgcctgagccagccagtgacagcgtttcctcagggcacttggattggtgaagacatattccctcctctgctgcctcccactgaacaggacctcactaagcttctcctggaggggcaaggggagtcggggggagggtccttgggggcacagcccctcctgcagccctcccactatgggcaatctgggatctcaatgtcccacatggacctaagggccaaccccagttggtgatcccagctggagggagaacccaaagagacagctcttctactacccccacagacctgctctggacacttgctcatgccctgccaagcagcagatggggagggtgccctcctatccccacctactcctgggtcaggaggaaaagactaacaggagaatgcacagtgggtggagccaatccactccttcctttctatcattcccctgcccacctccttccagcactgactggaagggaagttcaggctctgagacacaccccaacatgcctgcacctgcagcgcgcacacgcacgcacacacacatacagagctctctgagggtgatggggctgagcaggaggggggctgggtaagagcacaggttagggcatggaaggcttctccgcccattctgacccagggcctaggacggataggcaggaacatacagacacatttacactagaggccagggatagaggatattgggtctcagccctaggggaatgggaagcagctcaagggaccctgggtgggagcataggaggggtctggacatgtggttactagtacaggttttgccctgattaaaaaatctcccaaagccccaaattcctgttagccaggtggaggcttctgatacgtgtatgagactatgcaaaagtacaagggctgagattcttcgtgtatagctgtgtgaacgtgtatgtacctaggatatgttaaatgtatagctggcaccttagttgcatgaccacatagaacatgtgtctatctgcttttgcctacgtgacaacacaaatttgggagggtgagacactgcacagaagacagcagcaagtgtgctggcctctctgacatatgctaacccccaaatactctgaatttggagtctgactgtgcccaagtgggtccaagtggctgtgacatctacgtatggctccacacctccaatgctgcctgggagccagggtgagagtctgggtccaggcctggccatgtggccctccagtgtatgagagggccctgcctgctgcatcttttctgttgccccatccaccgccagcttcccttcactcccctatcccattctccctctcaaggcaggggtcatagatcctaagccataaaataaattttattccaaaataacaaaataaataatctactgtacacaatctgaaaa

SEQ ID NO: 4-5 - skipped (2 entries)

SEQ ID NO: 6 - protein, 731 aa, Artificial Sequence
  PTGFRN Protein Fragment #1
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 7 - protein, 611 aa, Artificial Sequence
  PTGFRN Protein Fragment #2
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 8 - protein, 485 aa, Artificial Sequence
  PTGFRN Protein Fragment #3
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 9 - protein, 193 aa, Artificial Sequence
  PTGFRN Protein Fragment #7
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 10 - protein, 343 aa, Artificial Sequence
  PTGFRN Protein Fragment #4
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 11 - protein, 217 aa, Artificial Sequence
  PTGFRN Protein Fragment #5
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 12 - protein, 66 aa, Artificial Sequence
  PTGFRN Protein Fragment #6
SKPVFITVKMDVLNAFKYPLLIGVGLSTVIGLLSCLIGYCSSHWCCKKEVQETRRERRRLMSMEMD

SEQ ID NO: 13 - protein, 21 aa, Artificial Sequence
  PTGFRN Protein - Signal Peptide
MGRLASRPLLLALLSLALCRG

SEQ ID NO: 14-90 - skipped (77 entries)

SEQ ID NO: 91 - DNA, 15 bp, Artificial Sequence
  Synthetic sequence
ctggtgacgagggtt

SEQ ID NO: 92 - DNA, 15 bp, Artificial Sequence
  Synthetic sequence
cgctcacaccgcttg

SEQ ID NO: 93 - DNA, 15 bp, Artificial Sequence
  Synthetic sequence
aggctagtaacgtac

SEQ ID NO: 94 - DNA, 15 bp, Artificial Sequence
  Synthetic sequence
gaaggctagtaacgt

SEQ ID NO: 95 - DNA, 15 bp, Artificial Sequence
  Synthetic sequence
ggttccgtcgggctc

SEQ ID NO: 96 - DNA, 15 bp, Artificial Sequence
  Synthetic sequence
aggttccgtcgggct

SEQ ID NO: 97 - DNA, 15 bp, Artificial Sequence
  Synthetic sequence
aaggttccgtcgggc

SEQ ID NO: 98 - DNA, 15 bp, Artificial Sequence
  Synthetic sequence
aaaggttccgtcggg

SEQ ID NO: 99 - DNA, 15 bp, Artificial Sequence
  Synthetic sequence
gaaaggttccgtcgg

SEQ ID NO: 100 - DNA, 15 bp, Artificial Sequence
  Synthetic sequence
agaaaggttccgtcg

SEQ ID NO: 101 - DNA, 15 bp, Artificial Sequence
  Synthetic sequence
agtcgctgaagcgga

SEQ ID NO: 102 - DNA, 15 bp, Artificial Sequence
  Synthetic sequence
gagtcgctgaagcgg

SEQ ID NO: 103 - DNA, 15 bp, Artificial Sequence
  Synthetic sequence
cggattcggtccccc

SEQ ID NO: 104 - DNA, 15 bp, Artificial Sequence
  Synthetic sequence
ccggattcggtcccc

SEQ ID NO: 105 - DNA, 15 bp, Artificial Sequence
  Synthetic sequence
cccggattcggtccc

SEQ ID NO: 106 - DNA, 15 bp, Artificial Sequence
  Synthetic sequence
tcccggattcggtcc

SEQ ID NO: 107 - DNA, 15 bp, Artificial Sequence
  Synthetic sequence
gatcccggattcggt

SEQ ID NO: 108 - DNA, 15 bp, Artificial Sequence
  Synthetic sequence
agatcccggattcgg

SEQ ID NO: 109 - DNA, 15 bp, Artificial Sequence
  Synthetic sequence
aagatcccggattcg

SEQ ID NO: 110 - DNA, 15 bp, Artificial Sequence
  Synthetic sequence
tgatacggggggatg

SEQ ID NO: 111 - DNA, 15 bp, Artificial Sequence
  Synthetic sequence
cgtgtgcgcgctgca

SEQ ID NO: 112 - DNA, 16 bp, Artificial Sequence
  Synthetic sequence
actggtgacgagggtt

SEQ ID NO: 113 - DNA, 16 bp, Artificial Sequence
  Synthetic sequence
aactggtgacgagggt

SEQ ID NO: 114 - DNA, 16 bp, Artificial Sequence
  Synthetic sequence
ctcacaccgcttgatc

SEQ ID NO: 115 - DNA, 16 bp, Artificial Sequence
  Synthetic sequence
cggtcagaccagtagc

SEQ ID NO: 116 - DNA, 16 bp, Artificial Sequence
  Synthetic sequence
ctagtaacgtactgtt

SEQ ID NO: 117 - DNA, 16 bp, Artificial Sequence
  Synthetic sequence
gctagtaacgtactgt

SEQ ID NO: 118 - DNA, 16 bp, Artificial Sequence
  Synthetic sequence
ggctagtaacgtactg

SEQ ID NO: 119 - DNA, 16 bp, Artificial Sequence
  Synthetic sequence
aggctagtaacgtact

SEQ ID NO: 120 - DNA, 16 bp, Artificial Sequence
  Synthetic sequence
aaggctagtaacgtac

SEQ ID NO: 121 - DNA, 16 bp, Artificial Sequence
  Synthetic sequence
aagaaggctagtaacg

SEQ ID NO: 122 - DNA, 16 bp, Artificial Sequence
  Synthetic sequence
aaaggttccgtcgggc

SEQ ID NO: 123 - DNA, 16 bp, Artificial Sequence
  Synthetic sequence
agaaaggttccgtcgg

SEQ ID NO: 124 - DNA, 16 bp, Artificial Sequence
  Synthetic sequence
ttcggtcccccagtga

SEQ ID NO: 125 - DNA, 16 bp, Artificial Sequence
  Synthetic sequence
cggattcggtccccca

SEQ ID NO: 126 - DNA, 16 bp, Artificial Sequence
  Synthetic sequence
agatcccggattcggt

SEQ ID NO: 127 - DNA, 16 bp, Artificial Sequence
  Synthetic sequence
agcaagatcccggatt

SEQ ID NO: 128 - DNA, 16 bp, Artificial Sequence
  Synthetic sequence
tgatacggggggatgg

SEQ ID NO: 129 - DNA, 16 bp, Artificial Sequence
  Synthetic sequence
ttgatacggggggatg

SEQ ID NO: 130 - DNA, 16 bp, Artificial Sequence
  Synthetic sequence
agaggccttgatacgg

SEQ ID NO: 131 - DNA, 16 bp, Artificial Sequence
  Synthetic sequence
gggttagcatatgtca

SEQ ID NO: 132 - DNA, 17 bp, Artificial Sequence
  Synthetic sequence
tggatctcccctactcg

SEQ ID NO: 133 - DNA, 17 bp, Artificial Sequence
  Synthetic sequence
actggtgacgagggttc

SEQ ID NO: 134 - DNA, 17 bp, Artificial Sequence
  Synthetic sequence
gctcacaccgcttgatc

SEQ ID NO: 135 - DNA, 17 bp, Artificial Sequence
  Synthetic sequence
cgctcacaccgcttgat

SEQ ID NO: 136 - DNA, 17 bp, Artificial Sequence
  Synthetic sequence
tccgctcacaccgcttg

SEQ ID NO: 137 - DNA, 17 bp, Artificial Sequence
  Synthetic sequence
ttccgctcacaccgctt

SEQ ID NO: 138 - DNA, 17 bp, Artificial Sequence
  Synthetic sequence
accagtagctccggaga

SEQ ID NO: 139 - DNA, 17 bp, Artificial Sequence
  Synthetic sequence
gccggtcagaccagtag

SEQ ID NO: 140 - DNA, 17 bp, Artificial Sequence
  Synthetic sequence
agccggtcagaccagta

SEQ ID NO: 141 - DNA, 17 bp, Artificial Sequence
  Synthetic sequence
agaaggctagtaacgta

SEQ ID NO: 142 - DNA, 17 bp, Artificial Sequence
  Synthetic sequence
aaggttccgtcgggctc

SEQ ID NO: 143 - DNA, 17 bp, Artificial Sequence
  Synthetic sequence
aaaggttccgtcgggct

SEQ ID NO: 144 - DNA, 17 bp, Artificial Sequence
  Synthetic sequence
gaaaggttccgtcgggc

SEQ ID NO: 145 - DNA, 17 bp, Artificial Sequence
  Synthetic sequence
ctgagtcgctgaagcgg

SEQ ID NO: 146 - DNA, 17 bp, Artificial Sequence
  Synthetic sequence
tctgagtcgctgaagcg

SEQ ID NO: 147 - DNA, 17 bp, Artificial Sequence
  Synthetic sequence
atcccggattcggtccc

SEQ ID NO: 148 - DNA, 17 bp, Artificial Sequence
  Synthetic sequence
gatcccggattcggtcc

SEQ ID NO: 149 - DNA, 17 bp, Artificial Sequence
  Synthetic sequence
agatcccggattcggtc

SEQ ID NO: 150 - DNA, 17 bp, Artificial Sequence
  Synthetic sequence
aagatcccggattcggt

SEQ ID NO: 151 - DNA, 17 bp, Artificial Sequence
  Synthetic sequence
gcaagatcccggattcg

SEQ ID NO: 152 - DNA, 17 bp, Artificial Sequence
  Synthetic sequence
cggtcatcttgatggta

SEQ ID NO: 153 - DNA, 17 bp, Artificial Sequence
  Synthetic sequence
tgatacggggggatgga

SEQ ID NO: 154 - DNA, 17 bp, Artificial Sequence
  Synthetic sequence
ttgatacggggggatgg

SEQ ID NO: 155 - DNA, 17 bp, Artificial Sequence
  Synthetic sequence
cttgatacggggggatg

SEQ ID NO: 156 - DNA, 17 bp, Artificial Sequence
  Synthetic sequence
gaggccttgatacgggg

SEQ ID NO: 157 - DNA, 20 bp, Artificial Sequence
  Synthetic sequence
gggggtccctctgatatatg

SEQ ID NO: 158 - DNA, 20 bp, Artificial Sequence
  Synthetic sequence
gtggatctcccctactcggt

SEQ ID NO: 159 - DNA, 20 bp, Artificial Sequence
  Synthetic sequence
agcccaacaggaatcgaact

SEQ ID NO: 160 - DNA, 20 bp, Artificial Sequence
  Synthetic sequence
cgctcacaccgcttgatctt

SEQ ID NO: 161 - DNA, 20 bp, Artificial Sequence
  Synthetic sequence
ccgctcacaccgcttgatct

SEQ ID NO: 162 - DNA, 20 bp, Artificial Sequence
  Synthetic sequence
tccgctcacaccgcttgatc

SEQ ID NO: 163 - DNA, 20 bp, Artificial Sequence
  Synthetic sequence
agtttgccggggccaagtgt

SEQ ID NO: 164 - DNA, 20 bp, Artificial Sequence
  Synthetic sequence
aagggcacgcggtccatctc

SEQ ID NO: 165 - DNA, 20 bp, Artificial Sequence
  Synthetic sequence
aaagggcacgcggtccatct

SEQ ID NO: 166 - DNA, 20 bp, Artificial Sequence
  Synthetic sequence
caaagggcacgcggtccatc

SEQ ID NO: 167 - DNA, 20 bp, Artificial Sequence
  Synthetic sequence
acaaagggcacgcggtccat

SEQ ID NO: 168 - DNA, 20 bp, Artificial Sequence
  Synthetic sequence
ccggagacagcgtttggtga

SEQ ID NO: 169 - DNA, 20 bp, Artificial Sequence
  Synthetic sequence
tccggagacagcgtttggtg

SEQ ID NO: 170 - DNA, 20 bp, Artificial Sequence
  Synthetic sequence
agtagctccggagacagcgt

SEQ ID NO: 171 - DNA, 20 bp, Artificial Sequence
  Synthetic sequence
cggtcagaccagtagctccg

SEQ ID NO: 172 - DNA, 20 bp, Artificial Sequence
  Synthetic sequence
agccggtcagaccagtagct

SEQ ID NO: 173 - DNA, 20 bp, Artificial Sequence
  Synthetic sequence
cagccggtcagaccagtagc

SEQ ID NO: 174 - DNA, 20 bp, Artificial Sequence
  Synthetic sequence
gctagtaacgtactgtttgc

SEQ ID NO: 175 - DNA, 20 bp, Artificial Sequence
  Synthetic sequence
aagaaggctagtaacgtact

SEQ ID NO: 176 - DNA, 20 bp, Artificial Sequence
  Synthetic sequence
gaagaaggctagtaacgtac

SEQ ID NO: 177 - DNA, 20 bp, Artificial Sequence
  Synthetic sequence
agaagaaggctagtaacgta

SEQ ID NO: 178 - DNA, 20 bp, Artificial Sequence
  Synthetic sequence
gagaaaggttccgtcgggct

SEQ ID NO: 179 - DNA, 20 bp, Artificial Sequence
  Synthetic sequence
ggagaaaggttccgtcgggc

SEQ ID NO: 180 - DNA, 20 bp, Artificial Sequence
  Synthetic sequence
cggattcggtcccccagtga

SEQ ID NO: 181 - DNA, 20 bp, Artificial Sequence
  Synthetic sequence
ccggattcggtcccccagtg

SEQ ID NO: 182 - DNA, 20 bp, Artificial Sequence
  Synthetic sequence
atcccggattcggtccccca

SEQ ID NO: 183 - DNA, 20 bp, Artificial Sequence
  Synthetic sequence
agatcccggattcggtcccc

SEQ ID NO: 184 - DNA, 20 bp, Artificial Sequence
  Synthetic sequence
aagatcccggattcggtccc

SEQ ID NO: 185 - DNA, 20 bp, Artificial Sequence
  Synthetic sequence
gcaagatcccggattcggtc

SEQ ID NO: 186 - DNA, 20 bp, Artificial Sequence
  Synthetic sequence
tgagcaagatcccggattcg

SEQ ID NO: 187 - DNA, 20 bp, Artificial Sequence
  Synthetic sequence
tgatacggggggatggagtg

SEQ ID NO: 188 - DNA, 20 bp, Artificial Sequence
  Synthetic sequence
ttgatacggggggatggagt

SEQ ID NO: 189 - DNA, 20 bp, Artificial Sequence
  Synthetic sequence
gggagaggccttgatacggg

SEQ ID NO: 190 - DNA, 20 bp, Artificial Sequence
  Synthetic sequence
gatcaccaactggggttggc

SEQ ID NO: 191 - DNA, 20 bp, Artificial Sequence
  Synthetic sequence
tgcgtgtgcgcgctgcaggt

SEQ ID NO: 192 - DNA, 20 bp, Artificial Sequence
  Synthetic sequence
gtgcgtgtgcgcgctgcagg

SEQ ID NO: 193 - DNA, 20 bp, Artificial Sequence
  Synthetic sequence
gcccttgtacttttgcatag